(19) United States
(12) Patent Application Publication
(10) Pub. No.: US 2009/0054423 A1
(43) Pub. Date: Feb. 26, 2009

(54) Niacin receptor agonists, compositions containing such compounds and methods of treatment

(76) Inventors: Jason E. Imbriglio, Piscataway, NJ (US); Steven L. Colletti, Princeton Junction, NJ (US); James R. Tata, Westfield, NJ (US); Rui Liang, East Brunswick, NJ (US); Subharekha Raghavan, Teaneck, NJ (US); Darby R. Schmidt, Clark, NJ (US); Abigail L. Smenton, Brooklyn, NY (US); Sook Yee Chan, Fords, NJ (US)

Correspondence Address: MERCK AND CO., INC, P O BOX 2000, RAHWAY, NJ, 07065-0907, US

(21) Appl. No.: 11/918,270

(22) PCT Filed: Apr. 7, 2006
(86) PCT No.: PCT/US2006/012876

§ 371 Date: Oct. 11, 2007

Related U.S. Application Data
(60) Provisional application No. 60/670,764, filed on Apr. 13, 2005.

(51) Int. Cl. A61K 31/538; C07D 257/04; A61K 31/41; C07D 231/54; A61K 31/416; C07D 261/06; A61K 31/42; C07D 403/02; A61P 9/10; A61P 3/10; A61K 31/506; C07D 401/02; A61K 31/4439; C07D 277/20; A61K 31/426; A61K 31/497; A61K 31/437

(52) U.S. Cl. 514/230.5; 548/250; 514/381; 548/360.1; 514/406; 548/247; 514/378; 544/333; 514/256; 546/275.7; 514/339; 548/204; 514/365; 544/405; 514/255.05; 514/292; 514/294

(57) ABSTRACT

The present invention encompasses compounds of Formula I: as well as pharmaceutically acceptable salts and hydrates thereof, that are useful for treating dyslipidemias. Pharmaceutical compositions and methods of use are also included.

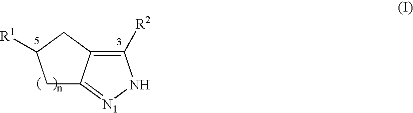

### BACKGROUND OF THE INVENTION

The present invention relates to compounds, compositions and methods of treatment or prevention in a mammal relating to dyslipidemias. Dyslipidemia is a condition wherein serum lipids are abnormal. Elevated cholesterol and low levels of high density lipoprotein (HDL) are associated with a greater-than-normal risk of atherosclerosis and cardiovascular disease. Factors known to affect serum cholesterol include genetic predisposition, diet, body weight, degree of physical activity, age and gender. While cholesterol in normal amounts is a vital building block for essential organic molecules such as steroids, cell membranes, and bile acids, cholesterol in excess is known to contribute to cardiovascular disease. For example, cholesterol is a primary component of plaque which collects in coronary arteries, resulting in the cardiovascular disease termed atherosclerosis.

Traditional therapies for reducing cholesterol include medications such as statins (which reduce production of cholesterol by the body). More recently, the value of nutrition and nutritional supplements in reducing blood cholesterol has received significant attention. For example, dietary compounds such as soluble fiber, vitamin E, soy, garlic, omega-3 fatty acids, and niacin have all received significant attention and research funding.

Niacin or nicotinic acid (pyridine-3-carboxylic acid) is a drug that reduces coronary events in clinical trials. It is commonly known for its effect in elevating serum levels of high density lipoproteins (HDL). Importantly, niacin also has a beneficial effect on other lipid profiles. Specifically, it reduces low density lipoproteins (LDL), very low density lipoproteins (VLDL), and triglycerides (TG). However, the clinical use of nicotinic acid is limited by a number of adverse side-effects including cutaneous vasodilation, sometimes called flushing.

Despite the attention focused on traditional and alternative means for controlling serum cholesterol, serum triglycerides, and the like, a significant portion of the population has total cholesterol levels greater than about 200 mg/dL, and are thus candidates for dyslipidemia therapy. There thus remains a need in the art for compounds, compositions and alternative methods of reducing total cholesterol, serum triglycerides, and the like, and raising HDL.

The present invention relates to compounds that have been discovered to have effects in modifying serum lipid levels.

The invention thus provides compositions for effecting reduction in total cholesterol and triglyceride concentrations and raising HDL, in accordance with the methods described.

Consequently one object of the present invention is to provide a nicotinic acid receptor agonist that can be used to treat dyslipidemias, atherosclerosis, diabetes, metabolic syndrome and related conditions while minimizing the adverse effects that are associated with niacin treatment.

Yet another object is to provide a pharmaceutical composition for oral use.

These and other objects will be apparent from the description provided herein.

### SUMMARY OF THE INVENTION

The present invention relates to a compound of Formula I:

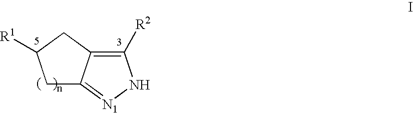

or a pharmaceutically acceptable salt or solvate thereof, wherein:

n represents 1 or 2;

R¹ is selected from the group consisting of cyclohexyl, phenyl and heteroaryl containing 5-6 atoms, said heteroaryl 5-membered rings containing 1-4 heteroatoms, 0-1 of which are O or S and 0-4 of which are N, and said Heteroaryl 6-membered rings containing 1-3 N atoms,

said cyclohexyl, phenyl and heteroaryl being optionally substituted with 1-4 members selected from the group consisting of: halogen, OH, SH, CN, nitro, C₁₋₄ haloalkyl, amino, C₁₋₄ alkylamino, C₂₋₈ dialkylamino, C₁₋₄ alkyl, C₁₋₄ alkoxy, C₂₋₄alkenyl, C₂₋₄alkynyl, C₃₋₆ cycloalkyl, C₁₋₄haloalkoxy, C₁₋₄alkylthio, C₁₋₄alkylsulfinyl and C₁₋₄alkylsulfonyl, and

R² is

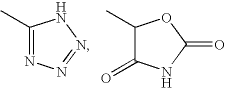

or CO₂R^{a} wherein R^{a} is H or C₁₋₄alkyl.

### DETAILED DESCRIPTION OF THE INVENTION

The invention is described herein in detail using the terms defined below unless otherwise specified.

“Alkyl”, as well as other groups having the prefix “alk”, such as alkoxy, alkanoyl and the like, means carbon chains which may be linear, branched, or cyclic, or combinations thereof, containing the indicated number of carbon atoms. If no number is specified, 1-6 carbon atoms are intended for linear and 3-7 carbon atoms for branched alkyl groups. Examples of alkyl groups include methyl, ethyl, propyl, isopropyl, butyl, sec- and tert-butyl, pentyl, hexyl, heptyl, octyl, nonyl and the like. Cycloalkyl is a subset of alkyl; if no number of atoms is specified, 3-7 carbon atoms are intended, forming 1-3 carbocyclic rings that are fused. “Cycloalkyl” also includes monocyclic rings fused to an aryl group in which the point of attachment is on the non-aromatic portion. Examples of cycloalkyl include cyclopropyl, cyclobutyl, cyclopentyl, cyclohexyl, cycloheptyl, tetrahydronaphthyl, decahydronaphthyl, indanyl and the like.

“Alkenyl” means carbon chains which contain at least one carbon-carbon double bond, and which may be linear or branched or combinations thereof. Examples of alkenyl include vinyl, allyl, isopropenyl, pentenyl, hexenyl, heptenyl, 1-propenyl, 2-butenyl, 2-methyl-2-butenyl, and the like.

“Alkynyl” means carbon chains which contain at least one carbon-carbon triple bond, and which may be linear or branched or combinations thereof. Examples of alkynyl include ethynyl, propargyl, 3-methyl-1-pentynyl, 2-heptynyl and the like.

“Heteroaryl” (HAR) unless otherwise specified, means a mono-aromatic ring or ring system containing at least one heteroatom selected from O, S and N, with 5 to 6 atoms. Examples include, but are not limited to, pyrrolyl, isoxazolyl, isothiazolyl, pyrazolyl, pyridyl, oxazolyl, oxadiazolyl, thiadiazolyl, thiazolyl, imidazolyl, triazolyl, tetrazolyl, furanyl, triazinyl, thienyl, pyrimidyl, pyridazinyl, pyrazinyl and the like. Heteroaryl also includes such groups in charged form, e.g., pyridinium.

“Halogen” (Halo) includes fluorine, chlorine, bromine and iodine.

The phrase “in the absence of substantial flushing” refers to the elimination of measurable cutaneous flushing, e.g., a side effect that is often seen when nicotinic acid is administered in therapeutic amounts. The flushing effect of nicotinic acid usually becomes less frequent and less severe as the patient develops tolerance to the drug at therapeutic doses, but the flushing effect still occurs to some extent and can be transient. Thus, “in the absence of substantial flushing” refers to the reduced severity of flushing when it occurs, or fewer flushing events than would otherwise occur. Preferably, the incidence of flushing (relative to niacin) is reduced by at least about a third, more preferably the incidence is reduced by half, and most preferably, the flushing incidence is reduced by about two thirds or more. Likewise, the severity of flushing (relative to niacin) is preferably reduced by at least about a third, more preferably by at least half, and most preferably by at least about two thirds. Clearly a one hundred percent reduction in flushing incidence and severity is most preferable, but is not required.

One aspect of the invention relates to a compound of Formula I:

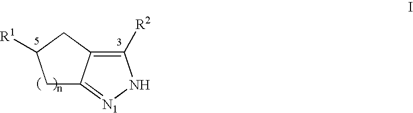

or a pharmaceutically acceptable salt or solvate thereof, wherein:

n represents 1 or 2;

R¹ is selected from the group consisting of cyclohexyl, phenyl and heteroaryl containing 5-6 atoms, said heteroaryl 5-membered rings containing 1-4 heteroatoms, 0-1 of which are O or S and 0-4 of which are N, and said Heteroaryl 6-membered rings containing 1-3 N atoms,

said cyclohexyl, phenyl and heteroaryl being optionally substituted with 14 members selected from the group consisting of: halogen, OH, SH, CN, nitro, C₁₋₄ haloalkyl, amino, C₁₋₄ alkylamino, C₂₋₈ dialkylamino, C₁₋₄ alkyl, C₁₋₄ alkoxy, C₂₋₄alkenyl, C₂₋₄alkynyl, C₃₋₆ cycloalkyl, C₁₋₄haloalkoxy, C₁₋₄alkylthio C₁₋₄alkylsulfinyl and C₁₋₄alkylsulfonyl, and

R² is

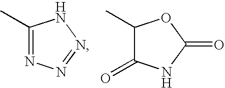

or CO₂R^{a} wherein R^{a} is H or C₁₋₄alkyl.

One subset of compounds that is of interest relates to a compound of formula I or a pharmaceutically acceptable salt or solvate thereof wherein n is 1. Within this subset, all other variables are as originally defined.

Another subset of compounds that is of interest relates to a compound of formula I or a pharmaceutically acceptable salt or solvate thereof wherein n is 2. Within this subset, all other variables are as originally defined.

Another subset of compounds that is of interest relates to a compound of formula I or a pharmaceutically acceptable salt or solvate there of wherein R¹ represents phenyl or heteroaryl, said group being optionally substituted with 1-4 groups, 1-4 of which are halo groups and 1-2 of which are selected from the group consisting of: OH, SH, CN, nitro, C₁₋₄ haloalkyl, amino, C₁₋₄ alkylamino, C₂₋₈ dialkylamino, C₁₋₄ alkyl, C₁₋₄ alkoxy, C₂₋₄alkenyl, C₂₋₄alkynyl, C₃₋₆ cycloalkyl, C₁₋₄haloalkoxy, C₁₋₄alkylthio, C₁₋₄alkylsulfinyl, and C₁₋₄alkylsulfonyl. Within this subset, all other variables are as originally defined.

Another subset of compounds that is of particular interest relates to a compound of formula I or a pharmaceutically acceptable salt or solvate thereof wherein R¹ represents phenyl optionally substituted with 1-4 groups, 1-4 of which are halo groups and 1-2 of which are selected from the group consisting of: OH, SH, CN, nitro, C₁₋₄ haloalkyl, amino, C₁₋₄ alkylamino, C₂₋₈ dialkylamino, C₁₋₄ alkyl, C₁₋₄ alkoxy, C₂₋₄alkenyl, C₂₋₄alkynyl, C₃₋₆ cycloalkyl, C₁₋₄haloalkoxy, C₁₋₄alkylthio, C₁₋₄alkylsulfinyl, and C₁₋₄alkylsulfonyl. Within this subset, all other variables are as originally defined.

Another subset of compounds that is of particular interest relates to a compound of formula I or a pharmaceutically acceptable salt or solvate thereof wherein R¹ represents heteroaryl optionally substituted with 1-4 groups, 1-4 of which are halo groups and 1-2 of which are selected from the group consisting of: OH, SH, CN, nitro, C₁₋₄ haloalkyl, amino, C₁₋₄ alkylamino, C₂₋₈ dialkylamino, C₁₋₄ alkyl, C₁₋₄ alkoxy, C₂₋₄alkenyl, C₂₋₄alkynyl, C₃₋₆ cycloalkyl, C₁₋₄haloalkoxy, C₁₋₄alkylthio, C₁₋₄alkylsulfinyl, and C₁₋₄alkylsulfonyl. Within this subset, all other variables are as originally defined.

Another subset of compounds that is of particular interest relates to a compound of formula I or a pharmaceutically acceptable salt or solvate thereof wherein R¹ represents heteroaryl optionally substituted with 1-4 groups, 1-4 of which are halo groups and 1-2 of which are C₁₋₄ haloalkyl or C₁₋₄ alkyl. Within this subset, all other variables are as originally defined.

Another subset of compounds that is of particular interest relates to a compound of formula I or a pharmaceutically acceptable salt or solvate thereof wherein R¹ represents phenyl optionally substituted with 1-4 halo groups. Within this subset, all other variables are as originally defined.

Another subset of compounds that is of interest relates to compounds of formula I or a pharmaceutically acceptable salt or solvate thereof wherein R² represents CO₂R^{a} and R^{a} represents H. Within this subset, all other variables are as originally defined.

Another subset of compounds that is of interest relates to compounds of formula I or a pharmaceutically acceptable salt or solvate thereof wherein R² represents tetrazolyl. Within this subset, all other variables are as originally defined.

Examples of compounds that are of particular interest include those in the following tables:

TABLE A

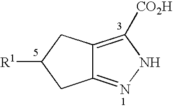

R¹ =

| | | |
|---|---|---|
| 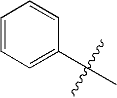 | 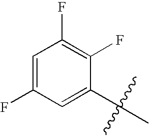 | 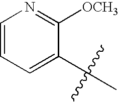 |
| 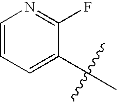 | 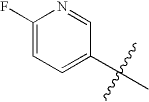 | 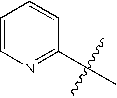 |
| 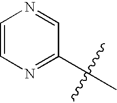 | 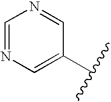 | 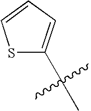 |
| 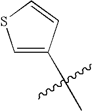 | 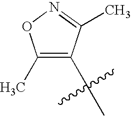 | 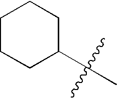 |
| 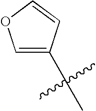 | 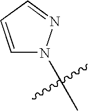 | 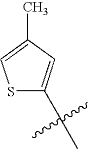 |
| 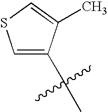 | 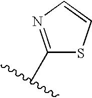 | 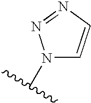 |
| 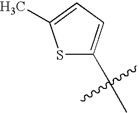 | 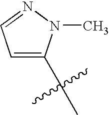 | 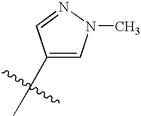 |
| 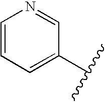 | 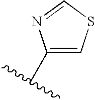 | 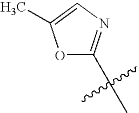 |

as well as the pharmaceutically acceptable salts and solvates thereof.

TABLE B

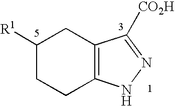

R¹ =

| | | |
|---|---|---|
| 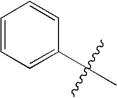 | 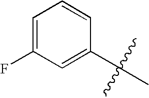 | 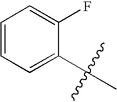 |
| 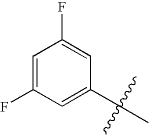 | 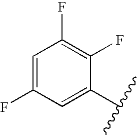 | |
| 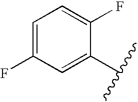 | 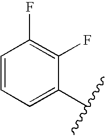 | 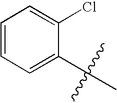 |
| 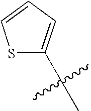 | 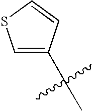 | 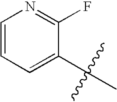 |
| 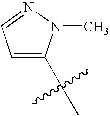 | 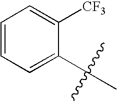 | 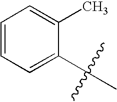 |

as well as the pharmaceutically acceptable salts and solvates thereof.

TABLE C

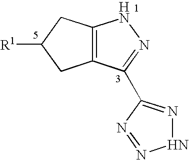

R¹ =

| | |
|---|---|
| 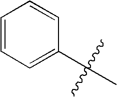 | 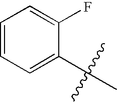 |
| 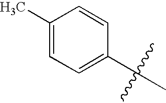 | 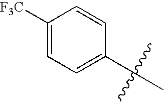 |
| 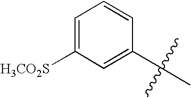 | 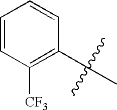 |
| 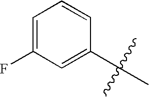 | 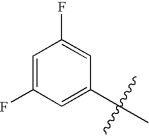 |
| 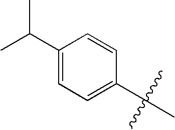 | 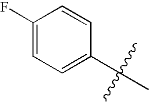 |
| 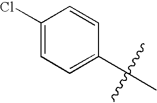 | 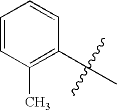 |
| 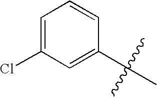 | 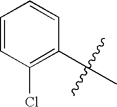 |
| 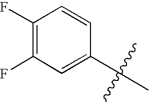 | 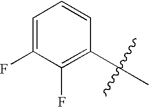 |
| 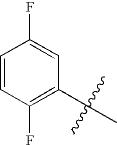 | 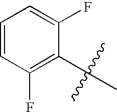 |
| 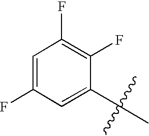 | 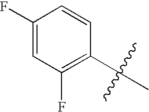 |
| 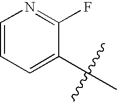 | 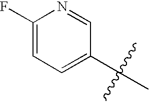 |
| 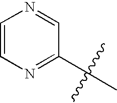 | 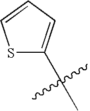 |
| 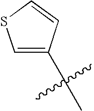 | 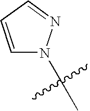 |
| 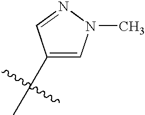 | |

as well as the pharmaceutically acceptable salts and solvates thereof.

TABLE D

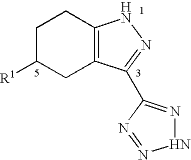

R¹ =

| | | |
|---|---|---|
| 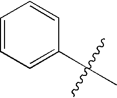 | 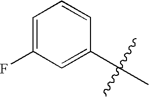 | 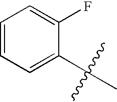 |
| 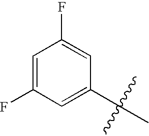 | 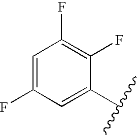 | |
| 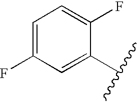 | 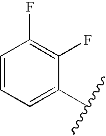 | 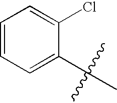 |
| 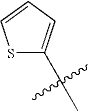 | 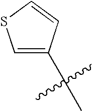 | 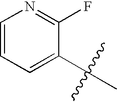 |
| 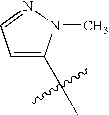 | 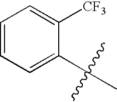 | 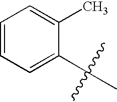 |

as well as the pharmaceutically acceptable salts and solvates thereof.

### Dosing Information

The dosages of compounds of formula I or a pharmaceutically acceptable salt or solvate thereof vary within wide limits. The specific dosage regimen and levels for any particular patient will depend upon a variety of factors including the age, body weight, general health, sex, diet, time of administration, route of administration, rate of excretion, drug combination and the severity of the patient's condition. Consideration of these factors is well within the purview of the ordinarily skilled clinician for the purpose of determining the therapeutically effective or prophylactically effective dosage amount needed to prevent, counter, or arrest the progress of the condition. Generally, the compounds will be administered in amounts ranging from as low as about 0.01 mg/day to as high as about 2000 mg/day, in single or divided doses. A representative dosage is about 0.1 mg/day to about 1 g/day. Lower dosages can be used initially, and dosages increased to further minimize any untoward effects. It is expected that the compounds described herein will be administered on a daily basis for a length of time appropriate to treat or prevent the medical condition relevant to the patient, including a course of therapy lasting months, years or the life of the patient.

### Combination Therapy

One or more additional active agents may be administered with the compounds described herein. The additional active agent or agents can be lipid modifying compounds or agents having other pharmaceutical activities, or agents that have both lipid-modifying effects and other pharmaceutical activities. Examples of additional active agents which may be employed include but are not limited to HMG-CoA reductase inhibitors, which include statins in their lactonized or dihydroxy open acid forms and pharmaceutically acceptable salts and esters thereof, including but not limited to lovastatin (see U.S. Pat. No. 4,342,767), simvastatin (see U.S. Pat. No. 4,444,784), dihydroxy open-acid simvastatin, particularly the ammonium or calcium salts thereof, pravastatin, particularly the sodium salt thereof (see U.S. Pat. No. 4,346,227), fluvastatin particularly the sodium salt thereof (see U.S. Pat. No. 5,354,772), atorvastatin, particularly the calcium salt thereof (see U.S. Pat. No. 5,273,995), pitavastatin also referred to as NK-104 (see PCT international publication number WO 97/23200) and rosuvastatin, also known as CRESTOR®; see U.S. Pat. No. 5,260,440); HMG-CoA synthase inhibitors; squalene epoxidase inhibitors; squalene synthetase inhibitors (also known as squalene synthase inhibitors), acyl-coenzyme A: cholesterol acyltransferase (ACAT) inhibitors including selective inhibitors of ACAT-1 or ACAT-2 as well as dual inhibitors of ACAT-1 and -2; microsomal triglyceride transfer protein (MTP) inhibitors; endothelial lipase inhibitors; bile acid sequestrants; LDL receptor inducers; platelet aggregation inhibitors, for example glycoprotein IIb/IIIa fibrinogen receptor antagonists and aspirin; human peroxisome proliferator activated receptor gamma (PPAR-gamma) agonists including the compounds commonly referred to as glitazones for example pioglitazone and rosiglitazone and, including those compounds included within the structural class known as thiazolidine diones as well as those PPAR-gamma agonists outside the thiazolidine dione structural class; PPAR-alpha agonists such as clofibrate, fenofibrate including micronized fenofibrate, and gemfibrozil; PPAR dual alpha/gamma agonists; vitamin B6 (also known as pyridoxine) and the pharmaceutically acceptable salts thereof such as the HCl salt; vitamin B12 (also known as cyanocobalamin); folic acid or a pharmaceutically acceptable salt or ester thereof such as the sodium salt and the methylglucamine salt; anti-oxidant vitamins such as vitamin C and E and beta carotene; beta-blockers; angiotensin II antagonists such as losartan; angiotensin converting enzyme inhibitors such as enalapril and captopril; renin inhibitors, calcium channel blockers such as nifedipine and diltiazem; endothelin antagonists; agents that enhance ABCA1 gene expression; cholesteryl ester transfer protein (CETP) inhibiting compounds, 5-lipoxygenase activating protein (FLAP) inhibiting compounds, 5-lipoxygenase (5-LO) inhibiting compounds, farnesoid X receptor (FXR) ligands including both antagonists and agonists; Liver X Receptor (LXR)-alpha ligands, LXR-beta ligands, bisphosphonate compounds such as alendronate sodium; cyclooxygenase-2 inhibitors such as rofecoxib and celecoxib; and compounds that attenuate vascular inflammation.

Cholesterol absorption inhibitors can also be used in the present invention. Such compounds block the movement of cholesterol from the intestinal lumen into enterocytes of the small intestinal wall, thus reducing serum cholesterol levels. Examples of cholesterol absorption inhibitors are described in U.S. Pat. Nos. 5,846,966, 5,631,365, 5,767,115, 6,133,001, 5,886,171, 5,856,473, 5,756,470, 5,739,321, 5,919,672, and in PCT application Nos. WO 00/63703, WO 00/60107, WO 00/38725, WO 00/34240, WO 00/20623, WO 97/45406, WO 97/16424, WO 97/16455, and WO 95/08532. The most notable cholesterol absorption inhibitor is ezetimibe, also known as 1-(4-fluorophenyl)-3(R)-[3(S)-(4-fluorophenyl)-3-hydroxypropyl)]-4(S)-(4-hydroxyphenyl)-2-azetidinone, described in U.S. Pat. Nos. 5,767,115 and 5,846,966.

Therapeutically effective amounts of cholesterol absorption inhibitors include dosages of from about 0.01 mg/kg to about 30 mg/kg of body weight per day, preferably about 0.1 mg/kg to about 15 mg/kg.

For diabetic patients, the compounds used in the present invention can be administered with conventional diabetic medications. For example, a diabetic patient receiving treatment as described herein may also be taking insulin or an oral antidiabetic medication. One example of an oral antidiabetic medication useful herein is metformin.

In the event that these niacin receptor agonists induce some degree of vasodilation, it is understood that the compounds of formula I can be co-dosed with a vasodilation suppressing agent. Consequently, one aspect of the methods described herein relates to the use of a compound of formula I or a pharmaceutically acceptable salt or solvate thereof in combination with a compound that reduces flushing. Conventional compounds such as aspirin, ibuprofen, naproxen, indomethacin, other NSAIDs, COX-2 selective inhibitors and the like are useful in this regard, at conventional doses. Alternatively, DP antagonists are useful as well.

Different subtypes of receptors interact with prostaglandin D2. One prostaglandin D2 receptor is referred to as “DP” and another prostaglandin D2 receptor is known as “CRTH2”. The present invention utilizes antagonism of the DP receptor to prevent, minimize or reduce flushing that otherwise may occur.

Doses of the DP receptor antagonist and selectivity are such that the DP antagonist selectively modulates the DP receptor without substantially modulating the CRTH2 receptor. In particular, the DP receptor antagonist ideally has an affinity at the DP receptor (i.e., Kᵢ) that is at least about 10 times higher (a numerically lower K; value) than the affinity at the CRTH2 receptor. Any compound that selectively interacts with DP according to these guidelines is deemed “DP selective”.

Dosages for DP antagonists as described herein, that are useful for reducing or preventing the flushing effect in mammalian patients, particularly humans, include dosages ranging from as low as about 0.01 mg/day to as high as about 100 mg/day, administered in single or divided daily doses. Preferably the dosages are from about 0.1 mg/day to as high as about 1.0 g/day, in single or divided daily doses.

Examples of compounds that are particularly useful for selectively antagonizing DP receptors and suppressing the flushing effect include the following:

| | |
|---|---|
| | Compound A |
| 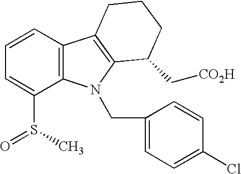 | |
| | Compound B |
| 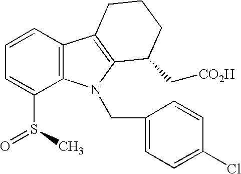 | |
| | Compound C |
| 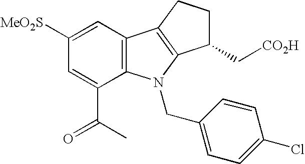 | |
| | Compound D |
| 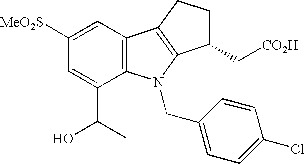 | |
| | Compound E |
| 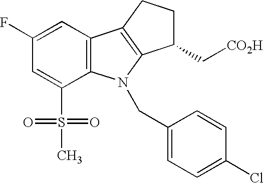 | |
| | Compound F |
| 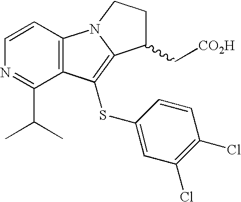 | |
| | Compound G |
| 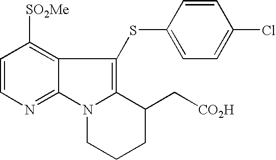 | |
| | Compound H |
| 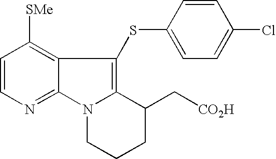 | |
| | Compound I |
| 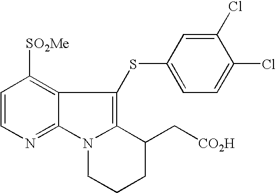 | |
| | Compound J |
| 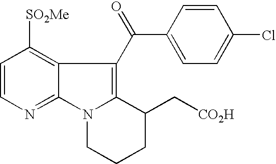 | |
| | Compound K |
| 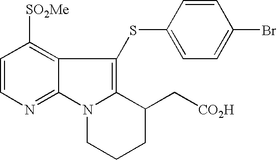 | |
| | Compound L |
| 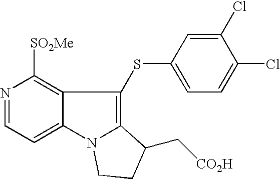 | |
| | Compound M |
| 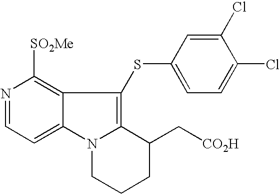 | |
| | Compound N |
| 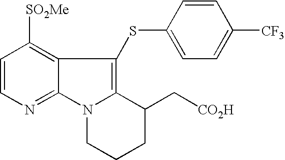 | |
| | Compound O |
| 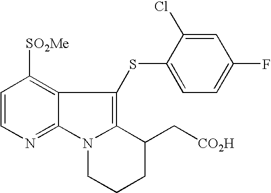 | |
| | Compound P |
| 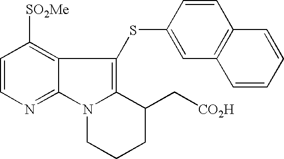 | |
| | Compound Q |
| 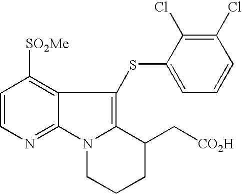 | |
| | Compound R |
| 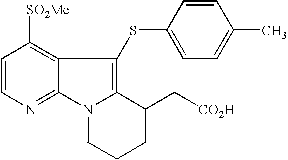 | |
| | Compound S |
| 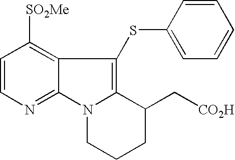 | |
| | Compound T |
| 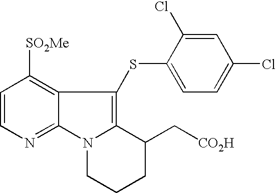 | |
| | Compound U |
| 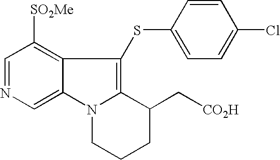 | |
| | Compound V |
| 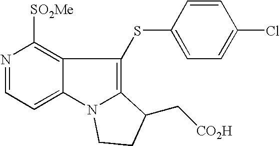 | |
| | Compound W |
| 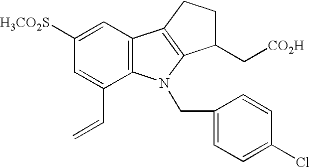 | |
| | Compound X |
| 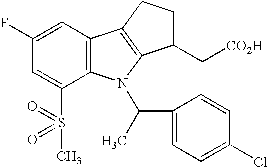 | |
| | Compound Y |
| 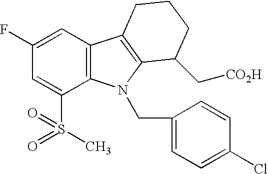 | |
| | Compound Z |
| 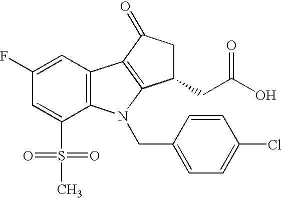 | |
| | Compound AA |
| 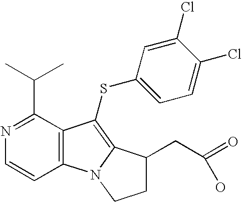 | |
| | Compound AB |
| 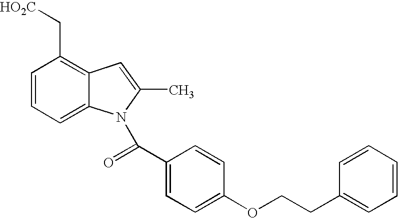 | |
| | Compound AC |
| 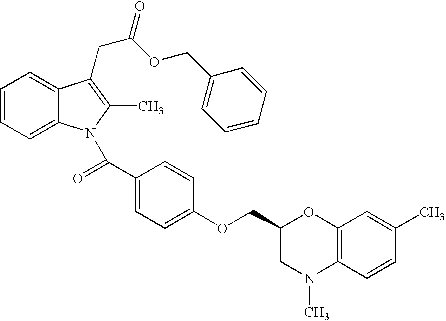 | |
| | Compound AD |
| 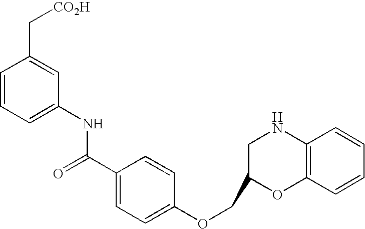 | |
| | Compound AE |
| 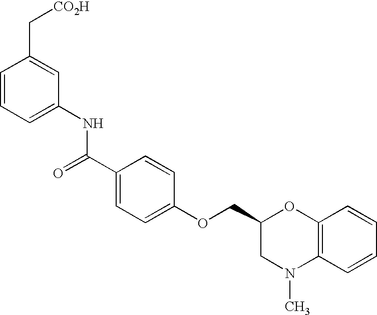 | |
| | Compound AF |
| 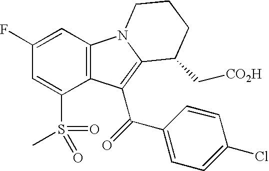 | |
| | Compound AG |
| 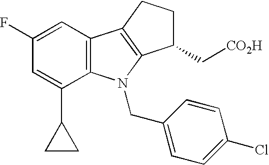 | |
| | Compound AH |
| 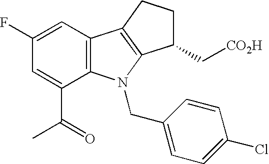 | |
| | Compound AI |
| 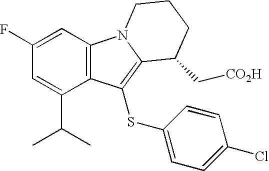 | |
| | Compound AJ |
| 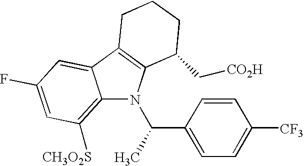 | |

as well as the pharmaceutically acceptable salts and solvates thereof.

The compound of formula I or a pharmaceutically acceptable salt or solvate thereof and the DP antagonist can be administered together or sequentially in single or multiple daily doses, e.g., bid, tid or qid, without departing from the invention. If sustained release is desired, such as a sustained release product showing a release profile that extends beyond 24 hours, dosages may be administered every other day. However, single daily doses are preferred. Likewise, morning or evening dosages can be utilized.

### Salts and Solvates

Salts and solvates of the compounds of formula I are also included in the present invention, and numerous pharmaceutically acceptable salts and solvates of nicotinic acid are useful in this regard. Alkali metal salts, in particular, sodium and potassium, form salts that are useful as described herein. Likewise alkaline earth metals, in particular, calcium and magnesium, form salts that are useful as described herein. Various salts of amines, such as ammonium and substituted ammonium compounds also form salts that are useful as described herein. Similarly, solvated forms of the compounds of formula I, including hydrates, such as the hemihydrate, mono-, di-, tri- and sesquihydrate are of particular interest.

The compounds used in the present invention can be administered via any conventional route of administration. The preferred route of administration is oral.

### Pharmaceutical Compositions

The pharmaceutical compositions described herein are generally comprised of a compound of formula I or a pharmaceutically acceptable salt or solvate thereof, in combination with a pharmaceutically acceptable carrier.

Examples of suitable oral compositions include tablets, capsules, troches, lozenges, suspensions, dispersible powders or granules, emulsions, syrups and elixirs. Examples of carrier ingredients include diluents, binders, disintegrants, lubricants, sweeteners, flavors, colorants, preservatives, and the like. Examples of diluents include, for example, calcium carbonate, sodium carbonate, lactose, calcium phosphate and sodium phosphate. Examples of granulating and disintegrants include corn starch and alginic acid. Examples of binding agents include starch, gelatin and acacia. Examples of lubricants include magnesium stearate, calcium stearate, stearic acid and talc. The tablets may be uncoated or coated by known techniques. Such coatings may delay disintegration and thus, absorption in the gastrointestinal tract and thereby provide a sustained action over a longer period.

In one embodiment of the invention, a compound of formula I or a pharmaceutically acceptable salt or solvate thereof is combined with another therapeutic agent and the carrier to form a fixed combination product. This fixed combination product may be a tablet or capsule for oral use.

More particularly, in another embodiment of the invention, a compound of formula I or a pharmaceutically acceptable salt or solvate thereof (about 1 to about 1000 mg) and the second therapeutic agent (about 1 to about 500 mg) are combined with the pharmaceutically acceptable carrier, providing a tablet or capsule for oral use.

Sustained release over a longer period of time may be particularly important in the formulation. A time delay material such as glyceryl monostearate or glyceryl distearate may be employed. The dosage form may also be coated by the techniques described in the U.S. Pat. Nos. 4,256,108; 4,166,452 and 4,265,874 to form osmotic therapeutic tablets for controlled release.

Other controlled release technologies are also available and are included herein. Typical ingredients that are useful to slow the release of nicotinic acid in sustained release tablets include various cellulosic compounds, such as methylcellulose, ethylcellulose, propylcellulose, hydroxypropylcellulose, hydroxyethylcellulose, hydroxypropylmethylcellulose, microcrystalline cellulose, starch and the like. Various natural and synthetic materials are also of use in sustained release formulations. Examples include alginic acid and various alginates, polyvinyl pyrrolidone, tragacanth, locust bean gum, guar gum, gelatin, various long chain alcohols, such as cetyl alcohol and beeswax.

Optionally and of even more interest is a tablet as described above, comprised of a compound of formula I or a pharmaceutically acceptable salt or solvate thereof, and further containing an HMG Co-A reductase inhibitor, such as simvastatin or atorvastatin. This particular embodiment optionally contains the DP antagonist as well.

Typical release time frames for sustained release tablets in accordance with the present invention range from about 1 to as long as about 48 hours, preferably about 4 to about 24 hours, and more preferably about 8 to about 16 hours.

Hard gelatin capsules constitute another solid dosage form for oral use. Such capsules similarly include the active ingredients mixed with carrier materials as described above. Soft gelatin capsules include the active ingredients mixed with water-miscible solvents such as propylene glycol, PEG and ethanol, or an oil such as peanut oil, liquid paraffin or olive oil.

Aqueous suspensions are also contemplated as containing the active material in admixture with excipients suitable for the manufacture of aqueous suspensions. Such excipients include suspending agents, for example sodium carboxymethylcellulose, methylcellulose, hydroxypropylmethylcellulose, sodium alginate, polyvinylpyrrolidone, tragacanth and acacia; dispersing or wetting agents, e.g., lecithin; preservatives, e.g., ethyl, or n-propyl para-hydroxybenzoate, colorants, flavors, sweeteners and the like.

Dispersible powders and granules suitable for preparation of an aqueous suspension by the addition of water provide the active ingredients in admixture with a dispersing or wetting agent, suspending agent and one or more preservatives. Suitable dispersing or wetting agents and suspending agents are exemplified by those already mentioned above.

Syrups and elixirs may also be formulated.

More particularly, a pharmaceutical composition that is of interest is a sustained release tablet that is comprised of a compound of formula I or a pharmaceutically acceptable salt or solvate thereof, and a DP receptor antagonist that is selected from the group consisting of compounds A through AJ in combination with a pharmaceutically acceptable carrier.

Yet another pharmaceutical composition that is of more interest is comprised of a compound of formula I or a pharmaceutically acceptable salt or solvate thereof and a DP antagonist compound selected from the group consisting of compounds A, B, D, E, X, AA, AF, AG, AH, AI and AJ, in combination with a pharmaceutically acceptable carrier.

Yet another pharmaceutical composition that is of more particular interest relates to a sustained release tablet that is comprised of a compound of formula I or a pharmaceutically acceptable salt or solvate thereof, a DP receptor antagonist selected from the group consisting of compounds A, B, D, E, X, AA, AF, AG, AH, AI and AJ, and simvastatin or atorvastatin in combination with a pharmaceutically acceptable carrier.

The term “composition”, in addition to encompassing the pharmaceutical compositions described above, also encompasses any product which results, directly or indirectly, from the combination, complexation or aggregation of any two or more of the ingredients, active or excipient, or from dissociation of one or more of the ingredients, or from other types of reactions or interactions of one or more of the ingredients. Accordingly, the pharmaceutical composition of the present invention encompasses any composition made by admixing or otherwise combining the compounds, any additional active ingredient(s), and the pharmaceutically acceptable excipients.

Another aspect of the invention relates to the use of a compound of formula I or a pharmaceutically acceptable salt or solvate thereof and a DP antagonist in the manufacture of a medicament. This medicament has the uses described herein.

More particularly, another aspect of the invention relates to the use of a compound of formula I or a pharmaceutically acceptable salt or solvate thereof, a DP antagonist and an HMG Co-A reductase inhibitor, such as simvastatin, in the manufacture of a medicament. This medicament has the uses described herein.

Compounds of the present invention demonstrate anti-hyperlipidemic activity, indicative of utility for reducing LDL-C, triglycerides, apolipoprotein a and total cholesterol, and increasing HDL-C. Consequently, the compounds of the present invention are useful in treating dyslipidemias. The present invention thus relates to the treatment, prevention or reversal of atherosclerosis and the other diseases and conditions described herein, by administering a compound of formula I or a pharmaceutically acceptable salt or solvate in an amount that is effective for treating, preventing or reversing said condition. This is achieved in humans by administering a compound of formula I or a pharmaceutically acceptable salt or solvate thereof in an amount that is effective to treat or prevent said condition, while preventing, reducing or minimizing flushing effects in terms of frequency and/or severity.

One aspect of the invention that is of interest is a method of treating atherosclerosis in a human patient in need of such treatment comprising administering to the patient a compound of formula I or a pharmaceutically acceptable salt or solvate thereof in an amount that is effective for treating atherosclerosis in the absence of substantial flushing.

Another aspect of the invention that is of interest relates to a method of raising serum HDL levels in a human patient in need of such treatment, comprising administering to the patient a compound of formula I or a pharmaceutically acceptable salt or solvate thereof in an amount that is effective for raising serum HDL levels.

Another aspect of the invention that is of interest relates to a method of treating dyslipidemia in a human patient in need of such treatment comprising administering to the patient a compound of formula I or a pharmaceutically acceptable salt or solvate thereof in an amount that is effective for treating dyslipidemia.

Another aspect of the invention that is of interest relates to a method of reducing serum VLDL or LDL levels in a human patient in need of such treatment, comprising administering to the patient a compound of formula I or a pharmaceutically acceptable salt or solvate thereof in an amount that is effective for reducing serum VLDL or LDL levels in the patient in the absence of substantial flushing.

Another aspect of the invention that is of interest relates to a method of reducing serum triglyceride levels in a human patient in need of such treatment, comprising administering to the patient a compound of formula I or a pharmaceutically acceptable salt or solvate thereof in an amount that is effective for reducing serum triglyceride levels.

Another aspect of the invention that is of interest relates to a method of reducing serum Lp(a) levels in a human patient in need of such treatment, comprising administering to the patient a compound of formula I or a pharmaceutically acceptable salt or solvate thereof in an amount that is effective for reducing serum Lp(a) levels. As used herein Lp(a) refers to lipoprotein (a).

Another aspect of the invention that is of interest relates to a method of treating diabetes, and in particular, type 2 diabetes, in a human patient in need of such treatment comprising administering to the patient a compound of formula I or a pharmaceutically acceptable salt or solvate thereof in an amount that is effective for treating diabetes.

Another aspect of the invention that is of interest relates to a method of treating metabolic syndrome in a human patient in need of such treatment comprising administering to the patient a compound of formula I or a pharmaceutically acceptable salt or solvate thereof in an amount that is effective for treating metabolic syndrome.

Another aspect of the invention that is of particular interest relates to a method of treating atherosclerosis, dyslipidemias, diabetes, metabolic syndrome or a related condition in a human patient in need of such treatment, comprising administering to the patient a compound of formula I or a pharmaceutically acceptable salt or solvate thereof and a DP receptor antagonist, said combination being administered in an amount that is effective to treat atherosclerosis, dyslipidemia, diabetes or a related condition in the absence of substantial flushing.

Another aspect of the invention that is of particular interest relates to the methods described above wherein the DP receptor antagonist is selected from the group consisting of compounds A through AJ and the pharmaceutically acceptable salts and solvates thereof.

Moreover, the nicotinic acid receptor has been identified and characterized in WO02/084298A2 published on Oct. 24, 2002 and in Soga, T. et al., Tunaru, S. et al. and Wise, A. et al. (citations above).

Numerous DP receptor antagonist compounds have been published and are useful and included in the methods of the present invention. For example, DP receptor antagonists can be obtained in accordance with WO01/79169 published on Oct. 25, 2001, EP 1305286 published on May 2, 2003, WO02/094830 published on Nov. 28, 2002 and WO03/062200 published on Jul. 31, 2003. Compound AB can be synthesized in accordance with the description set forth in WOO I/66520A1 published on Sep. 13, 2001; Compound AC can be synthesized in accordance with the description set forth in WO03/022814A1 published on Mar. 20, 2003, and Compounds AD and AE can be synthesized in accordance with the description set forth in WO03/078409 published on Sep. 25, 2003. Other representative DP antagonist compounds used in the present invention can be synthesized in accordance with the examples provided below.

### Methods of Synthesis for Compounds of Formula I

Compounds of Formula I have been prepared by the following representative reaction schemes. It is understood that similar reagents, conditions or other synthetic approaches to these structure classes are conceivable to one skilled in the art of organic synthesis. Therefore these reaction schemes should not be construed as limiting the scope of the invention. All substituents are as defined above unless indicated otherwise.

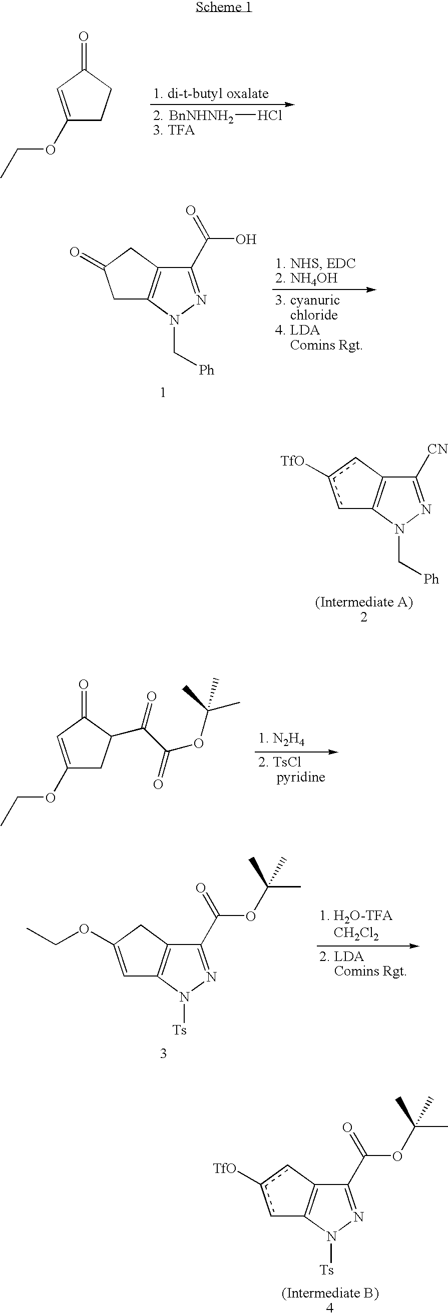

Compounds of Formula I, where n=1, can be prepared by gaining access to the common intermediates, 2 and 4, as shown in Scheme 1. Thus, 3-ethoxy-cyclopentenone can be acylated with a dialkyloxalate, the beta-diketone cyclized with benzylhydrazine-HCl accompanied by concomitant enol ether hydrolysis, and the ester cleaved to provide ketoacid 1. This acid can be transformed into a primary carboxamide, dehydrated to its nitrile, and the ketone converted to its enol triflate by methods known to those skilled in the art, providing common intermediate 2. Alternatively, hydrazine can be used in place of benzylhydrazine-HCl, and the resultant pyrazole can be protected as its N-toluenesulfonamide 3. The ethyl enol ether can be selectively hydrolyzed under acidic conditions, and the resultant ketone converted to its enol triflate by methods known to those skilled in the art, providing common intermediate 4.

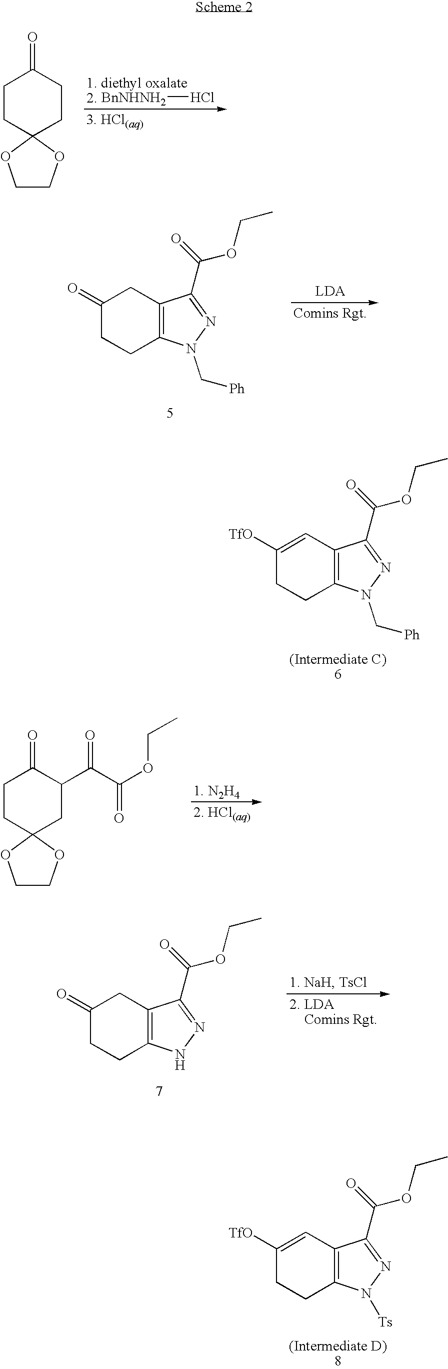

Compounds of Formula I, where n=2, can be prepared by gaining access to the common intermediates, 6 and 8, as shown in Scheme 2. Thus, cyclohexane-1,4-dione mono-ketal can be acylated with a dialkyloxalate, the beta-diketone cyclized with benzylhydrazine-HCl, and the ketal hydrolyzed to ketoester 5. This ketone can then be converted to its enol triflate by methods known to those skilled in the art, providing common intermediate 6. Alternatively, hydrazine can be used in place of benzylhydrazine-HCl, and the ketal hydrolyzed to ketoester 7. The resultant pyrazole 7 can be protected as its N-toluenesulfonamide, and the ketone converted to its enol triflate by methods known to those skilled in the art, providing common intermediate 8.

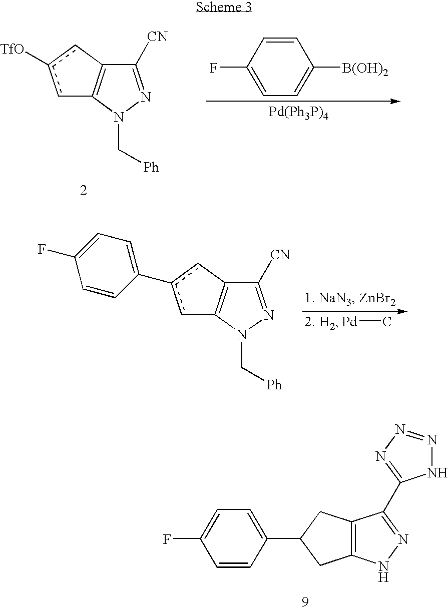

Common intermediate 2 can be converted to compounds such as 9 via Suzuki coupling, which installs the C5 moiety, followed by tetrazole ring synthesis, and a concomitant reduction of the olefin with debenzylation under hydrogenation conditions (Scheme 3).

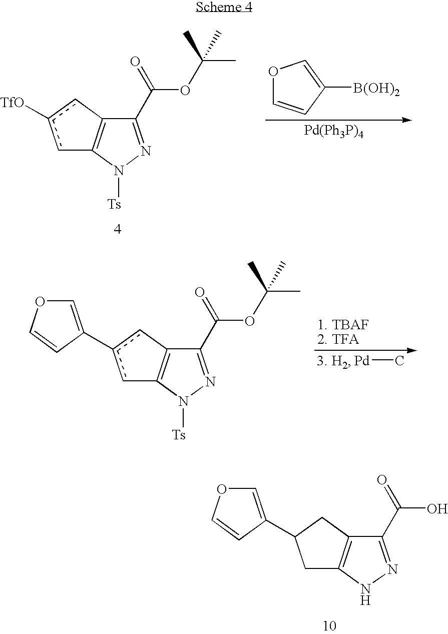

Common intermediate 4 can also be converted to compounds such as 10 via Suzuki coupling, to install the C5 moiety, followed by sequential tosyl and ester cleavage, and a reduction of the olefin under hydrogenation conditions (Scheme 4).

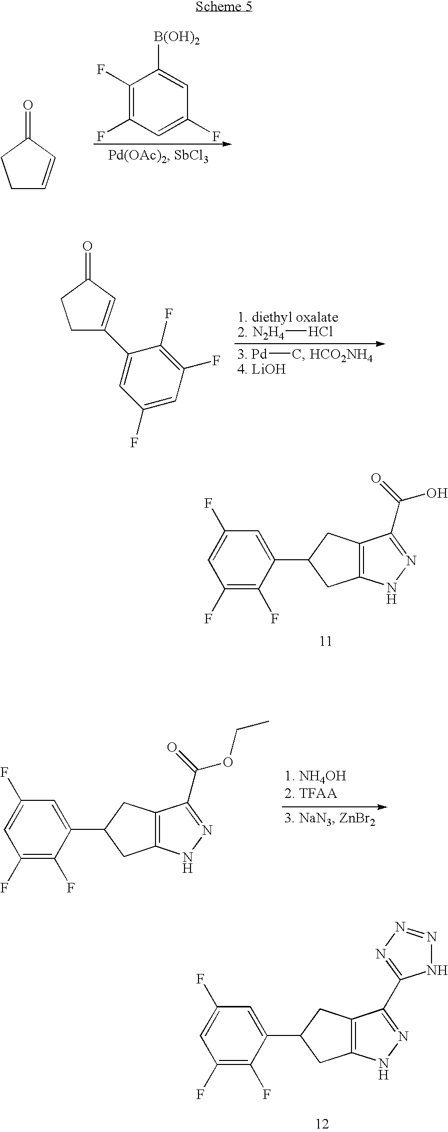

Instead of using common intermediates 2 and 4, compounds such as 11 can also be prepared directly from cyclopentenone as shown in Scheme 5. A modified Suzuki coupling allows the 1,4-addition of a boronic acid, followed by the typical pyrazole ring formation, transfer hydrogenation, and saponification to provide acid 11. Prior to the saponification toward acid 11, the ethyl ester can be converted to a primary carboxamide, dehydrated to its nitrile, and subjected to a tetrazole ring synthesis, as in the preparation of 12.

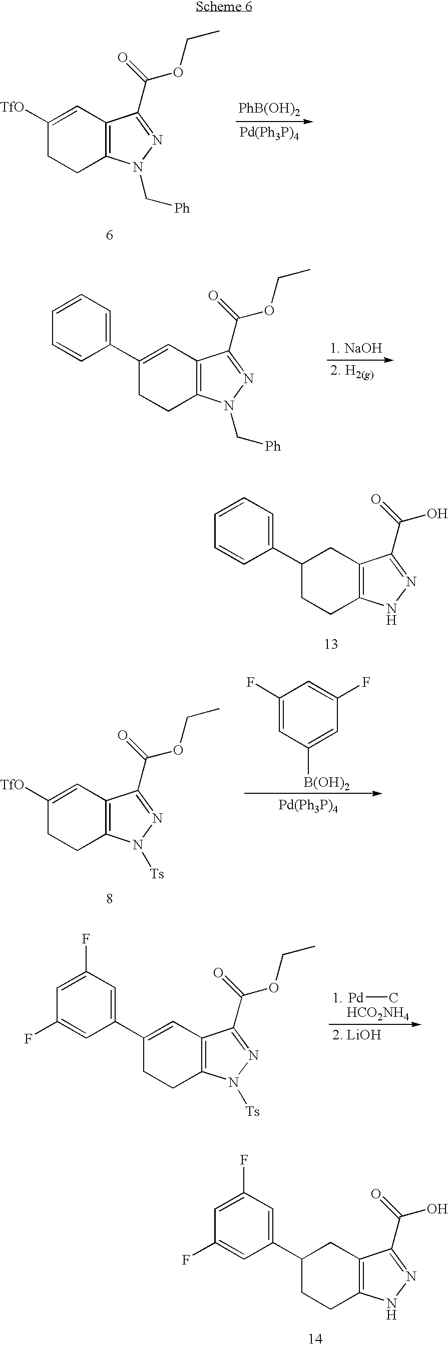

Common intermediate 6 can be converted to compounds such as 13 via Suzuki coupling, to install the C5 moiety, followed by concomitant olefin reduction and benzyl cleavage, and saponification of the ethyl ester (Scheme 6). Alternatively, common intermediate 8 can be used in this reaction sequence to generate compounds such as 14.

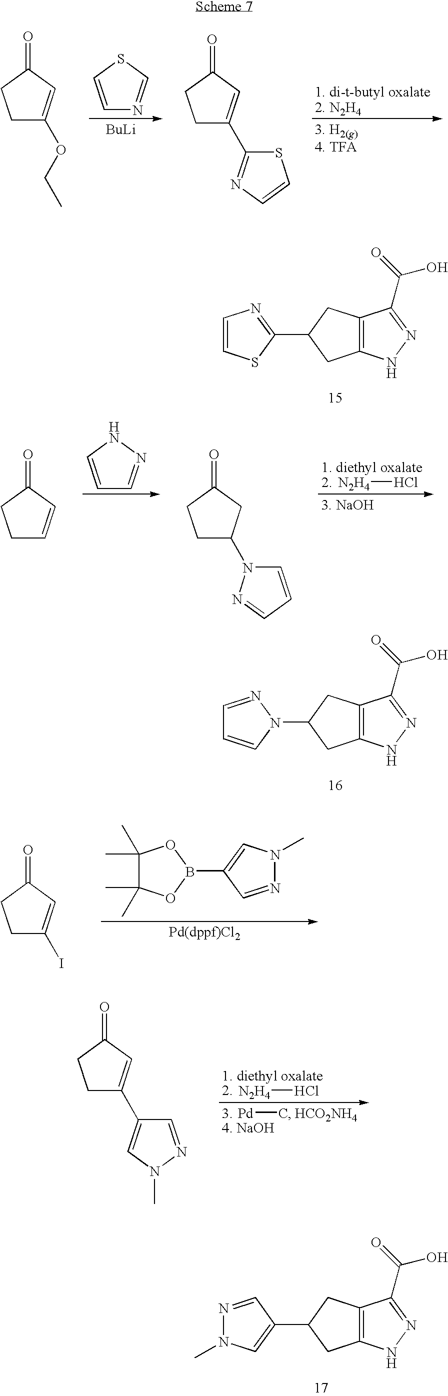

Shown in Scheme 7 are various strategies to incorporate heterocycles at the C5 position of Formula I. For example, deprotonation of a given heterocycle such as thiazole, can generate an anion for the 1,2-addition to 3-ethoxy-cyclopentenone, followed by rearrangement to the beta-substituted enone. Subsequent transformations similar to the schemes above can be used to generate the thiazole derivative 15. Cyclopentenone can also react with nitrogen nucleophiles to form 1,4-addition products with heterocycles such as pyrazole. Subsequent transformations similar to the schemes above can be used to generate the pyrazole derivative 16. Also, 3-iodo-cyclopentenone may participate in a Suzuki coupling with heterocyclic boronate esters, such as the N-methylpyrazole boronate shown in Scheme 7. Again, subsequent transformations similar to the schemes above can be used to generate the N-methylpyrazole derivative 17.

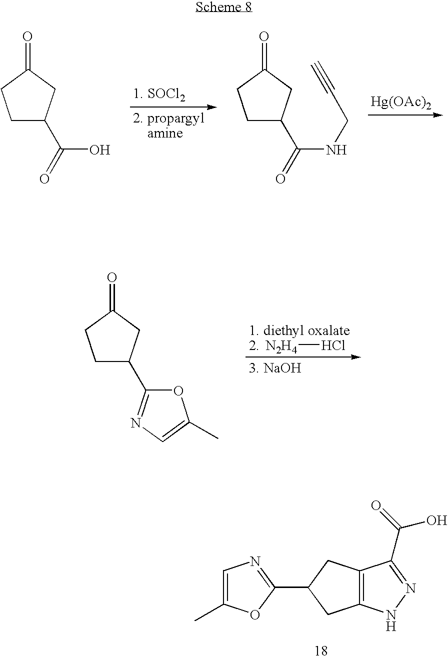

Scheme 8 displays a route toward oxazole derivative 18. The propargyl amide, generated from 3-carboxy-cyclopentanone, can be cyclized with mercury salts to the cyclopentanone oxazole (J. Med. Chem. 1990, 33, 1128), followed by the subsequent transformations described in the schemes above to afford oxazole derivative 18.

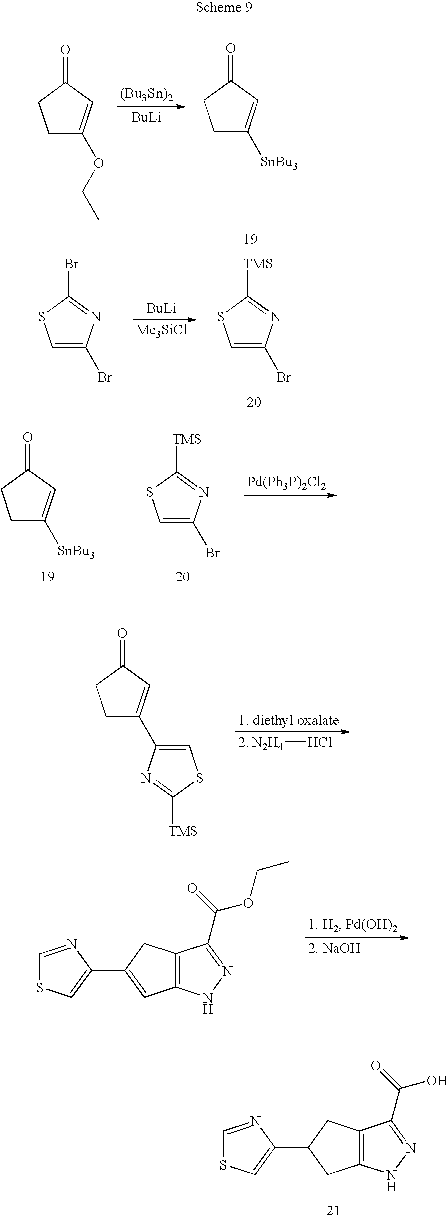

Scheme 9 displays a route toward thiazole regio-isomer 21. Stannylation of 3-ethoxy-cyclopentenone can generate intermediate 19, and conversion of 2,4-dibromo-thiazole to 20 allows for regio-control in the subsequent Stille coupling. Thus, coupling of 19 and 20 provides the beta-substituted cyclopentenone intermediate, which following the subsequent transformations described in the schemes above, can afford thiazole derivative 21.

The various organic group transformations and protecting groups utilized herein can be performed by a number of procedures other than those described above. References for other synthetic procedures that can be utilized for the preparation of intermediates or compounds disclosed herein can be found in, for example, M. B. Smith, J. March Advanced Organic Chemistry, 5^{th} Edition, Wiley-Interscience (2001); R. C. Larock Comprehensive Organic Transformations, A Guide to Functional Group Preparations, 2^{nd} Edition, VCH Publishers, Inc. (1999); T. L. Gilchrist Heterocyclic Chemistty, 3^{rd} Edition, Addison Wesley Longman Ltd. (1997); J. A. Joule, K. Mills, G. F. Smith Heterocyclic Chemistry, 3^{rd} Edition, Stanley Thomes Ltd. (1998); G. R. Newkome, W. W. Paudler Contemporary Heterocyclic Chemistry, John Wiley and Sons (1982); or Wuts, P. G. M.; Greene, T. W.; Protective Groups in Organic Synthesis, 3^{rd} Edition, John Wiley and Sons, (1999), all six incorporated herein by reference in their entirety.

Examples of compounds described by Formula I, are shown below in Table 1. It is understood that the examples in Table 1 are representative, and not intended to be limiting in any manner.

TABLE 1

| | |
|---|---|
| | COMPOUND 1 |
| 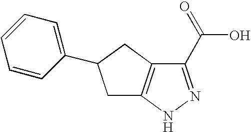 | |
| | COMPOUND 2 |
| 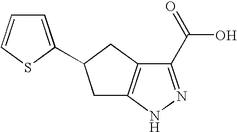 | |
| | COMPOUND 3 |
| 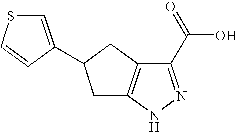 | |
| | COMPOUND 4 |
| 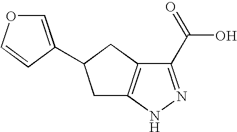 | |
| | COMPOUND 5 |
| 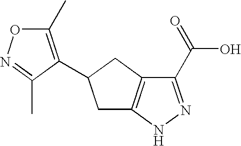 | |
| | COMPOUND 6 |
| 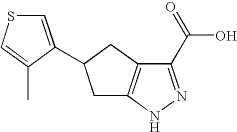 | |
| | COMPOUND 7 |
| 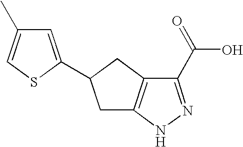 | |
| | COMPOUND 8 |
| 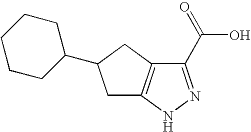 | |
| | COMPOUND 9 |
| 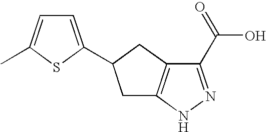 | |
| | COMPOUND 10 |
| 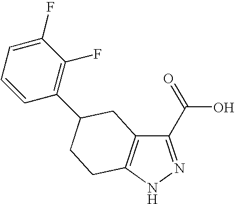 | |
| | COMPOUND 11 |
| 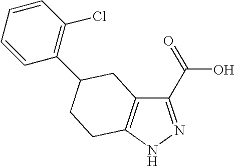 | |
| | COMPOUND 12 |
| 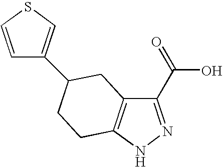 | |
| | COMPOUND 13 |
| 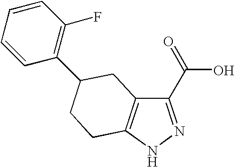 | |
| | COMPOUND 14 |
| 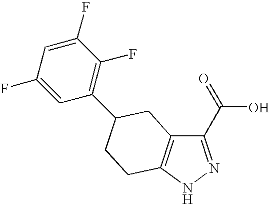 | |
| | COMPOUND 15 |
| 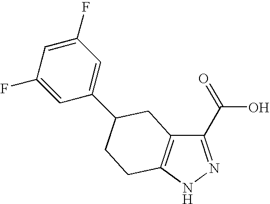 | |
| | COMPOUND 16 |
| 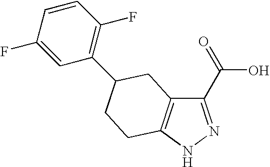 | |
| | COMPOUND 17 |
| 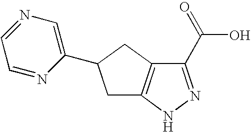 | |
| | COMPOUND 18 |
| 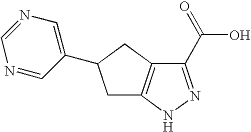 | |
| | COMPOUND 19 |
| 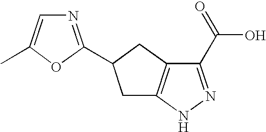 | |
| | COMPOUND 20 |
| 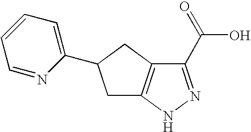 | |
| | COMPOUND 21 |
| 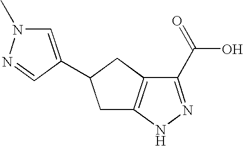 | |
| | COMPOUND 22 |
| 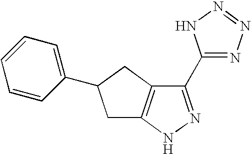 | |
| | COMPOUND 23 |
| 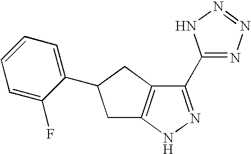 | |
| | COMPOUND 24 |
| 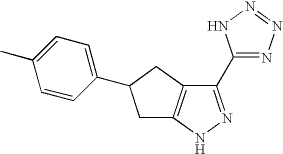 | |
| | COMPOUND 25 |
| 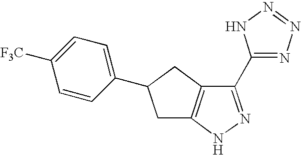 | |
| | COMPOUND 26 |
| 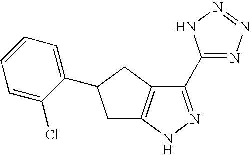 | |
| | COMPOUND 27 |
| 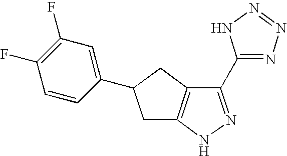 | |
| | COMPOUND 28 |
| 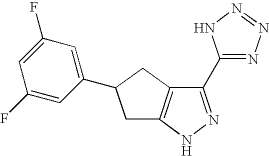 | |
| | COMPOUND 29 |
| 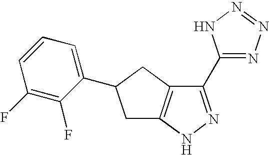 | |
| | COMPOUND 30 |
| 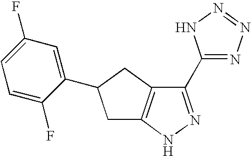 | |
| | COMPOUND 31 |
| 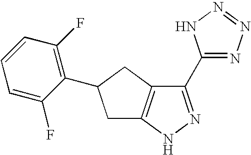 | |
| | COMPOUND 32 |
| 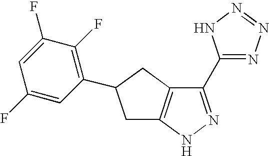 | |
| | COMPOUND 33 |
| 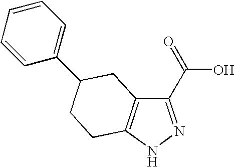 | |
| | COMPOUND 34 |
| 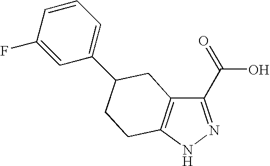 | |
| | COMPOUND 35 |
| 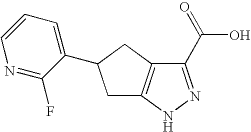 | |
| | COMPOUND 36 |
| 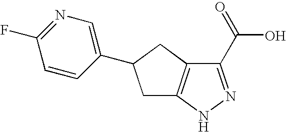 | |
| | COMPOUND 37 |
| 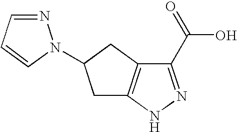 | |
| | COMPOUND 38 |
| 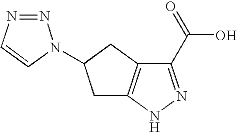 | |
| | COMPOUND 39 |
| 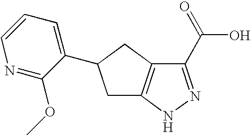 | |
| | COMPOUND 40 |
| 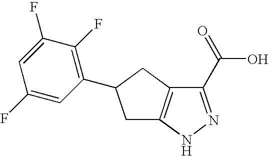 | |
| | COMPOUND 41 |
| 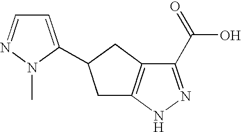 | |
| | COMPOUND 42 |
| 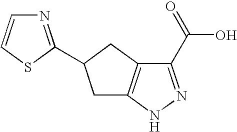 | |
| | COMPOUND 43 |
| 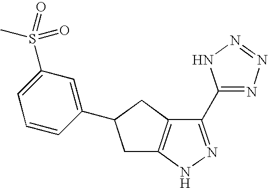 | |
| | COMPOUND 44 |
| 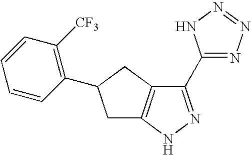 | |
| | COMPOUND 45 |
| 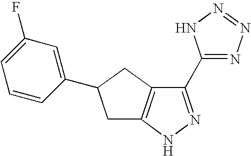 | |
| | COMPOUND 46 |
| 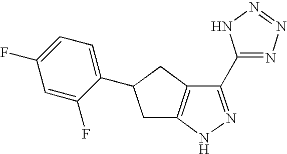 | |
| | COMPOUND 47 |
| 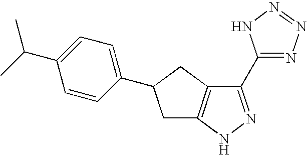 | |
| | COMPOUND 48 |
| 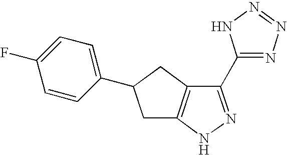 | |
| | COMPOUND 49 |
| 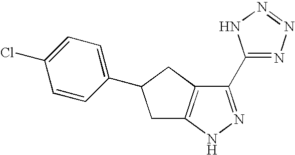 | |
| | COMPOUND 50 |
| 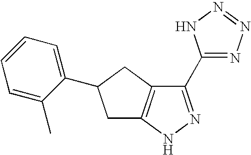 | |
| | COMPOUND 51 |
| 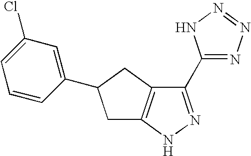 | |
| | COMPOUND 52 |
| 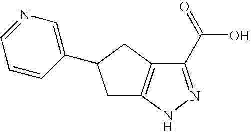 | |
| | COMPOUND 53 |
| 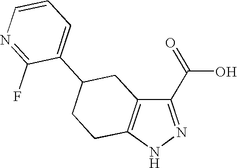 | |
| | COMPOUND 54 |
| 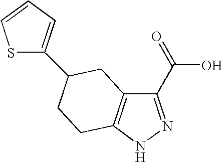 | |
| | COMPOUND 55 |
| 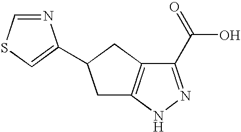 | |

Pharmaceutically acceptable salts and solvates thereof are included as well.

Compounds of Formula I, have one or more chiral centers, and therefore will exist as enantiomers or diastereomers. Formula I and the formulae described throughout this invention are intended to represent all such enantiomers, diastereomers and mixtures thereof, including racemates, unless stated or shown otherwise. All such isomeric forms are included.

Moreover, chiral compounds possessing one stereocenter of general Formula I, may be resolved into their enantiomers in the presence of a chiral environment using methods known to those skilled in the art. Chiral compounds possessing more than one stereocenter may be separated into their diastereomers in an achiral environment on the basis of their physical properties using methods known to those skilled in the art. Single diastereomers that are obtained in racemic form may be resolved into their enantiomers as described above.

If desired, racemic mixtures of compounds may be separated so that individual enantiomers are isolated. This separation can be carried out by methods well known in the art, such as the coupling of a racemic mixture of compounds of Formula I to an enantiomerically pure compound to form a diastereomeric mixture, which is then separated into individual diastereomers by standard methods, such as fractional crystallization or chromatography. The coupling reaction can be, but is not limited to, the formation of salts using an enantiomerically pure acid or base. The diasteromeric derivatives may then be converted to substantially pure enantiomers by cleaving the added chiral residue from the diastereomeric compound. The racemic mixture of the compounds of Formula I can also be separated directly by chromatographic methods utilizing chiral stationary phases, the methods of which are well known in the art. Additional methods for the resolution of optical isomers can be used, and will be apparent to the average worker skilled in the art. Such methods include, but are not limited to, those discussed by J. Jaques, A. Collet, and S. Wilen in “Enantiomers, Racemates, and Resolutions”, John Wiley and Sons, New York (1981). Alternatively, enantiomers of compounds of the general Formula I may be obtained by asymmetric stereoselective synthesis using optically pure starting materials or reagents, obtained either from the chiral pool or from synthetic sources. Such asymmetric synthetic methods include, but are not limited to, those discussed by J. D. Morrison, Ed. Asymmetric Synthesis; Academic Press: Orlando, Volume 5, (1985).

The compounds described herein may also exist as tautomers, which have different points of attachment for hydrogen accompanied by one or more double bond shifts. For example, a ketone and its enol form are keto-enol tautomers. Or for example, the proton of a pyrazole may reside on either of the two nitrogens within the heterocyclic ring. Formula I and the formulae described herein are intended to represent all individual tautomers and mixtures thereof, unless stated otherwise.

### REPRESENTATIVE EXAMPLES

The following examples are provided to more fully illustrate the present invention, and shall not be construed as limiting the scope in any manner. Unless stated otherwise:

(i) all operations were carried out at room or ambient temperature, that is, at a temperature in the range 18-25° C.;

(ii) evaporation of solvent was carried out using a rotary evaporator under reduced pressure (4.5-30 mmHg) with a bath temperature of up to 50° C.;

(iii) the course of reactions was followed by thin layer chromatography (TLC) and/or tandem high performance liquid chromatography (HPLC) followed by mass spectroscopy (MS), herein termed LCMS, and any reaction times are given for illustration only;

(iv) yields, if given, are for illustration only;

(v) the structure of all final compounds was assured by at least one of the following techniques: MS or proton nuclear magnetic resonance (1H NMR) spectrometry, and the purity was assured by at least one of the following techniques: TLC or HPLC;

(vi) 1H NMR spectra were recorded on either a Varian Unity or a Varian Inova instrument at 500 or 600 MHz using the indicated solvent; when line-listed, NMR data is in the form of delta values for major diagnostic protons, given in parts per million (ppm) relative to residual solvent peaks (multiplicity and number of hydrogens); conventional abbreviations used for signal shape are: s. singlet; d. doublet (apparent); t. triplet (apparent); m. multiplet; br. broad; etc.;

(vii) MS data were recorded on a Waters Micromass unit, interfaced with a Hewlett-Packard (Agilent 1100) HPLC instrument, and operating on MassLynx/OpenLynx software; electrospray ionization was used with positive (ES+) or negative ion (ES−) detection; the method for LCMS ES+ was 1-2 mL/min, 10-95% B linear gradient over 5.5 min (B=0.05% TFA-acetonitrile, A=0.05% TFA-water), and the method for LCMS ES− was 1-2 mL/min, 10-95% B linear gradient over 5.5 min (B=0.1% formic acid−acetonitrile, A=0.1% formic acid−water), Waters XTerra C18-3.5 um-50×3.0 mmID and diode array detection;

(viii) the purification of compounds by preparative reverse phase HPLC (RPHPLC) was conducted on either a Waters Symmetry Prep C18-5 um-30×100 mmID, or a Waters Atlantis Prep dC18-5 um-20×100 mmID; 20 mL/min, 10-100% B linear gradient over 15 min (B=0.05% TFA-acetonitrile, A=0.05% TFA-water), and diode array or 254 wavelength detection;

(ix) the automated purification of compounds by preparative reverse phase HPLC was performed on a Gilson system using a YMC-Pack Pro C18 column (150×20 mm i.d.) eluting at 20 mL/min with 0-50% acetonitrile in water (0.1% TFA);

(x) the purification of compounds by preparative thin layer chromatography (PTLC) was conducted on 20×20 cm glass prep plates coated with silica gel, or centrifugal chromatography on a chromatotron using glass rotors coated with silica gel, both commercially available from Analtech;

(xi) column chromatography was carried out on a glass silica gel column using Kieselgel 60, 0.063-0.200 mm (Merck), or a Biotage cartridge system;

(xii) microwave irradiations were conducted using the Smith Synthesizer (Personal Chemistry);

(xiii) chemical symbols have their usual meanings; the following abbreviations have also been used v (volume), w (weight), b.p. (boiling point), m.p. (melting point), L (litre(s)), mL (millilitres), g (gram(s)), mg (milligrams(s)), mol (moles), mmol (millimoles), eq or equiv (equivalent(s)), IC50 (molar concentration which results in 50% of maximum possible inhibition), EC50 (molar concentration which results in 50% of maximum possible efficacy), uM (micromolar), nM (nanomolar); (xiv) definitions of acronyms are as follows:

THF is tetrahydrofuran;

DME is 1,2-dimethoxyethane;

DMF is dimethylformamide;

DCM is dichloromethane (methylene chloride);

TFA is trifluoroacetic acid;

TBAF is tetrabutylammonium fluoride;

TFAA is trifluoroacetic anhydride;

LDA is lithium diisopropyl amide;

EDC(I) is 1-ethyl-3-(3-dimethylaminopropyl)carbodiimide hydrochloride;

NHS is N-hydroxy succinimide;

TsCl is toluenesulfonyl (tosyl) chloride;

dppf is 1,1′-bis(diphenylphosphino)ferrocene;

UV is ultraviolet;

(ee) is enantiomeric excess.

### Intermediate A

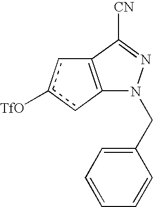

To a solution, of 3-ethoxy cyclopentenone (2.12 g, 16.82 mmol) in anhydrous THF (40 mL) cooled to −78° C. under a nitrogen atmosphere was added lithium diisopropyl amide (12 mL, 24 mmol, 2.0 M in THF). After 15 minutes, a solution of di-tert-butyl dioxalate (3.73 g, 18.5 mmol) in THF (15 mL) was added. The reaction mixture was stirred at −78° C. for 15 minutes and then warmed to −20° C. and stirred for an additional 15 minutes. The reaction was quenched with 1N HCl (40 mL) and extracted with ethyl acetate (3×). The organic layer was washed with brine, dried over anhydrous Na₂SO₄, filtered and concentrated in vacuo. The residue was purified by flash chromatography (SiO₂) using 35% ethyl acetate-hexanes to give the desired product as an off-white solid. To a solution of this diketone (2.15 g, 8.45 mmol) in ethanol (100 mL) was added benzyl hydrazine hydrochloride (1.8 g, 9.22 mmol) and HOAc (10 mL). The reaction mixture was stirred at room temperature for 16 hours and then refluxed at 70° C. for 30 minutes. The reaction was cooled to room temperature and concentrated in vacuo. The residue was dissolved in ethyl acetate and washed with water, saturated NaHCO₃, and brine. The organic layer was dried over anhydrous Na₂SO₄ filtered and concentrated in vacuo. The residue was purified by flash chromatography (SiO₂) using 30% ethyl acetate-hexanes to give the desired product as a brown oil. To a solution of this tert-butyl ester (1.64 g, 5.25 mmol) in dichloromethane (20 mL) was added trifluoroacetic acid (20 mL) and the resulting solution stirred at room temperature for 4 hours. The reaction mixture was concentrated in vacuo and azeotroped with toluene (3×). This material was used in the next step without any further purification. To a solution of this acid (1.34 g, 5.25 mmol) in CH₂Cl₂(50 mL) was added N-hydroxy succinimide (1.21 g, 10.5 mmol) followed by EDC (2.01 g, 10.5 mmol). After stirring at room temperature for 18 hours, the reaction mixture was concentrated in vacuo. The residue was diluted with ethyl acetate (200 mL), washed with saturated NaHCO₃, solution and brine. The organic layer was dried over anhydrous Na₂SO₄, filtered and concentrated in vacuo. A yellow solid was obtained. To a solution of this activated ester (2.0 g, 5.25 mmol) in 1,4-dioxane (50 En) was added NH₄OH (14.8 N, 10.0 eq., 3.53 mL). A precipitate formed immediately. After stirring at room temperature for 15 minutes the reaction mixture was filtered through a fritted funnel and the precipitate washed with 1,4-dioxane. The filtrate was concentrated in vacuo to give a solid. To a solution of this carboxamide (5.25 mmol) in DMF (60 mL) was added cyanuric chloride (3.12 g, 17 mmol) in three portions. After 30 minutes at room temperature, the reaction was quenched with water and extracted with ethyl acetate (2×). The organic layer was washed with water, brine and dried over anhydrous Na₂SO₄, filtered, and concentrated in vacuo. The residue was purified by flash chromatography (SiO₂) using 30% ethyl acetate-hexanes to give the desired product as a yellow solid. To a solution of this cyano ketone (447 mg, 1.87 mmol) in anhydrous THF (14 mL) at −78° C. was added a solution of freshly prepared lithium diisopropyl amide (1.89 mmol) in THF (6 mL). After stirring the reaction at −78° C. for 30 minutes 2-[N,N-Bis(trifluoromethylsulfonyl)amine]-5-chloropyridine (Comins Reagent, 1.4 g, 3.6 mmol) was added. The reaction was warmed to −20° C. and stirred for 3 hours. The reaction was quenched with saturated NH₄Cl solution, and the resulting mixture was extracted with ethyl acetate, washed with 1N HCl solution, saturated NaHCO₃ solution and dried over anhydrous Na₂SO₄. The solution was filtered and concentrated in vacuo. The residue was purified on the chromatotron using a 2000-micron rotor (SiO₂) and 5% ethyl acetate-hexanes as eluant to afford the desired product as a 2:1 mixture of double bond regio-isomers.

¹H NMR (500 MHz, CDCl₃): (major isomer) δ 7.45-7.3 (m, 5H), 6.06 (bt, 1H), 5.41 (s, 2H), 3.56 (bd, 2H); (minor isomer) δ 7.45-7.3 (m, 5H), 6.63 (bt, 1H), 5.39 (s, 2H), 3.18 (bd, 2H); LCMS m/z 370 (M+H).

### Intermediate B

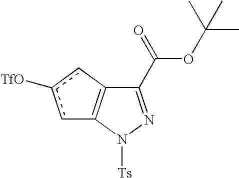

To a solution of the tert-butyl(4-ethoxy-2-oxocyclopent-3-ene-1yl)(oxo)acetate (4.0 g, 15.7 mmol) (Intermediate A, step 1) in ethanol (90 mL) was added hydrazine (0.54 mL, 17.3 mmol). After 5 minutes, glacial acetic acid (10 μL) was added. The resulting solution was heated at 70° C. for 1 hour. The reaction was cooled to room temperature and concentrated in vacuo. The resulting oil was purified by flash chromatography using Biotage flash 60M cartridge using 1:1 ethyl acetate-hexanes as eluant to give the desired compound as a white solid. To a solution of this free pyrazole (2.78, 11 mmol) in DCM (100 mL) was added pyridine (2.69 mL, 33.3 mmol) followed by tosyl chloride (3.17 g, 16.51 mmol). The reaction was stirred at room temperature for 4 hours and quenched with 1N HCl. The resulting mixture was extracted with DCM. The organic layer was washed with saturated NaHCO₃ solution, brine and dried over anhydrous Na₂SO₄. It was filtered and concentrated in vacuo. The crude material was purified by flash chromatography (SiO₂) using 25% ethyl acetate-hexanes to give the desired compound as a white solid. To a solution of this enol ether (1.15 g, 2.84 mmol) in DCM (19 mL) was added TFA (1 mL) and water (0.2 mL). After stirring for 20 minutes at room temperature, the reaction was quenched by adding saturated NaHCO₃ solution. The resulting mixture was extracted with DCM (3×). The organic layer was washed with saturated NaHCO₃, brine and dried over anhydrous Na₂SO₄. The solution was filtered and concentrated in vacuo. The crude material was purified by flash chromatography using 30% ethyl acetate-hexanes to give the desired compound as a white solid. To a solution of this ketone (595 mg, 1.58 mmol) in anhydrous THF (20 mL) at −78° C. under a nitrogen atmosphere was added a solution of lithium diisopropyl amide (0.79 mL, 1.58 mmol, 2.0 M solution in THF). After stirring the reaction at −78° C. for 5 minutes 2-[N,N-Bis(trifluoromethylsulfonyl)amine]-5-chloropyridine (Comins Reagent, 0.745 g, 1.89 mmol) was added. The reaction was warmed to room temperature and quenched with saturated 1N HCl solution. The resulting mixture was extracted with ethyl acetate, washed with saturated NaHCO₃ solution and dried over anhydrous Na₂SO₄. The solution was filtered and concentrated in vacuo. The residue was purified by flash chromatography (SiO₂) using 15% ethyl acetate-hexanes to give the desired product

LCMS m/z 509 (M+H).

### Intermediate C

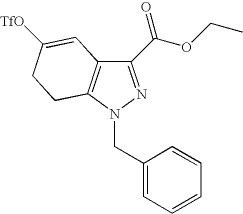

To a solution of cyclohexane 1,4-dione mono-ethylene ketal (5 g, 32 mmol) and diethyl oxalate (5.6 g, 38.4 mmol) in ethanol (100 mL) was added potassium-t-butoxide (38 mL, 1.0 M in THF). After 1 hour, benzyl hydrazine hydrochloride (6.9 g, 35.2 mmol) was added and the resulting reaction mixture was stirred at room temperature for 16 hours. The reaction mixture was concentrated in vacuo and diluted with ethyl acetate. The organic layer was washed with water, brine and dried over anhydrous Na₂SO₄. The reaction mixture was filtered, concentrated in vacuo and purified by flash chromatography to give the desired compound. To a solution of this ketal (3.0 g, 8.76 mmol) in THF (30 mL) was added ethanol (50 mL) followed by 3N HCl (20 mL). After stirring at 50° C. overnight the reaction was concentrated in vacuo. The residue was diluted with ethyl acetate and washed with saturated NaHCO₃. The organic layer was concentrated in vacuo and purified by flash chromatography (Horizon) using a gradient of 0-60% ethyl acetate-hexanes. This ketone was converted to its enol triflate following a similar procedure as described for Intermediates A and B.

LCMS m/z 231 (M+H).

### Intermediate D

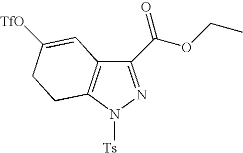

To a solution of cyclohexane-1,4-dione mono ethylene ketal (10.75 g, 68.83 mmol) in anhydrous THF cooled to −78° C. under a nitrogen atmosphere was added LDA (37 mL, 2.0 M solution). After stirring for 15 minutes, diethyl oxalate (10.3 mL, 74 mmol) was added. The reaction was warmed to room temperature and stirred for 16 hours. The reaction mixture was quenched with 1N HCl and the resulting mixture was extracted with ethyl acetate, washed with brine and dried over anhydrous Na₂SO₄. The solution was filtered and concentrated in vacuo. The residue was purified by flash chromatography (Biotage-Horizon) using a gradient of 0 to 100% ethyl acetate hexanes. To a solution of this diketone (10.5 g, 40.98 mmol) in ethanol (300 mL) was added hydrazine hydrate (1.4 mL, 45.07 mmol) and glacial acetic acid (30 mL). The resulting reaction mixture was heated to 65° C. for 1.5 hours. The reaction mixture was concentrated in vacuo. The residue was dissolved in ethyl acetate, washed with saturated NaHCO₃, brine, and dried over anhydrous Na₂SO₄. The solution was filtered and concentrated in vacuo. The residue was purified by flash chromatography (Biotage 40 M) using 60% ethyl acetate-hexanes as solvent to give the desired compound as a yellow foam. To a solution of this ketal (6.76 g, 26.8 mmol) in 2:1 EtOH/THF was added 3N HCl (50 mL). The resulting reaction mixture was heated to 60° C. for 48 hours. The reaction was quenched by the addition of saturated NaHCO₃ solution. The organic solvent was removed in vacuo. The residue was extracted with ethyl acetate. The organic layer was washed with brine, dried over anhydrous Na₂SO₄, filtered and concentrated in vacuo to give orange oil. This material was purified by flash chromatography (Biotage flash 40M) using 5% ethyl acetate-hexanes to give the desired product as a white solid. To a solution of this free pyrazole (0.47 g, 2.26 mmol) in anhydrous THF cooled to 0° C. was added sodium hydride (0.108 g, 2.7 mmol). After 30 minutes tosyl chloride was added (0.452 g, 2.37 mmol). The reaction was stirred at room temperature for 1 hour and then quenched by the addition of 1N HCl. The resulting mixture was diluted with water and extracted with ethyl acetate (2×). The organic layer was washed with brine, dried over anhydrous Na₂SO₄, filtered and concentrated in vacuo. The residue was purified by Prep TLC using 50% ethyl acetate-hexanes to give the desired compound as a white solid. To a solution of this ketone (0.941 mg, 2.6 mmol) in anhydrous THF (20 mL) cooled to −70° C. under a N₂ atmosphere was added LDA (1.3 mL, 2.0 M solution). After stirring at −78° C. for 30 minutes 2-[N,N-Bis(trifluoromethylsulfonyl)amine]-5-chloropyridine (Comins Reagent, 1.55 g, 3.9 mmol) was added. The reaction was slowly warmed to room temperature and stirred for 4 hours. It was quenched by the addition of 1N HCl. The resulting mixture was extracted with ethyl acetate. The organic layer was washed with brine, dried over anhydrous Na₂SO₄, filtered and concentrated in vacuo. The residue was purified by flash chromatography (Biotage 40M) using 15% ethyl acetate-hexanes to give the desired compound as a white solid.

LCMS m/z 494.9 (M+H).

### Example 1

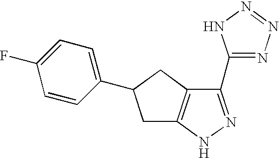

Intermediate A (50 mg, 0.14 mmol) was combined with 1.1 equivalents of 4-fluorophenyl boronic acid, triethylamine (28 mg, 0.28 mmol) and Pd(Ph₃P)₄ (15 mg, 10% catalyst) in dioxane (1 mL). The reaction mixture was heated in a microwave reactor at 100° C. (100 Watts) for 10 min, partitioned with 1M NaOH, brine, the organic phase dried over anhydrous sodium sulfate, and concentrated in vacuo. The crude product was purified by preparative centrifugal chromatography (SiO₂, hexane-EtOAc) to afford the cyano benzyl olefinic intermediate. This material (25 mg, 0.079 mmol) was diluted into water (1 mL) and isopropanol (2 mL), and the turbid mixture treated with sodium azide (10 mg, 0.16 mmol) and ZnBr₂ (26 mg, 0.12 mmol). The heterogeneous reaction mixture was refluxed for 16 h, cooled to room temperature, acidified to pH 2 with conc. HCl, extracted with EtOAc, and the organic phase dried over anhydrous sodium sulfate, and concentrated in vacuo. The benzyl-protected olefinic intermediate (17 mg, 0.047 mmol) was then diluted into methanol (2 mL), and the turbid mixture brought to homogeneity with the dropwise addition of concentrated HCl. Catalytic Pd—C (10% by weight) was added, and the reaction mixture stirred vigorously under 1 atmosphere of hydrogen gas (balloon) for 48 h. The reaction mixture was filtered and concentrated in vacuo to provide the desired product.

¹H NMR (CD₃OD, 500 MHz) δ 7.37 (m, 2H), 7.05 (m, 2H), 4.28 (m, 1H), 3.33 (m, 2H), 2.94 (m, 2H); LCMS m/z 271 (M+H).

### Examples 2-19

The following compounds were prepared under conditions similar to those described in EXAMPLE 1 above and illustrated in Scheme 3. All examples, except EXAMPLE 2, utilized a microwave reactor for facilitation of the palladium coupling reaction. EXAMPLE 2 was conducted under thermal conditions (85° C., 2 h), with the use of potassium phosphate as base.

| | | MS |
|---|---|---|
| | | m/z |
| EXAMPLE | STRUCTURE | (M + H) |
| 2 | 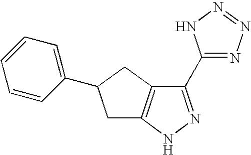 | 253 |
| 3 | 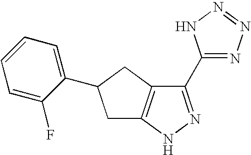 | 271 |
| 4 | 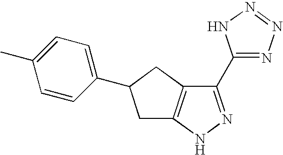 | 267 |
| 5 | 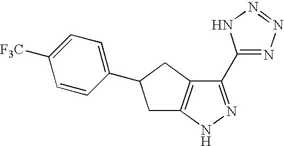 | 321 |
| 6 | 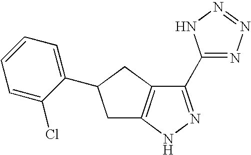 | 287 |
| 7 | 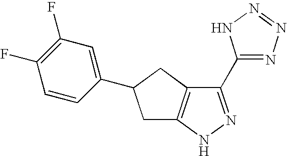 | 289 |
| 8 | 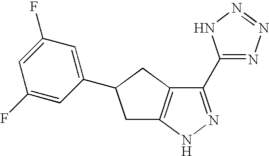 | 289 |
| 9 | 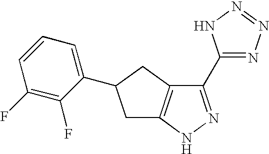 | 289 |
| 10 | 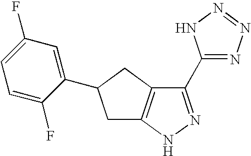 | 289 |
| 11 | 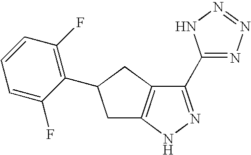 | 289 |
| 12 | 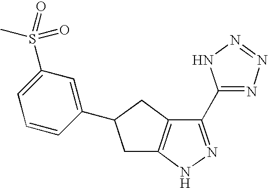 | 331 |
| 13 | 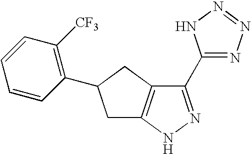 | 321 |
| 14 | 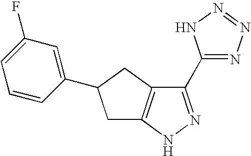 | 271 |
| 15 | 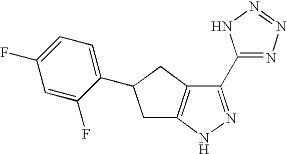 | 289 |
| 16 | 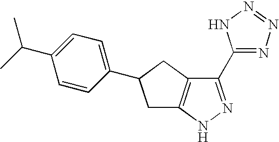 | 295 |
| 17 | 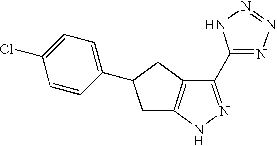 | 287 |
| 18 | 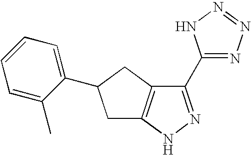 | 267 |
| 19 | 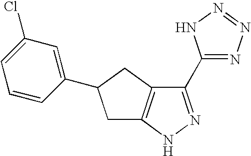 | 287 |

¹H NMR for selected examples:

### Example 2

### (500 MHz, CD₃OD) δ 2.95 (m, 2H), 3.40 (m, 2H), 4.27 (m, 1H), 7.20-7.40 (m, 5H).

### Example 3

### (500 MHz, CD₃OD) δ 2.95 (m, 2H), 3.40 (m, 2H), 4.45 (bd, 1H), 7.05-7.40 (m, 4H).

### Example 4

### (500 MHz, CD₃OD) δ 2.29 (s, 3H), 2.95 (m, 2H), 3.30 (m, 2H), 4.25 (m, 1H), 7.05 (m, 1H), 7.20 (m, 3H).

### Example 5

### (500 MHz, CD₃OD) δ 2.99 (m, 2H), 3.36 (m, 2H), 4.35 (m, 1H), 7.55 (d, 2H), 7.65 (d, 2H).

### Example 6

### (500 MHz, CD₃OD) δ 3.00 (m, 2H), 3.40 (m, 2H), 4.70 (bm, 1H), 7.20-7.60 (m, 4H).

### Example 7

### (500 MHz, CD₃OD) δ 2.90 (bm, 2H), 3.40 (m, 2H), 4.30 (m, 1H), 7.40 (m, 3H).

### Example 8

### (500 MHz, CD₃OD) δ 2.90 (bm, 2H), 3.40 (bm, 2H), 4.30 (bs, 1H), 6.81 (bt, 1H), 6.96 (bd, 2H).

### Example 9

### (500 MHz, CD₃OD) δ 2.95 (m, 2H), 3.40 (m, 2H), 4.40 (m, 1H), 7.00 (m, 1H), 7.10 (m, 2H).

### Example 10

### (500 MHz, CD₃OD) δ 3.05 (m, 2H), 3.20 (m, 2H), 4.64 (m, 1H), 6.99 (t, 2H), 7.31 (m, 1H).

### Example 11

### (500 MHz, CD₃OD) δ 3.05 (m, 2H), 3.21 (m, 2H), 4.64 (m, 1H), 6.99 (bt, 2H), 7.31 (m, 1H).

### Example 12

### (500 MHz, CD₃OD) δ 3.00 (m, 2H), 3.15 (s, 3H), 3.40 (m, 2H), 4.43 (bm, 1H), 7.65 (t, 1H), 7.76 (d, 1H), 7.87 (d, 1H), 7.96 (s, 1H).

### Example 13

### (500 MHz, CD₃OD) δ 3.00 (m, 2H), 3.40 (m, 2H), 4.70 (bm, 1H), 7.40 (m, 1H), 7.60-7.70 (m, 3H).

### Example 15

(500 MHz, CD₃OD) 2 apparent rotamers δ 2.95 (m, 2H), 3.40 (m, 2H), 4.42 (m, 1H), 6.90-7.77 (m, 3H).

### Example 16

### (500 MHz, CD₃OD) δ 1.25 (d, 6H), 2.95 (m, 3H), 3.40 (m, 2H), 4.20 (m, 1H), 7.18 (d, 2H), 7.26 (d, 2H).

### Example 17

### (500 MHz, CD₃OD) δ 2.90 (m, 2H), 3.40 (m, 2H), 4.25 (m, 1H), 7.40 (bq, 4H).

### Example 18

### (500 MHz, CD₃OD) δ 2.40 (s, 3H) 2.90 (m, 2H), 3.30 (m, 2H), 4.51 (m, 1H), 7.20 (m, 3H), 7.30 (d, 1H).

### Example 19

### (500 MHz, CD₃OD) δ 2.95 (m, 2H), 3.40 (m, 2H), 4.30 (bm, 1H), 7.40 (m, 4H).

### Example 20

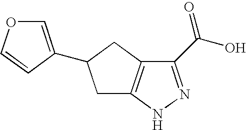

To a solution of Intermediate B (0.05 g, 0.1 mmol) in DME (1.5 mL) was added 3-furyl boronic acid (13.4 mg, 0.12 mmol), triethyl amine (42 μL, 0.2 mmol) and tetrakis triphenyl phosphine palladium (0) (0.01 mmol, 11 mg). The resulting mixture was heated in the microwave for 10 minutes at 100° C. The reaction mixture was directly loaded on the Biotage Quad 3 12 M column and eluted with 15% ethyl acetate hexanes to give the desired compound (36 mg). To a solution of this toluenesulfonamide (36 mg, 0.13 mmol) in THF (1 mL) was added TBAF (100 μL, 1.0 M solution) and the resulting mixture was heated to 80° C. in a sealed tube for 1 hour. The reaction mixture was cooled to room temperature and directly loaded on to the Biotage Quad 3 12M column and eluted with 30% ethyl acetate-hexanes to give the desired compound. To a solution of this tert-butyl ester in DCM (3 mL) was added TFA (3 mL) and the resulting reaction stirred at room temperature for 3 hours. The reaction mixture was concentrated in vacuo and purified on Biotage Quad 3 using 50% ethyl acetate hexanes and then 15% MeOH/ethyl acetate/0.2% HOAc to give the desired compound. To a solution of this olefin mixture (12 mg, 0.05 mmol) in MeOH (5 mL) was added c.HCl (2 drops) followed by Pd/C. The resulting mixture was stirred under a H₂ balloon for 16 hours. The reaction mixture was filtered through celite, the filtrate concentrated in vacuo and purified by reverse phase HPLC (Gilson) to give the desired compound.

LCMS m/z 219 (M+H).

### Examples 21-31

The following compounds were prepared under conditions similar to those described in EXAMPLE 20 above and illustrated in Scheme 4. The order of reaction steps may be switched regarding the olefin reduction and t-butyl ester cleavage. The TBAF deprotection of the N-toluenesulfonamide protecting group may also be conducted with aqueous LiOH, and the hydrogenation step may be catalyzed by either Pd—C or palladium hydroxide (Pearlman's catalyst). Pyrimidine EXAMPLE 28 was synthesized in the absence of the N-toluenesulfonamide protecting group.

| | | MS |
|---|---|---|
| | | m/z |
| EXAMPLE | STRUCTURE | (M + H) |
| 21 | 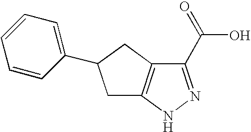 | 229 |
| 22 | 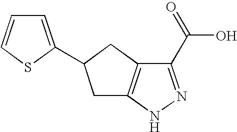 | 235 |
| 23 | 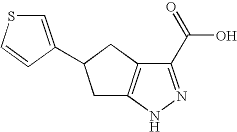 | 235 |
| 24 | 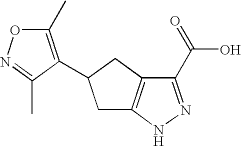 | 248 |
| 25 | 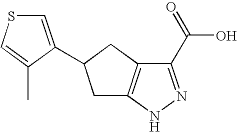 | 249 |
| 26 | 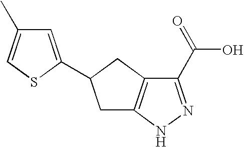 | 249 |
| 27 | 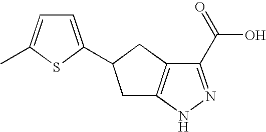 | 249 |
| 28 | 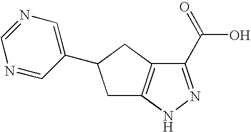 | 231 |
| 29 | 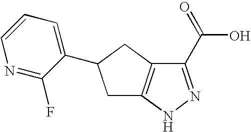 | 248 |
| 30 | 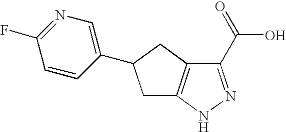 | 248 |
| 31 | 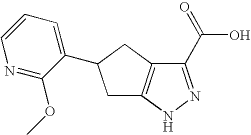 | 260 |

¹H NMR for selected examples:

### Example 21

### (500 MHz, DMSO-d₆) δ 2.90 (m, 2H), 3.10 (m, 2H), 4.10 (m, 1H), 7.20-7.30 (m, 5H).

### Example 25

### (500 MHz, CD₃OD) δ 2.20 (s, 3H), 2.90 (m, 2H), 3.20 (m, 2H), 4.15 (m, 1H), 6.95 (s, 1H), 7.15 (s, 1H).

### Example 26

### (500 MHz, DMSO-d₆) δ2.15 (s, 3H), 2.85 (m, 2H), 3.20 (m, 2H), 4.20 (m, 1H), 6.80 (s, 1H), 6.95 (s, 1H).

### Example 27

### (500 MHz, DMSO-d₆) δ 2.40 (s, 3H), 2.70 (m, 2H), 3.20 (m, 2H), 4.10 (m, 1H), 6.60 (s, 1H), 6.70 (s, 1H).

### Example 28

### (500 MHz, CD₃OD) δ 2.90 (m, 2H), 3.40 (m, 2H), 4.10 (m, 1H), 8.80 (s, 2H), 9.05 (s, 1H).

### Example 29

### (500 MHz, DMSO-d₆) δ 2.80 (m, 2H), 3.20 (m, 2H), 4.20 (m, 1H), 7.30 (t, 1H), 7.95 (t, 1H), 8.10 (d, 1H).

### Example 30

### (500 MHz, DMSO-d₆) δ 2.80 (m, 2H), 3.20 (m, 2H), 4.10 (m, 1H), 7.10 (dd, 1H), 7.98 (t, 1H), 8.20 (s, 1H).

### Example 31

### (500 MHz, DMSO-d₆) δ 2.75 (m, 2H), 3.05 (m, 2H), 3.91 (s, 3H), 4.10 (m, 1H), 6.90 (dd, 1H), 7.60 (d, 1H), 8.05 (d, 1H).

### Example 32

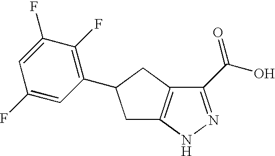

Commercially available cyclopentenone (1 mL, 12.3 mmol) was combined with 2,3,5-trifluorophenyl boronic acid (2.6 g, 14.8 mmol), sodium acetate (2 g, 24.6 mmol), palladium acetate (276 mg, 1.23 mmol), and SbCl₂ (280 mg, 1.23 mmol) in HOAc (123 mL). The reaction mixture was stirred for 18 h, concentrated in vacuo, diluted into methylene chloride, filtered over celite, and concentrated in vacuo. The crude product was purified by preparative centrifugal chromatography (SiO₂, 20% EtOAc-hexane) to afford the beta-substituted enone intermediate. This material (1.5 g, 4.8 mmol) was diluted into ethanol (100 mL), treated with potassium tert-butoxide (1M t-BuOH, 5.3 mL, 5.3 mmol) and diethyl oxalate (778 mg, 5.3 mmol) after which the colorless reaction mixture turns red. The reaction mixture was maintained for 1 h, monitored by LCMS, and then treated with hydrazine-hydrochloride (361 mg, 5.3 mmol). The reaction mixture was maintained for 15 h, concentrated in vacuo, diluted with (1:1) EtOAc-water, the organic phase separated, dried over anhydrous sodium sulfate, and concentrated in vacuo. The residue was purified by preparative centrifugal chromatography (SiO₂, 50% EtOAc-hexane) to afford the olefinic pyrazole ethyl ester. This olefin intermediate was reduced by treatment with ammonium formate (5 equivalents), formic acid (4 equivalents), and Pd—C (1 equivalent), in ethanol-water (7:1, 0.1 M). The reaction mixture was heated in a re-sealable pressure tube at 85° C. for 12 h, then cooled, filtered, concentrated in vacuo, partitioned between water and EtOAc, and the organic phase separated, dried over anhydrous sodium sulfate, and concentrated in vacuo. The crude ester (63 mg, 0.203 mmol) was diluted into water-THF (1:1, 2 mL) and treated with LiOH (14.5 mg, 0.609 mmol). The reaction mixture was maintained for 18 h, concentrated in vacuo to a small volume of water, acidified to pH 2, extracted with EtOAc, and the organic partition dried over anhydrous sodium sulfate and concentrated in vacuo. The residue was purified by preparative reverse phase HPLC on a Gilson system to afford the desired product.

¹H NMR (CD₃OD, 500 MHz) 2 apparent rotamers, (major) δ 7.03 (m, 1H), 6.97 (m, 1H), 4.37 (m, 1H), 3.25 (m, 2H), 2.89 (m, 2H) and (minor) δ 6.97 (m, 1H), 6.52 (m, 1H), 4.69 (dd, 1H), 3.25 (m, 2H), 2.89 (m, 2H); LCMS m/z 265 (M+H).

### Example 33

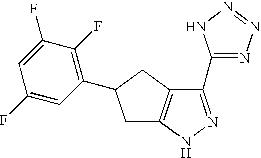

The ethyl ester intermediate from EXAMPLE 32 above (1.5 g, 4.87 mmol) was diluted into ammonium hydroxide (0.05 M) and dioxane (0.30 M), and the reaction mixture was stirred in a re-sealable pressure tube for 12 h. The mixture was concentrated in vacuo to afford the clean crude carboxamide intermediate. This carboxamide (400 mg, 1.51 mmol) was diluted into THF (10 mL), combined with triethylamine (0.5 mL, 3.5 mmol), the mixture cooled to −5° C., and then treated with trifluoroacetic anhydride (0.5 mL, 3.5 mmol). The reaction mixture was maintained at room temperature for 2 h, quenched with saturated aqueous sodium bicarbonate, concentrated, partitioned between water and methylene chloride, and the organic partition dried over anhydrous sodium sulfate and concentrated in vacuo. This nitrile intermediate (67 mg, 0.25 mmol) was combined with sodium azide (33 mg, 0.51 mmol) and ZnBr₂ (57 mg, 0.25 mmol), and diluted with (10:1) isopropanol-water (1.1 mL). The reaction mixture was heated at 85° C. in a re-sealable pressure tube for 24 h, cooled, concentrated, filtered, and purified by preparative reverse phase HPLC on a Gilson system to afford the desired product.

¹H NMR (CD₃OD, 500 MHz) 2 apparent rotamers, (major) δ 7.04 (m, 1H), 6.96 (m, 1H), 4.53 (m, 1H), 3.37 (m, 2H), 2.99 (m, 2H) and (minor) δ 6.96 (m, 1H), 6.45 (m, 1H), 4.83 (m, 1H), 3.27 (m, 2H), 2.92 (m, 2H); LCMS m/z 307 (M+H).

### Example 34

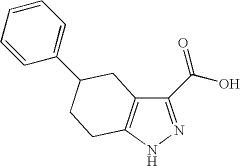

To a solution of Intermediate C (100 mg, 0.232 mmol) in DME (1.5 mL) was added phenyl boronic acid (34 mg, 0.279 mmol), triethyl amine (97 μL, 0.70 mmol) and tetrakis triphenyl phosphine palladium (0) (13 mg, 0.012 mmol). The resulting mixture was heated in the microwave for 10 minutes at 100° C. The reaction mixture was concentrated in vacuo and purified by Prep TLC plate using 40% ethyl acetate-hexanes as eluant to give the desired compound as yellow oil. To a solution of this ethyl ester (62 mg, 0.172 mmol) in dioxane (3 mL) was added 1N NaOH (1.74 mL). After stirring the reaction overnight at room temperature it was concentrated in vacuo to remove the dioxane. The residue was acidified with 1N HCl. The resulting mixture was extracted with ethyl acetate, washed with brine and dried over anhydrous Na₂SO₄. The solution was filtered and concentrated in vacuo to give a solid. To a solution of this olefin (34 mg, 0.102 mmol) in methanol (1.5 mL) was added c. HCl (300 μL) and Pd/C. The resulting mixture was stirred under a hydrogen balloon for 4 hours. The catalyst was filtered through celite. The filtrate was concentrated in vacuo and purified by reverse phase HPLC (Gilson) to give the desired compound as a yellow solid. LCMS m/z 243 (M+H).

### Example 35

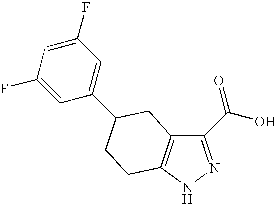

To a solution of Intermediate D (75 mg, 0.152 mmol) in DME (1.5 mL) was added 3,5-difluoro-phenyl boronic acid (29 mg, 0.182 mmol), triethyl amine (64 μL, 0.46 mmol) and tetrakis triphenyl phosphine palladium (0) (9 mg, 0.008 mmol). The resulting mixture was heated in the microwave for 10 minutes at 100° C. The reaction mixture was concentrated in vacuo and purified by Prep TLC plate using 20% ethyl acetate-hexanes as eluent to give the desired product. To a solution of this olefin (55 mg, 0.12 mmol) in 2:1 THF/MeOH (3 mL) was added ammonium formate (87 mg, 1.2 mmol), formic acid (300 μL) and Pd/C and the reaction was heated in a pressure tube at 80° C. for 48 hours. The reaction mixture was filtered through celite. The filtrate was concentrated in vacuo and re-dissolved in ethyl acetate. The organic layer was washed with water, saturated NaHCO₃ solution and brine. The organic layer was dried over anhydrous Na₂SO₄, filtered and concentrated in vacuo. This material was used in the next step without any further purification. To a solution of this ethyl ester in 1:1 THF/MeOH (2 mL) was added 1N LiOH (2 mL). The resulting solution was stirred at room temperature for 18 hours. The reaction mixture was concentrated in vacuo to remove the organic solvents. The residue was acidified with 1N HCl and extracted with ethyl acetate. The organic layer was washed with brine, dried over anhydrous Na₂SO₄, filtered and concentrated in vacuo. The residue was purified by reverse phase HPLC (Gilson) to give the desired compound.

¹H NMR (500 MHz, CD₃OD) δ 1.95 (m, 1H), 2.1 (bd, 1H), 2.67 (dd, 1H), 2.90 (m, 2H), 3.10 (m, 1H), 3.30 (dd, 1H), 6.78 (t, 1H), 6.93 (d, 2H); LCMS m/z 279 (M+H).

### Examples 36-44

The following compounds were prepared under conditions similar to those described in EXAMPLES 34 and 35 above and illustrated in Scheme 6.

| | | MS |
|---|---|---|
| | | m/z |
| EXAMPLE | STRUCTURE | (M + H) |
| 36 | 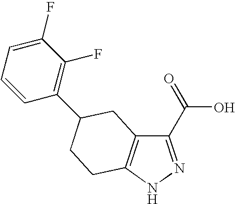 | 279 |
| 37 | 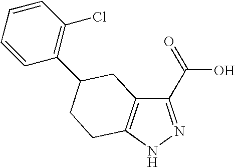 | 277 |
| 38 | 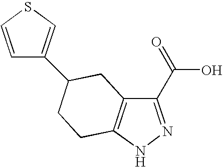 | 249 |
| 39 | 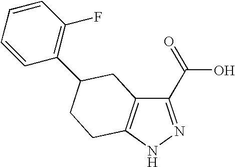 | 261 |
| 40 | 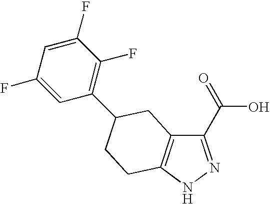 | 297 |
| 41 | 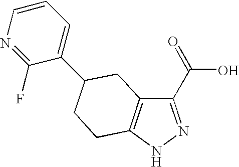 | 262 |
| 42 | 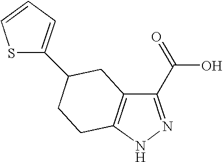 | 249 |
| 43 | 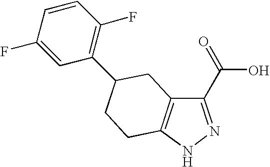 | 279 |
| 44 | 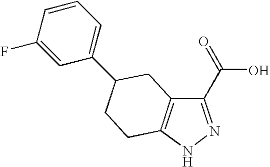 | 261 |

¹H NMR for selected examples:

### Example 36

### (500 MHz, CD₃OD) δ 2.10 (m, 2H), 2.80 (m, 3H), 3.16 (dd, 1H), 3.20 (m, 1H), 7.10 (m, 3H).

### Example 37

### (500 MHz, CD₃OD) δ 2.05 (m, 2H), 2.60 (d, 1H) 2.90 (m, 2H), 3.20 (dd, 1H), 3.46 (bm, 1H), 7.19 (t, 1H), 7.28 (t, 1H), 7.40 (t, 2H).

### Example 39

### (500 MHz, CD₃OD) δ 2.07 (m, 2H), 2.78 (m, 3H), 3.10 (dd, 1H), 3.25 (m, 1H), 7.04 (t, 1H), 7.14 (t, 1H), 7.23 (q, 1H), 7.32 (t, 1H).

### Example 40

### (500 MHz, CD₃OD) δ 2.05 (m, 2H), 2.90 (m, 3H), 3.20 (d, 2H), 7.00 (dd, 2H).

### Example 41

### (500 MHz, CD₃OD) δ 8.06 (d, 1H), 7.89 (t, 1H), 7.28 (t, 1H), 3.19 (m, 2H), 2.78 (m, 3H), 2.09 (m, 2H).

### Example 42

### (500 MHz, CD₃OD) δ 7.20 (d, 1H), 6.92 (m, 2H), 3.25 (m, 1H), 2.80 (m, 4H), 2.28 (m, 1H), 1.96 (m, 1H).

### Example 43

### (500 MHz, CD₃OD) δ 2.00 (m, 2H), 2.68 (dd, 1H), 2.80 (m, 2H), 3.10 (dd, 1H), 3.20 (m, 1H), 6.95 (m, 1H), 7.10 (m, 2H).

### Example 44

### (500 MHz, DMSO-d₆) δ 2.07 (m, 2H), 2.72-2.84 (m 3H), 3.12 (m, 1H), 3.27 (m, 1H), 7.00 (t, 1H), 7.16 (d, 2H), 7.35 (q, 1H).

### Example 45

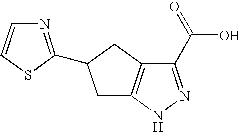

To a stirred solution of nBuLi (3.44 mL, 1.6 M, 5.5 mmol) in anhydrous Et₂O (20 mL) at −78° C. was added drop-wise over 5 minutes a solution of thiazole (425 mg, 5 mmol) in Et₂O (12.5 μL). After stirring the reaction mixture at −78° C. for 1 hour, a solution of 3-ethoxy cyclopentenone (630 mg, 5 mmol) in Et₂O was added drop-wise over 15 minutes. The reaction was warmed to room temperature slowly, and quenched with 1:1 MeOH/2N HCl. The resulting mixture was extracted with ethyl acetate. The organic layer was washed with brine, dried over anhydrous Na₂SO₄, filtered and concentrated in vacuo. The residue was purified by flash chromatography (Biotage flash 40M) using 40% ethyl acetate-hexanes as eluent to give the desired compound as a white solid (400 mg). To a solution of this cyclopentenone (400 mg, 2.42 mmol) in anhydrous THF (20 mL) cooled to −78° C. under a N₂ atmosphere was added LDA (1.27 mL, 2.0 M). After 15 minutes di-tert-butyl-dioxalate (0.538 g, 2.66 mmol) was added. The reaction was slowly warmed to room temperature, stirred for 1 hour, and quenched with 1N HCl. The resulting mixture was extracted with ethyl acetate, washed with brine, dried over anhydrous Na₂SO₄, filtered and concentrated in vacuo. The residue was purified by flash chromatography (Biotage 40M) using 30% ethyl acetate-hexanes as the eluant to give the desired compound. To a solution of this diketone (250 mg, 0.86 mmol) in ethanol (10 mL), was added hydrazine (29 μl) and glacial acetic acid (1 mL). After stirring at room temperature overnight, the reaction mixture was concentrated in vacuo and purified by flash chromatography (Biotage 40M) to give the desired compound. To a solution of this olefin (55 mg, 0.19 mmol) in EtOAc (9 mL) was added EtOH (5 mL), followed by catalytic amount of Pt₂O. The reaction was stirred under a hydrogen balloon for 48 hours. The reaction was filtered through celite and purified by flash chromatography (Biotage 20M) using 40% ethyl acetate-hexanes. This material was then treated with 50% TFA/DCM (2 mL) for 2 hours. The reaction mixture was concentrated in vacuo to give the desired compound.

¹H NMR (500 MHz, CD₃OD) δ 3.05 (m, 2H), 3.40 (m, 2H), 4.50 (m, 1H), 7.50 (s, 1H), 7.80 (s, 1H); LCMS m/z 236 (M+H).

### Examples 46-50

The following compounds were prepared under conditions similar to those described in EXAMPLE 45 above and illustrated in Scheme 7. Cyclohexyl EXAMPLE 46 was prepared via cyclohexyl magnesium chloride Grignard addition to 3-ethoxy-cyclopentenone. This 3-(cyclohexyl)-cyclopentenone was carried through similar transformations shown in Scheme 7, including diethyl oxalate acylation, pyrazole formation, LiOH saponification of the ethyl ester, and final hydrogenation of the olefin. EXAMPLES 47 and 49 were synthesized via lithium-halide exchange of 2-bromopyridine with n-butyllithium, and 2-chloropyrazine with lithium tetramethylpiperidinyl amide, respectively. The 3-(pyrazinyl)-cyclopentenone intermediate of EXAMPLE 49 was carried through similar transformations shown in Scheme 7, including diethyl oxalate acylation, pyrazole formation, hydrogenation, and LiOH saponification of the final ethyl ester. For the alpha-lithiation of N-methylpyrazole to generate EXAMPLE 48, see Tetrahedron 1983, 39(12), 2023. EXAMPLE 50 was synthesized via meta-bromopyridine exchange with isopropyl magnesium chloride to generate the 3-pyridyl Grignard reagent.

| | | MS |
|---|---|---|
| | | m/z |
| EXAMPLE | STRUCTURE | (M + H) |
| 46 | 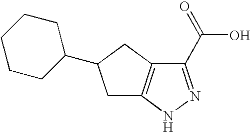 | 235 |
| 47 | 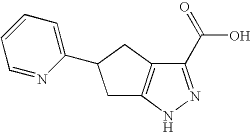 | 230 |
| 48 | 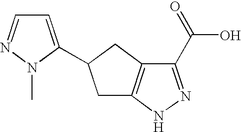 | 233 |
| 49 | 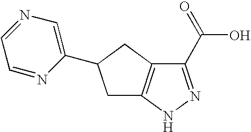 | 231 |
| 50 | 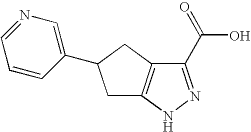 | 230 |

¹H NMR for selected examples:

### Example 47

### (500 MHz, DMSO-d₆) δ 2.90 (m, 2H), 3.00 (m, 2H), 4.10 (m, 1H), 7.20 (dd, 1H), 7.35 (d, 1H), 7.70 (t, 1H), 8.50 (d, 1H).

### Example 48

(500 MHz, D₂O) major rotamer, δ 7.94 (s, 1H), 6.55 (s, 1H), 4.39 (m, 1H), 4.05 (s, 3H), 3.37 (m, 2H), 2.94 (m, 2H).

### Example 49

### (500 MHz, CD₃OD) δ 3.05 (m, 2H), 3.20 (m, 1H), 3.40 (m, 1H), 4.35 (m, 1H), 8.45 (d, 1H), 8.57 (t, 1H), 8.61 (d, 1H).

### Example 50

### (500 MHz, CD₃OD) δ 8.55 (s, 1H), 8.40 (d, 1H), 7.81 (d, 1H), 7.40 (m, 1H), 4.10 (m, 1H), 3.35 (dd, 1H), 3.20 (dd, 1H), 2.90 (dd, 1H), 2.80 (dd, 1H).

### Example 51

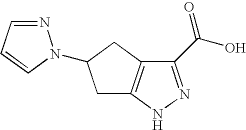

Commercially available cyclopentenone (2 mL, 24.7 mmol) was combined with pyrazole (3.3 g, 49.4 mmol) in chloroform (10 mL), and added to a re-sealable pressure tube. The reaction mixture was warmed at 50° C. for 24 h, cooled, concentrated, and purified by preparative centrifugal chromatography (SiO₂, EtOAc-hexane) to afford the beta-substituted cyclopentanone intermediate. In a similar manner to EXAMPLE 32 as shown in Scheme 5, this material was treated with potassium tert-butoxide and diethyl oxalate in ethanol, followed by hydrazine-hydrochloride. The ethyl ester intermediate was also saponified in a similar manner except that NaOH was used in place of LiOH, and the residue was purified by preparative reverse phase HPLC on a Gilson system to afford the desired product.

¹H NMR (CD₃OD, 500 MHz) δ 8.14 (m, 1H), 8.02 (m, 1H), 6.61 (m, 1H), 5.85 (m, 1H), 3.59 (m, 2H), 3.28 (m, 2H); LCMS m/z 219 (M+H).

### Example 52

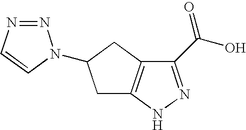

EXAMPLE 52 was prepared from 1,2,3-triazole under conditions similar to those described in EXAMPLE 51 above and illustrated in Scheme 7.

¹H NMR (500 MHz, CD₃OD) δ 3.20 (m, 2H), 3.50 (m, 2H), 5.90 (m, 1H), 7.72 (d, 2H); LCMS m/z 220 (M+H).

### Example 53

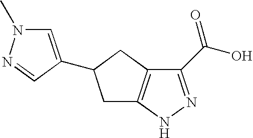

3-Iodo-cyclopentenone (50 mg, 0.16 mmol), described in the literature (Can. J. Chem. 1982, 60, 210), was combined with the commercially available boronate ester (65 mg, 0.31 mmol) shown in Scheme 7, and potassium phosphate (166 mg, 0.78 mmol) in DME (10 mL). The reaction mixture was treated with Pd(dppf)Cl₂ (0.031 mmol) and heated at 100° C. for 14 h. The reaction mixture was then cooled, concentrated, and purified by preparative centrifugal chromatography (SiO₂, EtOAc-hexane) to afford the beta-substituted cyclopentenone intermediate. In a similar manner to EXAMPLE 32 as shown in Scheme 5, this material was treated with potassium tert-butoxide and diethyl oxalate in ethanol, followed by hydrazine-hydrochloride to afford the olefinic pyrazole ethyl ester. This olefin ester intermediate was reduced by treatment with ammonium formate, formic acid, Pd—C, and saponified in a similar manner with aqueous NaOH. The residue was purified by preparative reverse phase HPLC on a Gilson system to afford the desired product.

¹H NMR (CD₃OD, 500 MHz) δ 7.59 (s, 1H), 7.51 (s, 1H), 4.03 (m, 1H), 3.88 (s, 3H), 3.25 (dd, 1H), 3.17 (dd, 1H), 2.77 (m, 2H); LCMS m/z 233 (M+H).

### Example 54

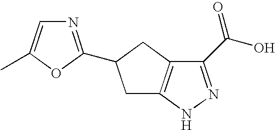

Commercially available 3-carboxy-cyclopentanone (100 mg, 0.78 mmol) was diluted into benzene (20 mL), treated with thionyl chloride (0.093 mL, 0.78 mmol), and the reaction mixture heated at reflux for 2 h. The mixture was then concentrated, diluted with methylene chloride (20 mL), cooled to 0° C., and propargyl amine added (78 mg, 0.86 mmol), followed by triethylamine (0.33 mL, 2.3 mmol). The reaction mixture was aged for 12 h, then partitioned with saturated aqueous sodium bicarbonate, the organic partition dried over anhydrous sodium sulfate and concentrated in vacuo. The residue was purified by preparative centrifugal chromatography (SiO₂, EtOAc-hexane) to afford the beta-amide intermediate. This propargyl amide (594 mg, 3.6 mmol) was diluted into HOAc (10 mL), treated with Hg(OAc)₂ (37 mg, 0.12 mmol), and heated at reflux for 2 h. The reaction mixture was then cooled, filtered, concentrated, and partitioned between saturated aqueous sodium bicarbonate and methylene chloride. The organic partition was dried over anhydrous sodium sulfate, concentrated in vacuo, and the residue was purified by preparative centrifugal chromatography (SiO₂, EtOAc-hexane) to afford the beta-oxazole intermediate (90 mg). In a similar manner to EXAMPLE 32 as shown in Scheme 5, this material was treated with potassium tert-butoxide and diethyl oxalate in ethanol, followed by hydrazine-hydrochloride to afford the pyrazole ethyl ester. This ester intermediate was saponified in a similar manner with aqueous NaOH. The residue was purified by preparative reverse phase HPLC on a Gilson system to afford the desired product.

¹H NMR (CD₃OD, 500 MHz) δ 6.71 (s, 1H), 4.26 (m, 1H), 3.25 (m, 2H), 3.10 (dd, 2H), 2.32 (s, 3H); LCMS m/z 234 (M+H).

### Example 55

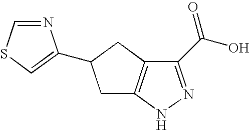

To a solution of hexabutyl-distannane (5.8 g, 10 mmol) in anhydrous THF cooled to −78° C. under a nitrogen atmosphere was added nBuLi (6.25 mL, 1.6 M, 10 mmol). After 1 hour, a solution of 3-ethoxy-cyclopentenone (1.26 g, 10 mmol) in THF (5 mL) was added dropwise. The reaction was stirred at −78° C. for 1 hour and then quenched by pouring into a solution of saturated ammonium chloride. The resulting mixture was extracted with ethyl acetate, washed with brine and dried over anhydrous Na₂SO₄. The organic layer was filtered and concentrated in vacuo give a yellow oil. This material was purified by flash chromatography (SiO₂) using 10% ethyl acetate hexanes to give the desired stannane as a colorless oil.

To a solution of 2,4-dibromo thiazole (1.0 g, 4.11 mmol) in anhydrous ether (50 mL) cooled to −78° C. under a nitrogen atmosphere was added nBuLi (3.08 mL, 4.94 mmol, 1.6 M solution). After stirring at −78° C. for 1 hour chlorotrimethyl silane (0.57 mL, 4.52 mmol) was added. The reaction was stirred at −78° C. for an additional hour and quenched by adding saturated NaHCO₃ solution. The resulting mixture was extracted with ethyl acetate. The organic layer was washed with brine, dried over anhydrous Na₂SO₄, filtered and concentrated in vacuo. The residue was purified by flash chromatography (SiO₂) using 10% ethyl acetate-hexanes to give the desired TMS-bromothiazole as a colorless oil.

The union of the two stannane and bromide intermediates, as shown in Scheme 9, was conducted as follows. To a solution of the cyclopentenone stannane (390 mg, 1.05 mmol) and the TMS-bromothiazole (206 mg, 0.87 mmol) in toluene (5 mL), was added dichloro palladium (bis triphenyl phosphine). The resulting mixture was heated to reflux for 18 hours. The reaction was cooled to room temperature. A saturated solution of KF (5 mL) was added and the resulting mixture was stirred vigorously for 1 hour. The reaction mixture was filtered through celite. The filtrate was washed with water, brine, dried over anhydrous Na₂SO₄, filtered and concentrated in vacuo. The residue was purified by flash chromatography (SiO₂) using 40% ethyl acetate-hexanes to give the desired product. To a solution of this beta-substituted cyclopentenone (67 mg, 0.28 mmol) in anhydrous ethanol (5 mL) was added diethyl oxalate (38 μL, 0.28 mmol) and potassium-t-butoxide (282 μL, 0.28 mmol, 1.0 M in THF). After stirring at room temperature for 2 hours, a solution of hydrazine hydrochloride (23 mg, 0.338 mmol) in water (0.5 mL) was added. The reaction was stirred at room temperature for 16 hours and then heated to reflux for 15 minutes. The reaction mixture was concentrated in vacuo. The residue was diluted with ethyl acetate, washed with water, saturated NaHCO₃ solution, filtered and concentrated in vacuo to give the desired product as a single double bond isomer. To a solution of this olefin (12 mg, 0.045 mmol) in EtOAc/EtOH (2.8:1, 2 mL) was added Pd(OH)₂ (5 mg) and the reaction stirred under a hydrogen balloon for 4 hours. The reaction mixture was filtered through celite. The filtrate was concentrated in vacuo and purified by flash chromatography (SiO₂) using 60% ethyl acetate-hexanes to give the desired product. To a solution of this ethyl ester in 1:1 THF/MeOH (2 mL), was added 1N NaOH (0.5 mL). After stirring the reaction at room temperature for 16 hours, it was acidified by the addition of 1N HCl (0.5 mL). The resulting mixture was purified by reverse phase HPLC to give the desired product.

¹H NMR (500 MHz, CD₃OD) [symbol] 3.00 (m, 2H), 3.30 (m, 2H), 4.40 (m, 1H), 7.40 (s, 1H), 9.10 (s, 1H); LCMS m/z 236 (M+H).

### DP Example 1

### [5-[(4-Chlorophenyl)thio]-4-(methylsulfonyl)-6,7,8,9-tetrahydropyrido[3,2-b]indolizin-6-yl]acetic acid (Compound G)

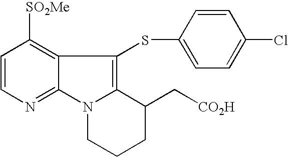

### Step 1 4-Chloronicotinaldehyde

The title compound was prepared as described by F. Marsais et al., J. Heterocyclic Chem., 25, 81 (1988).

### Step 2 4-(Methylthio)micotinaldehyde

To a solution of NaSMe (9.5 g, 135 mmol) in MeOH (250 mL) was added the 4-chloronicotinaldehyde (13.5 g, 94.4 mmol) of Step 1 in MeOH (250 mL). The reaction mixture was maintained at 60° C. for 15 min. The reaction mixture was poured over NH₄Cl and EtOAc. The organic phase was separated, washed with H₂O and dried over Na₂SO₄. The compound was then purified over silica gel with 50% EtOAc in Hexanes to provide the title compound.

### Step 3 Methyl (2Z)-2-azido-3-[4-(methylthio)pyridin-3-yl]prop-2-enoate

A solution of 4-(methylthio)nicotinealdehyde (4.8 g, 31 mmol) and methyl azidoacetate (9.0 g, 78 mmol) in MeOH (50 mL) was added to a solution of 25% NaOMe in MeOH (16.9 mL, 78 mmol) at −12° C. The internal temperature was monitored and maintained at −10° C. to −12° C. during the min. addition. The resulting mixture was then stirred in an ice bath for several hours, followed by overnight in an ice bath in the cold room. The suspension was then poured onto a mixture of ice and NH₄Cl, and the slurry was filtered after 10 min. of stirring. The product was washed with cold H₂O and was then dried under vacuum to give the title compound as a beige solid, which contained some salts. The compound is then purified over silica gel with EtOAc.

### Step 4 Methyl 4-(methylthio)-1H-pyrrolo[2,3-b]pyridine-2-carboxylate

A suspension of the compound of Step 3 (0.40 g, 1.6 mmol) in xylenes (16 mL) was heated slowly to 140° C. After a period of 15 min. at 140° C., the yellow solution was cooled to room temperature. Precaution must be taken due to the possibility of an exotherme due to the formation of nitrogen. The suspension was then cooled to 0° C., filtered and washed with xylene to provide the title compound.

### Step 5 Ethyl 4-(methylthio)-6-oxo-6,7,8,9-tetrahydropyrido[3,2-b]indolizine-7-carboxylate

To a solution of the compound of Step 4 (0.35 g, 1.6 mmol) in DMF (20 mL) at 0° C. was added NaH (1.2 eq.). After a period of 5 min., nBu₄NI (0.10 g) and ethyl 4-bromobutyrate (0.40 mL). were added. After a period of 1 h at room temperature, the reaction mixture was poured over saturated NH₄Cl and EtOAc. The organic phase was separated, washed with H₂O and dried over NaSO₄. After evaporation the crude product was purified by flash chromatography. The bis ester was then dissolved in THF (7.0 mL) and a 1.06 M of THF solution of potassium tert-butoxide (2.2 mL) was added at 0° C. After a period of 1 h at room temperature, the reaction mixture was then poured over saturated NH₄Cl and EtOAc. The organic phase was separated, dried over Na₂SO₄ and evaporated under reduced pressure to provide the title compound as a mixture of ethyl and methyl ester.

### Step 6 4-(Methylthio)-8,9-dihydropyrido[3,2-b]indolizin-6(7H)-one

To the compound of Step 5, (0.32 g) were added EtOH (8.0 mL) and concentrated HCl (2.0 mL). The resulting suspension was refluxed for 5 h. The reaction mixture was partitioned between EtOAc and Na₂CO₃. The organic phase was separated and evaporated to provide the title compound.

### Step 7 Ethyl (2E,2Z)-[4-(methylthio)-8,9-dihydropyrido[3,2-b]indolizin-6(7H)-ylidene]ethanoate

To a DMF solution (12 mL) of triethyl phosphonoacetate (0.45 g, 2.17 mmol) were added 80% NaH (0.06 g, 2.00 mmol) and the compound of Step 6 (0.22 g, 1.00 mmole). After a period of 4 h at 55° C., the reaction mixture was poured over saturated NH₄Cl and EtOAc. The organic phase was separated and evaporated under reduced pressure. The crude product was purified by flash chromatography to afford the title compound.

### Step 8 Ethyl[4-(methylthio)-6,7,8,9-tetrahydropyrido[3,2-b]indolizin-6-yl]acetate

The compound of Step 7 was dissolved in MeOH-THF using heat for dissolution. To the previous cooled solution was added at room temperature PtO₂ and the resulting mixture was maintained for 18 h under an atmospheric pressure of hydrogen. The reaction mixture was filtered carefully over Celite using CH₂Cl₂. The filtrate was evaporated under reduced pressure to provide the title compound. Alternatively, the compound of Step 7 can be hydrogenated with Pd (OH)₂ in EtOAc at 40 PSI of H₂ for 18 h.

### Step 9 Ethyl[4-(methylsulfonyl)-6,7,8,9-tetrahydropyrido[3,2-b]indolizin-6-yl]acetate

To the compound of Step 8 (0.08 g, 0.27 mmol) in MeOH (3.0 mL) were added Na₂WO4 (0.10 g) and 30% H₂O₂ (600 μL). After a period of 1 h, the reaction mixture was partitioned between H₂O and EtOAc. The organic phase was washed with H₂O, separated and evaporated. The title compound was purified by flash chromatography.

### Step 10 Ethyl[5-[(4-chlorophenyl)thio]-4-(methylsulfonyl)-6,7,8,9-tetrahydropyrido[3,2-b]indolizin-6-yl]acetate

To a 1,2-dichloroethane solution (2.0 mL) of 4,4′-dichlorodiphenyl disulfide (0.24 g) was added SO₂Cl₂ (50 μL). To the compound of Step 9 (0.05 g) in DMF (2.0 mL) was added the previous mixture (≈180 μL). The reaction was followed by ¹H NMR and maintained at room temperature until no starting material remained. The reaction mixture was poured over saturated NaHCO₃ and EtOAc. The organic phase was separated, evaporated and the title compound purified by flash chromatography.

### Step 11 [5-[(4-Chlorophenyl)thio]-4-(methylsulfonyl)-6,7,8,9-tetrahydropyrido[3,2-b]indolizin-6-yl]acetic acid

To the compound of Step 10 dissolved in a 1/1 mixture of THF-MeOH was added 1N NaOH. After a period of 18 h at room temperature, the reaction mixture was partitioned between saturated NH₄Cl and EtOAc. The organic phase was separated, dried over Na₂SO₄ and evaporated to provide the title compound.

¹H NMR (500 MHz, acetone-d₆) δ 11.00 (bs, 1H), 8.60 (d, 1H), 7.80 (d, 1H), 7.20 (d, 2H), 7.00 (d, 2H), 4.65 (m, 1H), 4.20 (m, 1H), 3.75 (m, 1H), 3.35 (s, 3H), 2.80 to 2.10 (m, 6H).

### DP Example 2

### [5-[(4-Chlorophenyl)thio]-4-(methylthio)-6,7,8,9-tetrahydropyrido[3,2-b]indolizin-6-yl]acetic acid (Compound H)

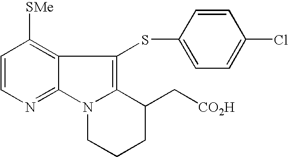

The title compound can be prepared from the compound of Example 1, Step 8 in a similar manner as described in Example 1, Step 10 and 11.

m/z 418.

### DP Example 3

### [5-[(3,4-Dichlorophenyl)thio]-4-(methylsulfonyl)-6,7,8,9-tetrahydropyrido[3,2-b]indolizin-6-yl]acetic acid (Compound I)

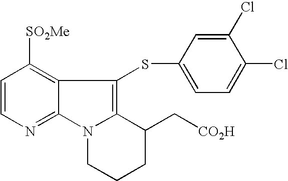

The title compound was prepared as described in Example 1 using bis(3,4-dichlorophenyl)disulfide in Step 10.

¹H NMR (500 MHz, acetone-d₆) δ 8.55 (d, 1H), 7.85 (d, 1H), 7.35 (d, 1H), 7.15 (s, 1H), 6.95 (d, 1H), 4.60 (m, 1H), 4.15 (m, 1H), 3.80 (m, 1H), 3.40 (s, 3H), 2.80 to 2.10 (m, 6H). m/z 484.

The enantiomers were separated on a Chiralecel OD column 25 cm×20 mm using 30% isopropanol 17% ethanol 0.2% acetic acid in hexane, flow rate 8 ml/min. Their purities were verified on a Chiralecel OD column 25 cm×4.6 mm using 35% isopropanol 0.2% acetic acid in hexane, flow rate 1.0 ml/min. More mobile enantiomer Tr=9.7 min, less mobile enantiomer Tr 11.1 min.

### DP Example 4

### [5-(4-Chlorobenzoyl)-4-(methylsulfonyl-6,7,8,9-tetrahydropyrido[3,2-b]indolizin-6-yl]acetic acid (Compound J)

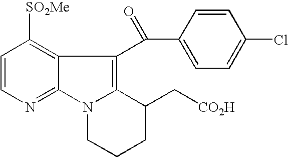

### Step 1 Ethyl[5-(4-chlorobenzoyl)-4-(methylthio)-6,7,8,9-tetrahydropyrido[3,2-b]indolizin-6-yl]acetate

To a solution of 4-chlorobenzoyl chloride (0.30 g, 1.7 mmol) in 1,2-dichloethane (6.0 mL) was added AlCl₃ (0.24 g, 1.8 mmole). After a period of 5 min. a solution of ethyl[4-(methylthio)-6,7,8,9-tetrahydropyrido[3,2-b]indolizin-6-yl]acetate from Example 1 Step 8 (0.15 g, 0.47 mmole) in 1,2-dichloroethane (6.0 mL) was added to the previous mixture. After a period of 4 h, at 80° C., the reaction mixture was partitioned between EtOAc and NaHCO₃. The organic phase was separated, dried over Na₂SO₄ and evaporated. The title compound was purified by flash chromatography.

### Step 2 Ethyl[5-(4-chlorobenzoyl)-4-(methylsulfonyl)-6,7,8,9-tetrahydropyrido[3,2-b]indolizin-6-yl]acetate

To a solution of ethyl[5-(4-chlorobenzoyl)-4-(methylthio)-6,7,8-9-tetrahydropyrido[3,2-b]indolizin-6-yl]acetate (0.12 g, 0.27 mmole) in MeOH (5.0 mL) were added Na₂WO4 (0.1 g) and 30% H₂O₂ (300 μL). The reaction mixture was stirred at 55° C. for 1 h. The reaction mixture was then partitioned between H₂O and EtOAc. The organic phase was washed with H₂O, dried over Na₂SO₄ and evaporated. The title compound was purified by flash chromatography.

### Step 3 [5-(4-Chlorobenzoyl)-4-(methylsulfonyl)-6,7,8,9-tetrahydropyrido[3,2-b]indolizin-6-yl]acetic acid

Ethyl[5-(4-chlorobenzoyl)-4-(methylsulfonyl)-6,7-8,9-tetrahydropyrido[3,2-b]indolizin-6-yl]acetate was treated as described in Example 1 Step 11 to provide the title compound.

¹H NMR (500 MHz, acetone-d₆) δ 8.55 (d, 1H), 7.90 (d, 2H), 7.65 (d, 1H), 7.45 (d, 2H), 4.55 (m, 1H), 4.25 (m, 1H), 3.45 (m, 1H), 3.20 (s, 3H), 2.05 to 3.00 (m, 6H). m/z 446.

### DP Example 5

### [5-(4-Bromophenyl)thio]-4-(methylsulfonyl)-6,7,8,9-tetrahydropyrido[3,2-b]indolizin-6-yl]acetic acid (Compound K)

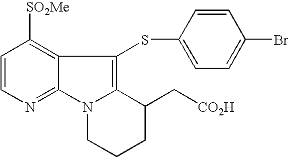

The title compound was prepared as described in Example 1 using 4,4′-dibromodiphenyl disulfide.

¹H NMR (500 MHz, Acetone-d₆) δ 8.60 (d, 1H), 7.80 (d, 1H), 7.35 (d, 2H), 7.00 (d, 2H), 4.65 (m, 1H), 4.20 (m, 1H), 3.80 (m, 1H), 3.35 (s, 3H), 2.80 to 2.10 (m, 6H).

### DP Example 6 Method-1

### [9-[(3,4-Dichlorophenyl)thio]-1-(methylsulfonyl)-7,8-dihydro-6H-pyrido[3,4-b]pyrrolizin-8-yl]acetic acid (Compound L)

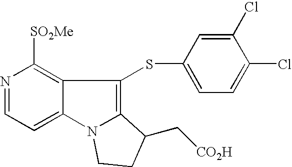

### Step 1 2-(Methylthio)micotinaldehyde

The title compound was prepared from 2-bromonicotinaldehyde (A. Numata Synthesis 1999 p. 306) as described in Example 1 Step 2 except the solution was heated at 55° C. for 2 hr.

### Step 2 Methyl (2Z)-2-azido-3-[2-(methylthio)pyridin-3-yl]prop-2-enoate

The title compound was prepared as described in Example 1 Step 3.

### Step 3 Methyl 4-(methylthio)-1H-pyrrolo[3,2-c]pyridine-2-carboxylate

A solution of methyl (2Z)-2-azido-3-[2-(methylthio)pyridin-3-yl]prop-2-enoate (1.00 g, 4.00 mmol) in mesitylene (50 mL) was heated at 160° C. for a period of 1 h. The reaction mixture was cooled to room temperature then to 0° C., the precipitate was filtered and washed with cold mesitylene to provide the title compound.

### Step 4 Methyl 1-(methylthio)-8-oxo-7,8-dihydro-6H-pyrido[3,4-b]pyrrolizine-7-carboxylate

To a suspension of methyl 4-(methylthio)-1H-pyrrolo[3,2-c]pyridine-2-carboxylate (0.30 g, 1.35 mmol) in THF (3 mL)—toluene (12.0 mL) were added a 1.06 M THF solution of potassium tert-butoxide (1.42 mL/1.41 mmol) and methyl acrylate (300 μL). The resulting mixture was heated at 80° C. for 18 h. The mixture was partitioned between EtOAc and NH₄Cl, and filtered through Celite. The organic phase was separated, dried over Na₂SO₄ and filtered, to provide the title compound.

### Step 5 1-(Methylthio)-6,7-dihydro-8H-pyrido[3,4-b]pyrrolizin-8-one

Methyl 1-(methylthio)-8-oxo-7,8-dihydro-6H-pyrido[3,4-b]pyrrolizine-7-carboxylate was converted to the title compound as described in Example 1 Step 6.

### Step 6 Methyl[8-hydroxy-1-(methylthio)-7,8-dihydro-6H-pyrido[3,4-b]pyrrolizin-8-yl]acetate

A mixture of 1-(methylthio)-6,7-dihydro-8H-pyrido[3,4-b]pyrrolizin-8-one (0.15 g, 0.68 mmol), methyl bromoacetate (0.34 mL), Zn—Cu (0.226 g) in THF (3.0 mL) was sonicated for 2 h. The mixture was then heated at 60° C. for 5 min. until completion of the reaction. The reaction mixture was partitioned between EtOAc and NH₄Cl. The organic phase was separated, dried over Na₂SO₄, filtered and evaporated under reduced pressure to provide the title compound. The compound was purified by flash chromatography.

### Step 7 Methyl[1-(methylthio)-7,8-dihydro-6H-pyrido[3,4-b]pyrrolizin-8-yl]acetate

To NaI (0.300 g) in CH₃CN (3.2 mL) was added TMSCl (0.266 mL). This mixture was added to a suspension of methyl[8-hydroxy-1-(methylthio)-7,8-dihydro-6H-pyrido[3,4-b]pyrrolizin-8-yl]acetate (0.15 g, 0.515 mmol) in CH₃CN (1.5 mL), in a water bath. After a period of 0.5 h, the reaction mixture was partitioned between EtOAc and NaHCO₃. The organic phase was separated, washed with sodium thiosulphate, dried over MgSO₄ and evaporated. The title compound was purified by flash chromatography.

### Step 8 Methyl[1-(methylsulfonyl)-7,8-dihydro-6H-pyrido[3,4-b]pyrrolizin-8-yl]acetate

Methyl[1-(methylthio)-7,8-dihydro-6H-pyrido[3,4-b]pyrrolizin-8-yl]acetate was converted to the title compound as described in Example 1 Step 9.

### Step 9 [9-[(3,4-Dichlorophenyl)thio]-1-(methylsulfonyl)-7,8-dihydro-6H-pyrido[3,4-b]pyrrolizin-8-yl]acetic acid

Methyl[1-(methylsulfonyl)-7,8-dihydro-6H-pyrido[3,4-b]pyrrolizin-8-yl]acetate was converted to the title compound as described in Example 1, Steps 10 and 11, using bis(3,4-dichlorophenyl)disulfide in Step 10.

¹H NMR (500 MHz, acetone-d₆) δ 8.35 (d, 1H) 7.80 (d, 1H), 7.35 (d, 1H), 7.15 (s, 1H), 6.95 (d, 1H), 4.55 (m, 1H), 4.35 (m, 1H), 3.90 (m, 1H), 3.30 (s, 3H), 3.15 (m, 1H), 3.05 (m, 1H), 2.80 (m, 1H), 2.50 (m, 1H).

### DP Example 6 Method-2

### [9-[(3,4-Dichlorophenyl)thio]-1-(methylsulfonyl)-7,8-dihydro-6H-pyrido[3,4-b]pyrrolizin-8-yl]acetic acid

### Step 1 1-(Methylthio)-7,8-dihydro-6H-pyrido[3,4-b]pyrrolizin-8-ol

To a suspension of 1-(methylthio)-6,7-dihydro-8H-pyrido[3,4-b]pyrrolizin-8-one from Example 6, Method-1 Step 5 (0.55 g, 2.2 mmol) in EtOH (10 mL)-THF (1 mL) was added NaBH₄ (0.10 g, 2.6 mmol) at 0° C. After a period of 30 min. at room temperature, the reaction was quenched by the addition of acetone. The solvents were evaporated under reduced pressure and EtOAC and H₂O were added to the residue. The organic phase was separated, dried over MgSO₄ and evaporated. The title compound was washed with EtOAc/Hexane and filtered.

### Step 2 Dimethyl 2-[1-(methylthio)-7,8-dihydro-6H-pyrido[3,4-b]pyrrolizin-8-yl]malonate

To a suspension of 1-(methylthio)-7,8-dihydro-6H-pyrido[3,4-b]pyrrolizin-8-ol (0.54 g, 2.1 mmol) in THF (10 mL) at −78° C. were added 1M NaHMDS in THF (2.35 mL, 2.4 mmol) and diphenyl chlorophosphate (0.53 mL, 2.6 mmol). After a period of 30 min. dimethyl malonate (0.73 mL, 6.4 mmol) and 1M NaHMDS in THF (6.8 mL, 6.8 mmol) were added. The reaction mixture was brought to 0° C. and then to room temperature. The mixture was then partitioned between ETOAc and NH₄Cl. The organic phase was dried over MgSO₄, filtered and evaporated. The title compound was purified by flash chromatography.

### Step 3 Methyl[1-(methylthio)-7,8-dihydro-6H-pyrido[3,4-b]pyrrolizin-8-yl]-acetate

To a mixture of dimethyl 2-[1-(methylthio)-7,8-dihydro-6H-pyrido[3,4-b]pyrrolizin-8-yl]malonate (0.59 g, 2.17 mmol) and DMSO (4 mL) was added NaCl (0.45 g) in H₂O (0.45 mL). After a period of 18 h at 150° C., the reaction mixture was partitioned between ETOAc and H₂O. The organic phase was separated, dried over Na₂SO₄ and evaporated. The title compound was then purified by flash chromatography.

### Step 4 [9-[(3,4-Dichlorophenyl)thio]-1-(methylsulfonyl)-7,8-dihydro-6H-pyrido[3,4-b]pyrrolizin-8-yl]acetic acid

The title compound was obtained from methyl[1-(methylthio)-7,8-dihydro-6H-pyrido[3,4-b]pyrrolizin-8-yl]acetate as described in Example 6, Method-1, Steps 8 to 9.

### DP Example 7

### [10-[(3,4-Dichlorophenyl)sulfanyl]-1-(methylsulfonyl)-6,7,8,9-tetrahydropyrido[3,4-b]indolizin-9-yl]acetic acid (Compound M)

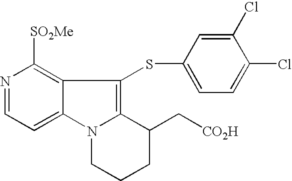

### Step 1 Ethyl[1-(methylsulfonyl)-6,7,8,9-tetrahydropyrido[3,4-b]indolizin-9-yl]acetate

The title compound was prepared from the product of Example 6, Step 3 in the same manner as described in Example 1, Steps 5 to 9.

### Step 2 [10-[(3,4-Dichlorophenyl)sulfanyl]-1-(methylsulfonyl)-6,7,8,9-tetrahydropyrido[3,4-b]indolizin-9-yl]acetic acid

The product of Step 1 was converted to the title compound in the same manner as Example 1, Steps 10-11, using bis(3,4-dichlorophenyl)disulfide in Step 10.

MS M+1=485.

### DP Example 8

### (4-(Methylsulfonyl)-5-{[4-(trifluoromethyl)phenyl]thio}-6,7,8,9-tetrahydropyrido[3,2-b]indolizin-6-yl)acetic acid (Compound N)

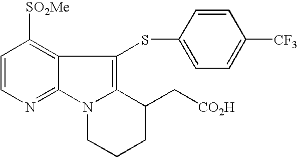

The title compound was prepared as described in Example 1 using bis[4-trifluoromethyl)phenyl]disulfide.

¹H NMR (500 MHz, acetone-d₆) δ 8.55 (d, 1H), 7.75 (d, 1H), 7.45 (d, 2H), 7.15 (d, 2H), 4.55 (m, 1H), 4.15 (m, 1H), 3.80 (m, 1H), 3.30 (s, 3H), 2.80 to 2.10 (m, 6H). m/z 513 (M+1).

### DP Example 9

### [5-[(2-Chloro-4-fluorophenyl)thio]-4-(methylsulfonyl)-6,7,8,9-tetrahydropyrido[3,2-b]indolizin-6-yl]acetic acid (Compound O)

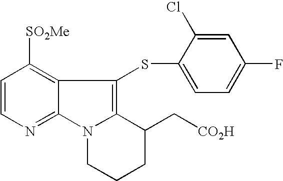

The title compound was prepared as described in Example 1 using bis(2-chloro-4-fluorophenyl)disulfide.

### m/z 469 (M+1).

### DP Example 10

### [4-(Methylsulfonyl)-5-(2-naphthylthio)-6,7,8,9-tetrahydropyrido[3,2-b]indolizin-6-yl]acetic acid (Compound P)

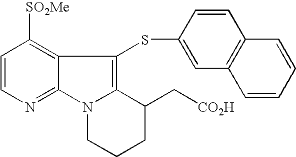

The title compound was prepared as described in Example 1 using di(2-naphthyl) disulfide.

M/z 467 (M+1).

### DP Example 11

### [5-[(2,3-Dichlorophenyl)thio]-4-(methylsulfonyl)-6,7,8,9-tetrahydropyrido[3,2-b]indolizin-6-yl]acetic acid (Compound Q)

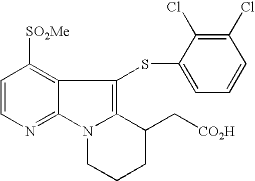

The title compound was prepared as described in Example 1 using bis(2,3-dichlorophenyl)disulfide.

¹H NMR (500 MHz, acetone-d₆) δ 8.85 (d, 1H), 7.80 (d, 1H), 7.30 (d, 1H), 7.00 (t, 1H), 6.60 (d, 1H), 4.60 (m, 1H), 4.20 (m, 1H), 3.80 (m, 1H), 3.40 (s, 3H), 2.80 to 2.10 (m, 6H).

### DP Example 12

### [5-[(4-Methylphenyl)thio]-4-(methylsulfonyl)-6,7,8,9-tetrahydropyrido[3,2-b]indolizin-6-yl]acetic acid (Compound R)

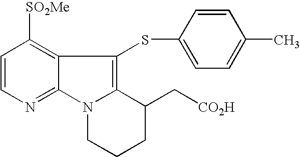

The title compound was prepared as described in Example 1 using p-tolyl disulfide.

¹H NMR (500 MHz, acetone-d₆) δ 8.55 (d, 1H), 7.80 (d, 1H), 6.95 (m, 4H), 4.60 (m, 1H), 4.15 (m, 1H), 3.80 (m, 1H), 3.35 (s, 3H), 2.80 to 2.10 (m, 6H).

### DP Example 13

### [4-(Methylsulfonyl)-5-(phenylthio)-6,7,8,9-tetrahydropyrido[3,2-b]indolizin-6-yl]acetic acid (Compound S)

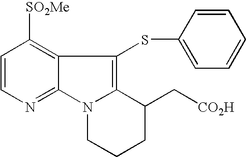

The title compound was prepared as described in Example 1 using diphenyl disulfide.

¹H NMR (500 MHz, acetone-d₆) δ 8.55 (d, 1H), 7.80 (d, 1H), 7.15 to 6.90 (m, 5H), 4.60 (m, 1H), 4.15 (m, 1H), 3.75 (m, 1H), 3.30 (s, 3H), 2.80 to 2.10 (m, 6H).

### DP Example 14

### [5-[(2,4-Dichlorophenyl)thio]-4-(methylsulfonyl)-6,7,8,9-tetrahydropyrido[3,2-b]indolizin-6-yl]acetic acid (Compound T)

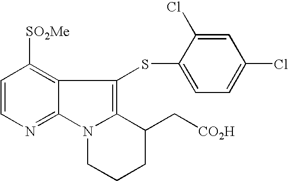

The title compound was prepared as described in Example 1 using bis(2,4-dichlorophenyl)disulfide. The disulfide was prepared from 2,4-dichlorothiophenyl using Br₂ in ether.

¹H NMR (500 MHz, acetone-d₆) δ 8.55 (d, 1H), 7.85 (d, 1H), 7.35 (s, 1H), 7.00 (d, 1H), 6.65 (d, 1H), 4.55 (m, 1H), 4.15 (m, 1H), 3.80 (m, 1H), 3.35 (s, 3H), 2.80 to 2.10 (m, 6H).

### DP Example 15

### [5-[(4-Chlorophenyl)thio]-4-(methylsulfonyl)-6,7,8,9-tetrahydropyrido[4,3-b]indolizin-6-yl]acetic acid (Compound U)

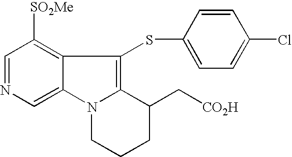

The title compound was prepared as described in Example 1 from 3-chloronicotinaldehyde (Heterocycles p. 151, 1993) except the terminal cyclization was performed by adding the azide to decalin at reflux.

¹H NMR (500 MHz, acetone-d₆) δ 9.20 (s, 1H), 8.85 (s, 1H), 7.20 (d, 2H), 7.00 (d, 2H), 4.70 (m, 1H), 4.30 (m, 1H), 3.75 (m, 1H), 3.35 (s, 3H), 2.80 to 2.10 (m, 6H).

### DP Example 16

### [9-[(4-Chlorophenyl)thio]-1-(methylsulfonyl)-7,8-dihydro-6H-pyrido[3,4-b]pyrrolizin-8-yl]acetic acid (Compound V)

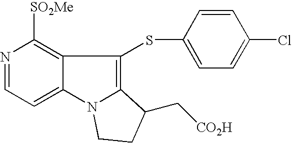

The title compound was prepared from the product of Example 6 Method 1 Step 8, as described in the procedures outlined in Example 1 Steps 10 and 11, using bis(4-chlorophenyl)disulfide in Step 10.

1H NMR (500 MHz, acetone-d₆) δ 8.25-8.3 (m, 1H), 7.71-7.75 (m, 1H), 7.12-7.17 (m, 2H), 6.97-7.04 (m, 2H), 4.45-4.51 (m, 1H), 4.32-4.39 (m, 1H), 3.73-3.80 (m, 1H), 3.29 (s, 3H), 3.15-3.21 (m, 1H), 2.99-3.08 (m, 1H), 2.66-2.73 (m, 1H), 2.46-2.54 (m, 1H).

### DP Example 17

### (−)-[(4-Chlorobenzyl)-7-fluoro-5-methanesulfonyl)-1,2,3,4-tetrahydrocyclopenta[b]indol-3-yl]acetic acid (Compound E)

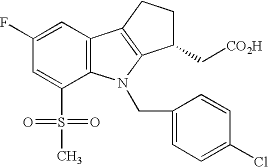

### Step 1: (+/−)-(7-Fluoro-1,2,3,4-tetrahydrocyclopenta[b]indol-3-yl)acetic acid ethyl ester

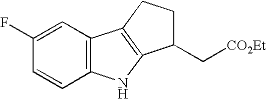

A solution of 10.00 g of 4-fluoro-2-iodoaniline, 6.57 g of ethyl 2-(2-oxocyclopentyl)acetate and 121 mg of p-toluenesulfonic acid in 100 ml of benzene was refluxed with a Dean-Stark trap under a N₂ atmosphere for 24 h. After this time, the benzene was removed under distillation. Then, 60 ml of DMF was added and the solution was degassed before 19 ml of Hunig's base followed by 405 mg of Pd(OAc)₂ were added successively. The solution was heated to 115° C. for 3 h, then cooled to room temperature. To quench the reaction, 300 ml of 1 N HCl and 200 ml of ethyl acetate were added and the mixture was filtered through Celite. The phases were separated and the acidic phase was extracted twice with 200 ml of ethyl acetate. The organic layers were combined, washed with brine, dried over anhydrous Na₂SO₄, filtered through Celite and concentrated. The crude material was further purified by flash chromatography eluting with 100% toluene, to provide the title compound.

¹H NMR (acetone-d₆) δ 9.76 (br s, 1H), 7.34 (dd, 1H), 7.03 (d, 1H), 6.78 (td, 1H), 4.14 (q, 2H), 3.57 (m, 1H), 2.85-2.55 (m, 5H), 2.15 (m, 1H), 1.22 (t, 3H).

### Step 2: (+/−)-(7-Fluoro-1,2,3,4-tetrahydrocyclopenta[b]indol-3-yl)acetic acid

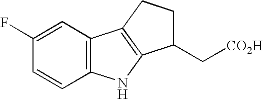

To a solution of 1.24 g of the ester from Step 1 in 14 mL of tetrahydrofuran (THF) at room temperature, 7 mL of MeOH followed by 7 mL of 2N NaOH were added. After 2.5 h, the reaction mixture was poured into a separatory funnel containing ethyl acetate (EtOAc)/1N HCl. The phases were separated and the acidic phase was extracted twice with EtOAc. The organic layers were combined, washed with brine, dried over anhydrous Na₂SO₄ and evaporated to dryness to yield a crude oil that was used as such in the next step (>90% purity).

¹H NMR (acetone-d₆) δ 10.90 (br s, 1H), 9.77 (br s, 1H), 7.34 (dd, 1H), 7.04 (dd, 1H), 6.79 (td, 1H), 3.56 (m, 1H), 2.90-2.50 (m, 5H), 2.16 (m, 1H). MS (−APCI) m/z 232.2 (M−H)⁻.

### Step 3: (+/−)-(5-bromo-7-fluoro-1,2,3,4-tetrahydrocyclopenta[b]indol-3-yl)acetic acid

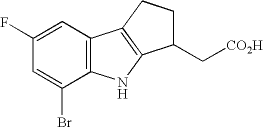

To a solution of 2.20 g of the acid from Step 2 (>90% purity) in 30 mL of pyridine, 6.85 g of pyridinium tribromide (90% purity) was added at −40° C. The suspension was stirred for 10 min at 0° C. and warmed to room temperature for 30 min. Then, the solvent was removed without heating under high vacuum. The crude material was dissolved in 40 mL of AcOH and 2.88 g of Zn dust was added portion wise to the cold solution at 0° C. The suspension was stirred for 15 min at 15° C. and warmed to room temperature for an additional 15 min. At this time, the reaction mixture was quenched by the addition of 1N HCl and this mixture was poured into a separatory funnel containing brine/EtOAc. The layers were separated and the organic layer was washed with water, brine, dried over anhydrous Na₂SO₄ and concentrated. This material was used without further purification in the next step.

¹H NMR (acetone-d₆) δ 10.77 (br s, 1H), 9.84 (br s, 1H), 7.09 (m, 2H), 3.60 (m, 1H), 2.95-2.65 (m, 4H), 2.56 (dd, 1H), 2.19 (m, 1H).

### Step 4: (+/−)-[5-bromo-4-(4-chlorobenzyl)-7-fluoro-1,2,3,4-tetrahydrocyclopenta[b]indol-3-yl]-acetic acid

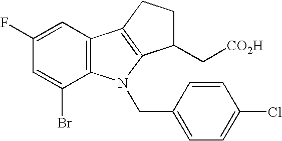

To a solution of 2.13 g of the acid from Step 3 in 10 mL of THF, a solution of diazomethane in ether was added in excess until complete consumption of the acid as monitored on TLC. Then, the solvents were removed under vacuum. To a solution of the crude methyl ester thus formed in 20 mL of DMF, 539 mg of a NaH suspension (60% in oil) was added at −78° C. The suspension was stirred for 10 min at 0° C., cooled again to −78° C. and treated with 1.70 g of 4-chlorobenzyl bromide. After 5 min, the temperature was warmed to 0° C. and the mixture was stirred for 20 min. At this time, the reaction was quenched by the addition of 2 mL of AcOH and this mixture was poured into a separatory funnel containing 1N HCl/EtOAc. The layers were separated and the organic layer was washed with brine, dried over anhydrous Na₂SO₄ and concentrated. The alkylated material was hydrolyzed using the procedure described in Step 2. The crude material was further purified by trituration with EtOAc/hexanes to provide the title compound.

¹H NMR (acetone-d₆) δ 10.70 (br s, 1H), 7.31 (d, 2H), 7.18 (d, 1H), 7.06 (d, 1H), 6.92 (d, 2H), 5.90 (d, 1H), 5.74 (d, 1H), 3.61 (m, 1H), 3.00-2.70 (m, 3H), 2.65 (dd, 1H), 2.39 (dd, 1H), 2.26 (m, 1H). MS (−APCI) m/z 436.3, 434.5 (M−H)⁻.

### Step 5: (+)-[5-bromo-4-(4-chlorobenzyl)-7-fluoro-1,2,3,4-tetrahydrocyclopenta[b]indol-3-yl}acetic acid

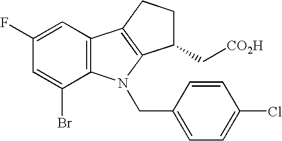

To a solution of 2.35 g of the acid of Step 4 in 130 mL of EtOH at 80° C., was added 780 μL of (S)-(−)-1-(1-naphthyl)ethylamine. The solution was cooled to room temperature and stirred overnight. The salt recovered (1.7 g) was recrystallized again with 200 mL of EtOH. After filtration, the white solid salt obtained was neutralized with 1N HCl and the product was extracted with EtOAc. The organic layer was washed with brine, dried over anhydrous Na₂SO₄ and concentrated. The material was filtered over a pad of SiO₂ by eluting with EtOAc to produce the title enantiomer. Retention times of the two enantiomers were respectively 7.5 min and 9.4 min [ChiralPak AD column, hexane/2-propanol/acetic acid (95:5:0.1)]. The more polar enantiomer was in 98% ee.

ee=98%; Retention time=9.4 min [ChiralPak AD column: 250×4.6 mm, hexanes/2-propanol/acetic acid (75:25:0.1)]; [α]_{D}²¹=+39.20 (c 1.0, MeOH).

### Step 6: (−)-[4-(4-chlorobenzyl)-7-fluoro-5-(methanesulfonyl)-1,2,3,4-tetrahydrocyclopenta[b]indol-3-yl}acetic acid and sodium salt

The acid from Step 5 (15.4 g) was first esterified with diazomethane. The sulfonylation was accomplished by mixing the ester thus formed with 16.3 g of methanesulfinic acid sodium salt and 30.2 g of CuI (I) in N-methylpyrrolidinone. The suspension was degassed under a flow of N², heated to 150° C. and stirred for 3 h, then cooled to room temperature. To quench the reaction, 500 ml of ethyl acetate and 500 ml of hexanes were added and the mixture was filtered through a pad of SiO₂ by eluting with EtOAc. The organic phases were concentrated. The crude oil was dissolved with EtOAc, washed three times with water one time with brine, dried over anhydrous Na₂SO₄, filtered and concentrated. The crude material was further purified by flash chromatography eluting with a gradient from 100% toluene to 50% toluene in EtOAc, to provide the sulfonated ester, which was hydrolyzed using the procedure described in Step 2. The title compound was obtained after two successive recrystallizations: isopropyl acetate/heptane followed by CH₂Cl₂/hexanes.

¹H NMR (500 MHz acetone-d₆) δ 10.73 (br s, 1H), 7.57 (d, 2H, J=8.8 Hz), 7.31 (m, 1H), 7.29 (m, 1H), 6.84 (d, 2H, J=8.8 Hz), 6.29 (d, 1H, J_{AB}=17.8 Hz), 5.79 (d, 1H, J_{AB}=17.8 Hz), 3.43 (m, 1H), 2.98 (s, 3H), 2.94 (m, 1H), 2.85-2.65 (m, 3H), 2.42 (dd, 1H, J₁=16.1 Hz, J₂=10.3 Hz), 2.27 (m, 1H). ¹³C NMR (125 MHz acetone-d₆) δ 173.0, 156.5 (d, J_{CF}=237 Hz), 153.9, 139.2, 133.7, 133.3, 130.0 (d, J_{CF}=8.9 Hz), 129.6, 128.2, 127.5 (d, J_{CF}=7.6 Hz), 122.2 (d, J_{CF}=4.2 Hz), 112.3 (d, J_{CF}=29.4 Hz), 111.0 (d, J_{CF}22.6 Hz), 50.8, 44.7, 38.6, 36.6, 36.5, 23.3. MS (−APCI) m/z 436.1, 434.1 (M−H)⁻. ee=97%; Retention time=15.3 min [ChiralCel OD column: 250×4.6 mm, hexanes/2-propanol/ethanol/acetic acid (90:5:5:0.2)]; [α]_{D}²¹=−29.3° (c 1.0, MeOH). Mp 175.0° C.

The sodium salt was prepared by the treatment of 6.45 g (14.80 mmol) of the above acid compound in EtOH (100 mL) with 14.80 mL of an aqueous 1N NaOH solution. The organic solvent was removed under vacuum and the crude solid was dissolved in 1.2L of isopropyl alcohol under reflux. The final volume was reduced to 500 mL by distillation of the solvent. The sodium salt crystallized by cooling to rt. The crystalline sodium salt was suspended in H₂O, frozen with a dry ice bath and lyophilized under high vacuum to give the title compound as the sodium salt.

¹H NMR (500 MHz DMSO-d₆) δ 7.63 (dd, 1H, J₁=8.5 Hz, J₂=2.6 Hz), 7.47 (dd, 1H, J₁=9.7 Hz, J₂=2.6 Hz), 7.33 (d, 2H, J=8.4 Hz), 6.70 (d, 2H, J=8.4 Hz), 6.06 (d, 1H, J_{AB}=17.9 Hz), 5.76 (d, 1H, J_{AB}=17.9 Hz), 3.29 (m, 1H), 3.08 (s, 3H), 2.80 (m, 1H), 2.69 (m, 1H), 2.55 (m, 1H), 2.18 (m, 2H), 1.93 (dd, 1H, J₁=14.4 Hz, J₂=9.7 Hz).

### DP Example 17A

### Alternative procedure for (+/−)-[5-bromo-4-(4-chlorobenzyl)-7-fluoro-1,2,3,4-tetrahydrocyclopenta[b]indol-3-yl]acetic acid (Example 17, Step 4)

### Step 1: (+/−)-7-fluoro-1,2,3,4-tetrahydrocyclopenta[b]indol-3-yl)acetic acid dicyclohexylamine (DCHA) salt

A 0.526 M solution of 2-bromo-4-fluoroaniline in xylene along with ethyl (2-oxocyclopentyl)acetate (1.5 eq) and sulfuric acid (0.02 eq) was heated to reflux for 20 hours. Water was azeotropically removed with a Dean-Stark apparatus. The reaction was followed by NMR and after 20 hours, an 80-85% conversion to the desired imine intermediate was generally observed. The reaction mixture was washed with 1M sodium bicarbonate (0.2 volumes) for 15 minutes and the organic fraction was evaporated. The remaining syrup was distilled under vacuum (0.5 mm Hg). Residual xylenes distilled at 30° C., then excess ketone and unreacted aniline were recovered in the 50-110° C. range; the imine was recovered in the 110-180° C. fraction as a light brown clear liquid with 83% purity.

The imine intermediate was then added to a degased mixture of potassium acetate (3 eq), tetra-n-butylammonium chloride monohydrate (1 eq), palladium acetate (0.03 eq) and N,N-dimethylacetamide (final concentration of imine=0.365 M). The reaction mixture was heated to 115° C. for 5 hours and allowed to cool to room temperature. 3N KOH (3 eq) was then added and the mixture was stirred at room temperature for 1 hour. The reaction mixture was diluted with water (1.0 volume), washed with toluene (3×0.75 volume). The aqueous phase was acidified to pH 1 with 3N HCl and extracted with tertbutyl methyl ether (2×0.75 volume). The combined organic fractions were washed with water (0.75 volume). To the clear light brown solution was added dicyclohexylamine (1 eq) and the solution was stirred at room temperature for 16 hours. The salt was filtered, washed with ethyl acetate, tertbutyl methyl ether and allowed to dry to give the title compound. Assay: 94 A %.

1H NMR (500 mHz, CDCl₃): δ 9.24 (s, 1H), 7.16-7.08 (m, 2H), 6.82 (t, 1H), 6.2 (br, 2H), 3.6-3.5 (m, 1H), 3.04-2.97 (m, 2H), 2.88-2.70 (m, 3H), 2.66 (dd, 1H), 2.45-2.37 (m, 1H), 2.13-2.05 (m, 2.05), 1.83 (d, 4H), 1.67 (d, 2H), 1.55-1.43 (m, 4H), 1.33-1.11 (m, 6H).

### Step 2: (+/−)-(5-bromo-7-fluoro-1,2,3,4-tetrahydrocyclopenta[b]indol-3-yl)acetic acid

A slurry of the DCHA salt from Step 1 above in dichloromethane (0.241 M solution) was cooled to −20 to −15° C. Pyridine (2 eq.) was added in one shot and to the slurry was added dropwise bromine (2.5 eq.) over 30 to 45 minutes maintaining the temperature between −20° C. and −15° C. (At about ⅓ addition of bromine, the reaction mixture was thick and an efficient stirring was needed. Eventually, at about ½ addition of bromine, the mixture became “loose” again.) After completion of the addition, the reaction mixture was aged for one additional hour at −15° C. Acetic acid (3.04 eq.) was then added over 5 minutes and zinc dust (3.04 eq.) was added portion wise. (A portion of zinc was added at −15° C. and the mixture was aged for about 5 minutes to ensure that the exotherm was going (about −15° C. to −10° C.)). This operation was repeated over about 30 min. When no more exotherm was observed, the remaining zinc was added faster. The whole operation took around 30 to 45 minutes.

After completion of the addition, the batch was warmed to room temperature, aged 1 hour and concentrated. The reaction mixture was switched to methyl t-butyl ether (MTBE, 0.8 volume) and a 10% aqueous acetic acid solution (0.8 volume) was added. The mixture (crystallization of salts, e.g. pyridium) was aged at room temperature for 1 hour and filtered through solka-floc. The pad of solka-floc was rinsed with MTBE (ca. 0.2 volume) and the filtrate (biphasic, MTBE/aqueous) was transferred into an extractor. The organic phase was washed with water (0.8 volume). The MTBE extract was concentrated and switched to isopropyl alcohol (IPA, 0.25 volume) to crystallize the compound. Water (0.25 volumes) was added and the batch was aged for 1 hour. Additional water (0.33 volumes) was added over 1 hour. After completion of the water addition, the batch was aged for one additional hour, filtered, and rinse with 30/70 IPA/Water (0.15 volumes). Crystallized bromoacid was dried in the oven at +45° C.

### Step 3: (+/−)-[5-bromo-4-(4-chlorobenzyl)-7-fluoro-1, 2,3,4-tetrahydrocyclopenta[b]indol-3-yl]-acetic acid

The bromoacid of Step 2 was dissolved in dimethylacetamide (0.416 M solution) and cesium carbonate (2.5 eq.) was added in one portion. To the slurry was added in one portion 4-chlorobenzyl chloride (2.5 eq.) and the batch was heated to 50° C. for 20 h. The batch was cooled to r.t. and sodium hydroxide 5N (4.00 eq.) was added over 5 minutes (temperature rose to +40° C.). The reaction was aged at 50° C. for ca. 3 hours, cooled to room temperature and transferred into an L extractor. The solution was diluted with isopropylacetate (IPAc, 2 volumes) and cooled to +15° C. The solution was acidified with 5N HCl to pH˜2. Layers were separated and the organic layer was washed with water (2×2 volumes). IPAc solution was concentrated and switched to IPA (0.8 volumes) to crystallize the product. Water (8 L) was added over 2 hours and the batch was filtered to give the title compound. The batch can be dried in the oven at +40° C. for 24 hours.

### DP Example 18

### (+/−)-{4-[1-(4-Chlorophenyl)ethyl]-7-fluoro-5-methanesulfonyl-1,2,3,4-tetrahydrocyclopenta[b]indol-3-yl}acetic acid (Compound X)

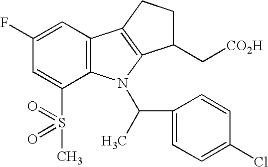

The title compound was synthesized in accordance with the description provided in PCT WO03/062200 published on Jul. 30, 2003.

### DP Example 19

### (+/−)-[9-(4-Chlorobenzyl)-6-fluoro-methanesulfonyl-2,3,4,9-tetrahydro-1H-carbazol-1-yl]acetic acid (Compound Y)

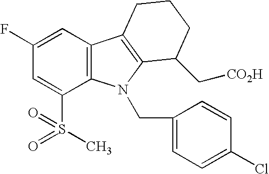

The title compound was synthesized in accordance with the description provided in PCT WO03/062200 published on Jul. 30, 2003.

### DP Example 20

### [4-(4-Chlorobenzyl)-7-fluoro-5-methanesulfonyl-1-oxo-1,2,3,4-tetrahydrocyclopenta[b]indol-3-yl]acetic acid (Compound Z)

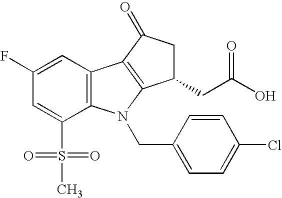

The title compound was synthesized in accordance with the description provided in PCT WO03/062200 published on Jul. 30, 2003.

### DP Example 21

### {9-[(3,4-Dichlorophenyl)thio]-1-isopropyl-7,8-dihydro-6H-pyrido[3,4-b]pyrrolizin-8-yl}acetic acid (Enantiomer A and Enantiomer B) (Compound AA)

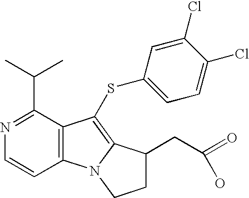

### Step 1 2-Chloronicotinaldehyde

To a solution of diisopropyl amine (110 mL, 780 mmol) in THF (500 mL) was added a 2.5 M hexanes solution of n-BuLi (300 mL, 750 mmol) at −40° C. After 5 min, the reaction mixture was cooled to −95° C. then DMPU (15 mL) and 2-chloropyridine (50 mL, 532 mmol) were successively added. The resulting mixture was then warmed and stirred at −78° C. for 4 h. After this time, the yellow suspension was cooled again to −95° C. before DMF (70 mL) was added. The final reaction mixture was warmed to −78° C. and stirred at that temperature for 1.5 h. The reaction mixture was poured into cold aqueous HCl (3N, 800 mL) and stirred for 5 min. Aqueous concentrated NH₄OH was added to adjust pH to 7.5. The aqueous layer was extracted three times with EtOAc. The combined organic layer was washed with aqueous NH₄Cl and brine, dried over anhydrous Na²SO₄, filtered and concentrated. The crude material was further purified by a pad of silica gel by eluting with a gradient from 100% hexanes to 100% EtOAc and the product was crystallized in cold hexanes to yield the title compound as a pale yellow solid.

### Step 2 Methyl (2Z)-2-azido-3-(2-chloropyridin-3-yl)prop-2-enoate

A solution of 2-chloronicotinealdehyde (20.0 g, 139.9 mmol) and methyl azidoacetate (32.2 mL, 349.7 mmol) in MeOH (168 mL) was added to a solution of 25% NaOMe in MeOH (80 mL, 349 mmol) at −20° C. The internal temperature was monitored and maintained at −20° C. during the 30 min. addition. The resulting mixture was then stirred in an ice bath for several hours, followed by overnight in an ice bath in the cold room. The suspension was then poured onto a mixture of ice and NH₄Cl, and the slurry was filtered after 10 min. of stirring. The product was washed with cold H₂O and was then dried under vacuum. The crude material was dissolved in CH₂Cl₂ and MgSO₄ was added. The suspension was filtered through a pad of silica gel, washed with CH₂Cl₂. The filtrate was concentrated under reduced pressure and a beige precipitate (20 g) of the title product was obtained.

### Step 3 Methyl 4-chloro-1H-pyrrolo[3,2-c]pyridine-2-carboxylate

A solution of methyl (2Z)-2-azido-3-[2-chloropyridin-3-yl]prop-2-enoate (21 g, 88 mmol) in mesitylene (880 mL) was heated at reflux for a period of 1 h. The reaction mixture was cooled to room temperature then to 0° C., and the precipitate was filtered and washed with cold hexane. The material was stirred overnight in 1:20 EtOAc/hexane to give, after filtration, the title product as a pale yellow solid.

### Step 4 Methyl 1-chloro-8-oxo-7,8-dihydro-6H-pyrido[3,4-b]pyrrolizine-7-carboxylate

To a suspension of methyl 4-chloro-1H-pyrrolo[3,2-c]pyridine-2-carboxylate (12.5 g, 59 mmol) in THF (116 mL)—toluene (460 mL) were added a 1.0 M THF solution of potassium tert-butoxide (64 mL, 64 mmol) and methyl acrylate (55 mL, 611 mmol). The resulting mixture was heated at 100° C. for 18 h. After this time, the suspension was cooled to room temperature and it was poured into a mixture of saturated aqueous NH₄Cl (400 mL) and hexanes (400 mL). The solids were decanted, filtered and washed with H₂O and hexanes to provide the title compound.

### Step 5 1-Chloro-6,7-dihydro-8H-pyrido[3,4-b]pyrrolizin-8-one

To the compound of the previous step were added isopropanol (8.0 mL) and concentrated HCl (2.0 mL) with heating at 100° C. for 1 h. The reaction mixture was partitioned between EtOAc and Na₂CO₃. The organic phase was separated, evaporated to provide the title compound.

### Step 6 1-Isopropenyl-6,7-dihydro-8H-pyrido[3,4-b]pyrrolizin-8-one

To a mixture of 1-chloro-6,7-dihydro-8H-pyrido[3,4-b]pyrrolizin-8-one (5.0 g, 24.3 mmol), tris(dibenzylidene acetone)dipalladium (0) (1.0 g, 1.09 mmol) and triphenylarsine (2.70 g, 8.82 mmol) in DMF (100 mL) was added tributylisopropenyl stannane (9.60 g, 29.00 mmol). The resulting mixture was degassed and heated at 78° C. for a period of 18 h. The solvent was evaporated under reduced pressure. CH₂Cl₂ and celite were added to the resulting mixture which was then filtered over celite. The title compound was purified by flash chromatography (50% to 100% EtOAc in Hexane).

### Step 7 Ethyl (2E)-(1-isopropenyl-6,7-dihydro-8H-pyrido[3,4-b]pyrrolizin-8-ylidene)ethanoate

To a solution of 1-isopropenyl-6,7-dihydro-8H-pyrido[3,4-b]pyrrolizin-8-one (0.60 g, 2.8 mmol) and triethyl phosphonoacetate (1.00 g, 4.46 mmol) in THF (24 mL) at −78° C. was added 80% NaH (0.12 g, 4.00 mmol), the reaction mixture was allowed to warm to 0° C., then to room temperature. The reaction mixture was poured onto saturated NH₄Cl and EtOAc. The organic phase was separated, dried over Na₂SO₄ and evaporated. The title compound was purified by flash chromatography (40% EtOAc in Hexane).

### Step 8 Ethyl (1-isopropyl-7,8-dihydro-6H-pyrido[3,4-b]pyrrolizin-8-yl)acetate

To a solution of ethyl (2E)-(1-isopropenyl-6,7-dihydro-8H-pyrido[3,4-b]pyrrolizin-8-ylidene)ethanoate (0.40 g, 1.4 mmol) in MeOH (20 mL) was added Pd(OH)₂ (0.20 g). The mixture was stirred under 1 atm of H₂ for 3 h. The mixture was filtered over celite and evaporated to provide the title compound.

Step 9 Ethyl {9-[(3,4-dichlorophenyl)thio]-1-isopropyl-7,8-dihydro-6H-pyrido[3,4-b]pyrrolizin-8-yl}acetate

To a solution of bis(3,4-dichlorophenyl)disulfide (0.24 g, 0.67 mmol) in CH₂Cl₂ (5.6 mL) was added SO₂Cl₂ (0.036 mL). The resulting yellow mixture was stirred at room temperature for 1 h. This solution was added to a solution of ethyl (1-isopropyl-7,8-dihydro-6H-pyrido[3,4-b]pyrrolizin-8-yL) acetate (0.15 g, 0.52 mmol) in DMF (5.6 mL) at 0° C. After 1.5 h at 0° C., the reaction mixture was poured over saturated NaHCO₃ and EtOAc. The organic phase was separated, dried over Na₂SO₄, filtered and evaporated. The title compound was purified by flash chromatography (30% to 40% EtOAc in Hexane).

### Step 10 {9-[(3, 4-Dichlorophenyl)thio]-1-isopropyl-7,8-dihydro-6H-pyrido[3,4-b]pyrrolizin-8-yl}acetic acid

To a solution of ethyl {9-[(3,4-dichlorophenyl)thio]-1-isopropyl-7,8-dihydro-6H-pyrido[3,4-b]pyrrolizin-8-yl}acetate (0.23 g, 0.50 mmol) in THF (5 mL and MeOH (2.5 mL) was added 1.0 M NaOH (1.5 mL, 1.5 mmol). After stirring 18 h at RT, HOAc (0.25 mL) was added and the solvent was evaporated. The residue was taken up in EtOAc/H₂O, and the organic layer was washed with H₂O and brine. After drying (Na₂SO₄), the solution was filtered and evaporated. The residue was stirred with 1:1 EtOAc:hex to give, after filtration, the title compound as a white solid.

¹H NMR (MeOH-d₄) δ 1.14-1.26 (m, 6H), 2.47-2.56 (m, 1H), 2.56-2.64 (m, 1H), 2.94-3.05 (m, 2H), 3.81-3.89 (m, 1H), 4.22-4.30 (m, 1H), 4.33-4.44 (m, 2H), 6.93-6.99 (m, 1H), 7.14-7.19 (m, 1H), 7.33-7.39 (m, 1H), 7.54-7.59 (m, 1H), 8.16-8.21 (m, 1H).

The product of Step 10 was converted to its methyl ester using CH₂N₂, and the ester was subjected to HPLC separation on chiral stationary phase (chiralcel OD column 2×25 cm), eluting with 12% 2-propanol in hexane at a flow rate of 6 mL/min. Enantiomer A (less polar) has a retention time of 31.9 min and Enantiomer B (more polar) has a retention time of 35.5 min. Both A and B were hydrolyzed as in Ex. 17 Step 10 to give enantiomers A and B of the title compound.

### DP Example 22

### ((1R)-6-Fluoro-8-(methylsulfonyl)-9-{(1S)-1-[4-(trifluoromethyl)phenyl]ethyl}-2,3,4,9-tetrahydro-1H-carbazol-1-yl)acetic acid (Compound AJ)

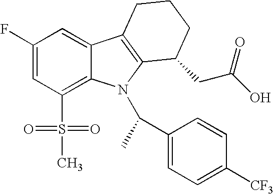

### Step 1: 2-(2-Bromo-4-fluorophenyl)hydrazinium chloride

To a suspension of 2-bromo-4-fluoroaniline in concentrated HCl (1.5M) at −10° C. was slowly added a 10.0M aqueous solution of NaNO₂ (1.1 eq). The mixture was stirred at 0° C. for 2.5 hrs. A cold (−30° C.) solution of SnCl₂ (3.8M) in concentrated HCl was then slowly added while maintaining the internal temperature below 10° C. The resulting mixture was stirred mechanically for 20 min at 10° C., then at room temperature for 1 hr. The thick slurry was filtered and the solid was air dried overnight. The solid was resuspended in cold HCl and filtered again. The dried material was suspended in Et₂O, stirred for 10 min, filtered and air dried overnight to give the title compound as a beige solid.

### Step 2: (+/−)-Ethyl (8-bromo-6-fluoro-2,3,4,9-tetrahydro-1H-carbazol-1-yl)acetate

To a suspension of the compound of Step 1 (1 eq) in AcOH (0.5M) was added ethyl (2-oxocyclohexyl)acetate (1 eq). The mixture was stirred at reflux for 16 hrs, cooled and AcOH was removed by evaporation under reduced pressure. The residue was diluted with EtOAc and washed with water and saturated aqueous NaHCO₃. The organic layer was dried over Na₂SO₄ and concentrated. The residue was then purified on a pad of silica gel, eluting with toluene. The filtrate was concentrated and stirred in hexanes to give, after filtration, the title compound as a white solid. MS (+APCI) m/z 354.2 (M+H)⁺.

### Step 3: (+/−)-Ethyl[6-fluoro-8-(methylsulfonol)-2,3,4,9-tetrahydro-1H-carbazol-1-yl]-acetate

To a solution of the compound of Step 2 (1 eq) in anhydrous DMSO (0.28M) were added sodium methanesulphinate (3 eq) and copper iodide (3 eq). N₂ was bubbled into the mixture for 5 min and the reaction was then stirred at 100° C. under N₂ atmosphere. After 12 hrs, more sodium methanesulphinate (2 eq) and copper iodide (2 eq) were added. The mixture was stirred for a further 12 hrs at 100° C., cooled, diluted with EtOAc and 1N HCl was added to acidify the mixture. The suspension was stirred for 30 min and filtered through celite. The filtrate was washed with water, dried over Na₂SO₄ and concentrated. The residue was filtered through a pad of silica gel, eluting first with toluene to remove the non-polar impurities and then with a 2:1 mixture of hexanes/EtOAc to elute the desired product. The filtrate from the elution with the mixture of hexanes/EtOAc was concentrated to give the title compound as a pale yellow solid. MS (−APCI) m/z 352.1 (M−H)

### Step 4: Ethyl[(1R)-6-fluoro-8-(methylsulfonyl)-2,3,4,9-tetrahydro-1H-carbazol-1-yl]acetate

The racemic mixture from step 3 was resolved by preparative HPLC on a chiralpak AD preparative column eluted with a mixture of 15% iPrOH in hexane. The more polar enantiomer (longer retention time) was identified as the title compound based on the activity of the final product.

### Step 5: Ethyl[(1R)-9-[(1S)-1-(4-chlorophenyl)ethyl]-6-fluoro-8-(methylsulfonyl)-2,3,4,9-tetrahydro-1H-carbazol-1-yl]acetate

To a solution of the compound of Step 4 (1 eq), triphenylphosphine (1.5 eq) and (1R)-1-(4-chlorophenyl)ethanol (1.5 eq, prepared following the general procedure described in Reference Example 1) in THF (0.175M) was added a solution of di-tert-butyl azodicarboxylate (2.1 M in THF, 1.5 eq) over a 10 min period. The mixture was stirred at room temperature for 2 hr and concentrated. The residue was purified by silica gel flash chromatography, eluting with 7% EtOAc in toluene to give the desired product (˜90% pure) which was used as such for the next reaction.

### Step 6: [(1R)-9-[(1S)-1-(4-Chlorophenyl)ethyl]-6-fluoro-8-(methylsulfonyl)-2,3,4,9-tetrahydro-1H-carbazol-1-yl]acetic acid and [(1S)-9-[(1S)-1-(4-chlorophenyl)ethyl]-6-fluoro-8-(methylsulfonyl-2,3,4,9-tetrahydro-1H-carbazol-1-yl]acetic acid

To a solution of the compound of Step 5 in a 2:1 mixture of THF and methanol (0.1M) was added 1N aqueous LiOH (3 eq). The mixture was stirred at room temperature for 2 hr, AcOH was added and the solvent was removed by evaporation. The residue was taken up in EtOAc/H₂O and the organic layer was washed with brine, dried over Na₂SO₄, filtered and concentrated. The residue was swished in 30% EtOAc in hexane, and the product was suspended in diethyl ether and sonicated for 45 min, filtered, and dried under high vacuum at 50° C. for 24 hr to give the title compound as a white solid. MS (−APCI) m/z 462.1 (M−H)

Alternatively (+/−) ethyl[6-fluoro-8-(methylsulfonyl)-2,3,4,9-tetrahydro-1H-carbazol-1-yl]acetate was used for the alkylation reaction in step 5 to give a mixture of 2 diastereomers: ethyl[(1R)-9-[(1S)-(4-chlorophenyl)ethyl]-6-fluoro-8-(methylsulfonyl)-2,3,4,9-tetrahydro-1H-carbazol-1-yl]acetate and ethyl[(1S)-9-[(1S)-1-(4-chlorophenyl)ethyl]-6-fluoro-8-(methylsulfonyl)-2,3,4,9-tetrahydro-1H-carbazol-1-yl]acetate. The diastereomeric mixture was resolved by selective hydrolysis using the following procedure to give the desired [(1R)-9-[(1S)-1-(4-chlorophenyl)ethyl]-6-fluoro-8-(methylsulfonyl)-2,3,4,9-tetrahydro-1H-carbazol-1-yl]acetic acid.

### Resolution:

The diastereomeric mixture of ethyl[(1R)-9-[(1S)-1-(4-chlorophenyl)ethyl]-6-fluoro-8-(methylsulfonyl)-2,3,4,9-tetrahydro-1H-carbazol-1-yl]acetate and ethyl[(1S)-9-[(1S)-1-(4-chlorophenyl)ethyl]-6-fluoro-8-(methylsulfonyl)-2,3,4,9-tetrahydro-1H-carbazol-1-yl]acetate (1 eq) was dissolved in a 3.5/1 mixture of THF/MeOH (0.25M) and cooled at 0° C. Aqueous LiOH 1N (1 eq) was slowly added and the mixture was stirred at 0° C. for 12 h or until almost complete hydrolysis of ethyl [(1R)-9-[(1S)-1-(4-chlorophenyl)ethyl]-6-fluoro-8-(methylsulfonyl)-2,3,4,9-tetrahydro-1H-carbazol-1-yl]acetate, the other diastereomer was only slightly hydrolyzed under these conditions. AcOH was added and the solvent was removed by evaporation. The residue was taken up in EtOAc/H₂O and the organic layer was washed with brine, dried over Na₂SO₄, filtered and concentrated. Ethyl[(1S)-9-[(1S)-1-(4-chlorophenyl)ethyl]-6-fluoro-8-(methylsulfonyl)-2,3,4,9-tetrahydro-1H-carbazol-1-yl]acetate and [(1R)-9-[(1S)-(4-chlorophenyl)ethyl]-6-fluoro-8-(methylsulfonyl)-2,3,4,9-tetrahydro-1H-carbazol-1-yl]acetic acid were separated by flash chromatography eluting with 40% EtOAc in hexanes containing 1% AcOH to give the desired [(1R)-9-[(1S)-1-(4-chlorophenyl)ethyl]-6-fluoro-8-(methylsulfonyl)-2,3,4,9-tetrahydro-1H-carbazol-1-yl]acetic acid with de>90% which was swished in 30% EtOAc in hexane to give the desired compound as a white solid with de>95%.

### Step 7: Methyl[(1R)-6-fluoro-8-(methylsulfonyl)-2,3,4,9-tetrahydro-1H-carbazol-1-yl]acetate

To a solution of [(1R)-9-[(1S)-1-(4-chlorophenyl)ethyl]-6-fluoro-8-(methylsulfonyl)-2,3,4,9-tetrahydro-1H-carbazol-1-yl]acetic acid ([α]_{D}=−226° in MeOH) in MeOH (0.1M) was added 10% palladium on carbon (10% wt/wt). A stream of N2 was bubbled through the mixture for 5 min. The reaction was stirred at rt under H₂ atmosphere(balloon) for 24 hrs and filtered through a celite pad eluted with CH₂Cl₂. The solvents were removed by evaporation under reduced pressure and the residue was swished in MeOH to give the compound methyl[(1R)-6-fluoro-8-(methylsulfonyl)-2,3,4,9-tetrahydro-1H-carbazol-1-yl]acetate.

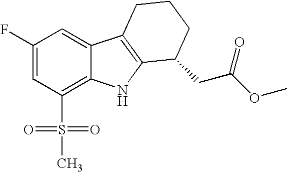

### Step 8: ((1R)-6-Fluoro-8-(methylsulfonyl)-9-{(1S)-1-[4-(trifluoromethyl)phenyl]ethyl}-2,3,4,9-tetrahydro-1H-carbazol-1-yl)acetic acid (Compound AJ)

To a solution of the compound of step 7 (1 eq), triphenylphosphine (1.5 eq) and (1R)-1-[4-(trifluoromethyl)phenyl]ethanol (1.5 eq) in THF (0.2M) was added a solution of di-tert-butyl azodicarboxylate (1M in THF, 1.5 eq) over a 20 min period. The mixture was stirred at room temperature for 2 hr and concentrated. The residue was purified by silica gel flash chromatography eluted with 10% EtOAc in toluene to give methyl((1R)-6-fluoro-8-(methylsulfonyl)-9-{(1S)-1-[4-(trifluoromethyl)phenyl]ethyl}-2,3,4,9-tetrahydro-1H-carbazol-1-yl)acetate (−90% pure) which was used as such for the next reaction.

To a solution of the above ester (1 eq) in a 3.5/1 mixture of THF/MeOH (0.25M) at 0° C. was slowly added aqueous LiOH 1N (1 eq) and the mixture was stirred at 0° C. for 16 h or until almost complete hydrolysis of the ester; under these conditions, the other minor diastereomer has a much slower rate of hydrolysis. AcOH was added and the solvent was removed in vacuo. The residue was taken up in EtOAc/H₂O and the organic layer was washed with brine, dried over Na₂SO₄, filtered and concentrated. To remove the unreacted methyl ester, the residue was filtered through a pad of silica gel eluting first with 10% EtOAc/toluene and then with 60% EtOAc/toluene containing 1% of AcOH. The residue was swished in 30% EtOAc/hexane and dried under high vacuum at 50° C. for 16 hr to give the title compound as a white solid with de and ee >95% (checked by chiral HPLC). MS (−APCI) m/z 496.0 (M−H)⁻. [α]_{D}=−181° in MeOH

### Biological Activity Assays

The activity of the compounds of formula I can be demonstrated using the following assays:

### ³H-Nicotinic Acid Competition Binding Assay

CHO-KI cells stably expressing the niacin receptor were used to make membrane for binding analysis. Cells were grown to −80% confluence in growth medium (F-12 Kaighn's modified medium (ATCC, #30-2004) containing 10% FBS (GIBCO, #10438-026), 1 mg/ml G418 (GIBCO, #10131-027) and 1×Pen-Strep (Sigma P-0871), harvested by scraping, and centrifuged at 12 000×g, 4° Celsius, 10 minutes. Cell pellets were resuspended in harvest buffer (20 mM HEPES, 10 mM EDTA, pH 7.4) and homogenized with 4×10 second bursts of a 12 mm Polytron homogenizer, setting 5. Lysate was centrifuged at 2 000×g, 4°, 10 minutes to remove unlysed cells and nuclei, and the resulting supernatant centrifuged at 39 000×g, 4°, 45 minutes to pellet membranes. The resulting pellet was resuspended in wash buffer (20 mM HEPES, 0.1 mM EDTA, pH 7.4), homogenized with 3×10 second bursts of a 12 nun Polytron, setting 4, and re-centrifuged at 39 000×g, 4°, 45 minutes. The resulting pellet was resuspended in wash buffer and stored in liquid nitrogen before use. The concentration of membrane proteins in this preparation was determined using the Pierce BCA protein assay, with BSA as a standard.

Equilibrium binding of ³H-nicotinic acid was performed in 96-well polypropylene plates. Reactions contained 140 μl membrane diluted in assay buffer (20 mM HEPES, pH 7.4, 1 mM MgCl₂, and 0.01% CHAPS; 15-30 μg membrane protein/assay), 20 μl test compounds diluted in assay buffer (compound stocks were in 100% DMSO; final DMSO concentration in the assay was 0.25%), and 40 μl 250 nM tritiated niacin ([5, 6-³H]—nicotinic acid: American Radiolabeled Chemicals, Inc., 20 μM in ethanol; final ethanol concentration in each assay was 1.5%). Non-specific binding was determined in the presence of 250 μM unlabeled nicotinic acid. After mixing at 3-4 hours at room temperature, reactions were filtered through Packard Unifilter GF/C plates using a Packard Harvester, and washed with 8×200 μl ice-cold binding buffer. Plates were dried overnight and their backs sealed using PerkinElmer tape designed for GF/C plates. 40 μl PerkinElmer Microscint-20 scintillation fluid was added to each well, the tops sealed, and plates analyzed in a Packard TopCount scintillation counter.

Calculations: The test compounds are initially assayed at 1 and 0.1 μM and then at a range of concentrations chosen such that the middle dose would cause about 50% inhibition of a Radio-Ligand binding (i.e., IC₅₀). Specific binding in the absence of test compound (B_{O}) is the difference of total binding (B_{T}) minus non-specific binding (NSB) and similarly specific binding (in the presence of test compound) (B) is the difference of displacement binding (B_{D}) minus non-specific binding (NSB). IC₅₀ is determined from an inhibition response curve, logit-log plot of % B/B_{O} vs concentration of test compound.

Kᵢ is calculated by the Cheng and Prustoff transformation:

Kᵢ=IC₅₀/(1+[L]/K_{D})

where [L] is the concentration of a Radio-Ligand used in the assay and K_{D} is the dissociation constant of a Radio-Ligand determined independently under the same binding conditions.

Certain compounds of formula I have an IC₅₀ in this niacin binding assay within the range of about 0.010-50 μM. More advantageous compounds of the invention have an IC₅₀ value in this assay within the range of about 0.01-10 μM. Still more advantageous compounds have an IC₅₀ value in this assay within the range of about 0.010-1.0 μM.

### ³⁵S-GTPγS Binding Assay

Membranes prepared from Chinese Hamster Ovary (CHO)-K1 cells stably expressing the niacin receptor or vector control (7 μg/assay) were diluted in assay buffer (100 mM HEPES, 100 mM NaCl and 10 mM MgCl₂, pH 7.4) in Wallac Scintistrip plates and pre-incubated with test compounds diluted in assay buffer containing 40 μM GDP (final [GDP] was 10 μM) for ˜10 minutes before addition of ³⁵S-GTPγS to 0.3 nM. To avoid potential compound precipitation, all compounds were first prepared in 100% DMSO and then diluted with assay buffer resulting in a final concentration of 3% DMSO in the assay. Binding was allowed to proceed for one hour before centrifuging the plates at 4000 rpm for 15 minutes at room temperature and subsequent counting in a TopCount scintillation counter. Non-linear regression analysis of the binding curves was performed in GraphPad Prism.

### Membrane Preparation:

### Materials:

CHO-K1 cell culture medium: F-12 Kaighn's Modified Cell Culture Medium with 10% FBS, 2 mM L-Glutamine, 1 mM Sodium Pyruvate and 400 μ\tilde{g}ml G418

| | |
|---|---|
| Membrane Scrape Buffer: | 20 mM HEPES |
| | 10 mM EDTA, pH 7.4 |
| Membrane Wash Buffer: | 20 mM HEPES |
| | 0.1 mM EDTA, pH 7.4 |
| Protease Inhibitor Cocktail: | P-8340, (Sigma, St. Louis, MO) |

### Procedure:

Aspirate cell culture media off the 15 cm² plates, rinse with 5 mL cold PBS and aspirate.
Add 5 mL Membrane Scrape Buffer and scrape cells. Transfer scrape into 50 mL centrifuge tube. Add 50 μL Protease Inhibitor Cocktail.
Spin at 20,000 rpm for 17 minutes at 4° C.
Aspirate off the supernatant and resuspend pellet in 30 mL Membrane Wash Buffer. Add 50 μL Protease Inhibitor Cocktail.
Spin at 20,000 rpm for 17 minutes at 4° C.
Aspirate the supernatant off the membrane pellet. The pellet may be frozen at −80° C. for later use or it can be used immediately.

### Assay:

### Materials:

Guanosine 5′-diphosphate sodium salt (GDP, Sigma-Aldrich Catalog #87127)
Guanosine 5′-[γ³⁵S] thiotriphosphate, triethylammonium salt ([³⁵S]GTPγS, Amersham Biosciences

Catalog #SJ1320, ˜1000 Ci/mmol)

96 well Scintiplates (Perlcin-Elmer #1450-501)

### Binding Buffer: 20 mM HEPES, pH 7.4

100 mM NaCl

10 mM MgCl₂

GDP Buffer: binding buffer plus GDP, ranging from 0.4 to 40 μM, make fresh before assay

### Procedure:

(total assay volume=100μ/well)
25 μL GDP buffer with or without compounds (final GDP 10 μM—so use 40 μM stock)
50 μL membrane in binding buffer (0.4 mg protein/mL)
25 μL [³⁵S]GTPγS in binding buffer. This is made by adding 5 μl [³⁵S]GTPγS stock into 10 mL binding buffer (This buffer has no GDP)
Thaw compound plates to be screened (daughter plates with 5 μL compound@2 mM in 100% DMSO)
Dilute the 2 mM compounds 1:50 with 245 μL GDP buffer to 40 μM in 2% DMSO. Thaw frozen membrane pellet on ice
Homogenize membranes briefly until in suspension using a POLYTRON PT3100 (probe PT-DA 3007/2 at setting of 7000 rpm). Determine the membrane protein concentration by Bradford assay. Dilute membrane to a protein concentrations of 0.40 mg/ml in Binding Buffer. (Note: the final assay concentration is 20 μg/well).
Add 25 μL compounds in GDP buffer per well to Scintiplate.
Add 50 μL of membranes per well to Scintiplate.
Pre-incubate for 5-10 minutes at room temperature.
Add 25 μL of diluted [³⁵S]GTPγS. Incubate on shaker (Lab-Line model #1314, shake at setting of 4) for 60 minutes at room temperature.
Assay is stopped by spinning plates sealed with plate covers at 2500 rpm for 20 minutes at 22° C.
Read on TopCount NXT scintillation counter −35S protocol.

Certain compounds of formula I have an EC₅₀ in this functional GTPγS binding assay within the range of about 0.010-100 μM. More advantageous compounds of the invention have an EC₅₀ value in this assay within the range of about 0.010-10 μM. Still more advantageous compounds have an EC₅₀ value in this assay of less than about 1 μM, about 0.01-1 μM.

### Flushing via Laser Doppler

Procedure—Male C57B16 mice (−25 g) are anesthetized using 10 mg/ml/kg Nembutal sodium. When antagonists are to be administered they are co-injected with the Nembutal anesthesia. After ten minutes the animal is placed under the laser and the ear is folded back to expose the ventral side. The laser is positioned in the center of the ear and focused to an intensity of 8.4-9.0 V (with is generally ˜4.5 cm above the ear). Data acquisition is initiated with a 15 by 15 image format, auto interval, 60 images and a 20 sec time delay with a medium resolution. Test compounds are administered following the 10th image via injection into the peritoneal space. Images 1-10 are considered the animal's baseline and data is normalized to an average of the baseline mean intensities.

Materials and Methods—Laser Doppler Pirimed PimII; Niacin (Sigma); Nembutal (Abbott Labs).

### Inhibition of Free Fatty Acid Production In Vivo, in Male Sprague-Dawley Rats

Non-esterified free-fatty acid (NEFA) assays are done on serum derived from live, freely moving rats. Catheters are surgically implanted into femoral veins and the animals are used within one week of arrival. Food is removed from the animals approximately 16 hours prior to the assay. A draw of ˜200 μl blood is pulled from the catheter and represents the baseline NEFA serum sample. Drug is administered intra-peritoneally (IP) or orally (po) at various concentrations to individual rats and then ˜200 μl blood draws are pulled from the catheter at the indicated time points for further NEFA analysis. NEFA assays are performed according to the manufacturer's specifications (Wako Chemicals, USA; NEFA C) and free fatty acid concentrations are determined via regression analysis of a known standard curve (range of known free fatty acids). Data is analyzed using Excel and PrismGraph.

All patents, patent applications and publications that are cited herein are hereby incorporated by reference in their entirety. While certain preferred embodiments have been described herein in detail, numerous alternative embodiments are seen as falling within the scope of the invention.

What is claimed is:

1. A compound represented by Formula I: 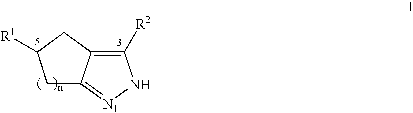
or a pharmaceutically acceptable salt or solvate thereof
wherein:
n represents 1 or 2;
R¹ is selected from the group consisting of cyclohexyl, phenyl and heteroaryl containing 5-6 atoms, said Heteroaryl 5-membered rings containing 1-4 heteroatoms, 0-1 of which are O or S and 0-4 of which are N, and said Heteroaryl 6-membered rings containing 1-3 N atoms,
said cyclohexyl, phenyl and heteroaryl being optionally substituted with 1-4 members selected from the group consisting of: halogen, OH, SH, CN, nitro, C₁₋₄ haloalkyl, amino, C₁₋₄ alkylamino, C₂₋₈ dialkylamino, C₁₋₄ alkyl, C₁₋₄ alkoxy, C₂₋₄alkenyl, C₂₋₄alkynyl, C₃₋₆ cycloalkyl, C₁₋₄haloalkoxy, C₁₋₄alkylthio, C₁₋₄alkylsulfinyl, and C₁₋₄alkylsulfonyl, and
R₂ is 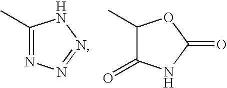
or CO₂R^{a} wherein R^{a} is H or C₁₋₄alkyl.

2. A compound in accordance with claim 1 wherein n is 1.

3. A compound in accordance with claim 1 wherein n is 2.

4. A compound in accordance with claim 1 wherein R¹ represents phenyl or heteroaryl, said group being optionally substituted with 1-4 groups, 1-4 of which are halo groups and 1-2 of which are selected from the group consisting of: OH, SH, CN, nitro, C₁₋₄ haloalkyl, amino, C₁₋₄ alkylamino, C₂₋₈ dialkylamino, C₁₋₄ alkyl, C₁₋₄ alkoxy, C₂₋₄alkenyl, C₂₋₄alkynyl, C₃₋₆ cycloalkyl, C₁₋₄haloalkoxy, C₁₋₄alkylthio, C₁₋₄alkylsulfinyl, and C₁₋₄alkylsulfonyl.

5. A compound in accordance with claim 4 wherein R¹ represents phenyl optionally substituted with 1-4 groups, 1-4 of which are halo groups and 1-2 of which are selected from the group consisting of: OH, SH, CN, nitro, C₁₋₄ haloalkyl, amino, C₁₋₄ alkylamino, C₂₋₈ dialkylamino, C₁₋₄ alkyl, C₁₋₄ alkoxy, C₂₋₄alkenyl, C₂₋₄alkynyl, C₃₋₆ cycloalkyl, C₁₋₄haloalkoxy, C₁₋₄alkylthio, C₁₋₄alkylsulfinyl, and C₁₋₄alkylsulfonyl.

6. A compound in accordance with claim 4 wherein R¹ represents heteroaryl optionally substituted with 1-4 groups, 1-4 of which are halo groups and 1-2 of which are selected from the group consisting of: OH, SH, CN, nitro, C₁₋₄ haloalkyl, amino, C₁₋₄ alkylamino, C₂₋₈ dialkylamino, C₁₋₄alkyl, C₁₋₄ alkoxy, C₂₋₄alkenyl, C₂₋₄alkynyl, C₃₋₆ cycloalkyl, C₁₋₄haloalkoxy, C₁₋₄alkylthio, C₁₋₄alkylsulfinyl, and C₁₋₄alkylsulfonyl.

7. A compound in accordance with claim 6 wherein R¹ represents heteroaryl optionally substituted with 1-4 groups, 1-4 of which are halo groups and 1-2 of which are C₁₋₄ haloalkyl or C₁₋₄ alkyl.

8. A compound in accordance with claim 5 wherein R¹ represents phenyl optionally substituted with 1-4 halo groups.

9. A compound in accordance with claim 1 wherein R² represents CO₂R^{a} and R^{a} represents H.

10. A compound in accordance with claim 1 wherein R² represents tetrazolyl.

11. A compound in accordance with claim 1 selected from one of the following tables:
TABLE A 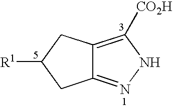
R¹ = 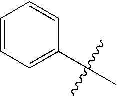 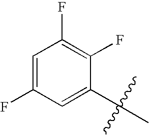 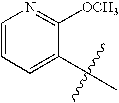 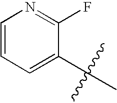 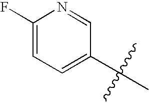 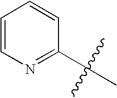 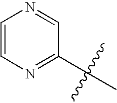 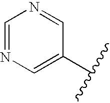 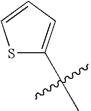 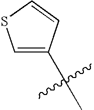 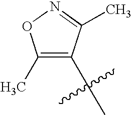 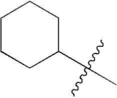 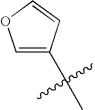 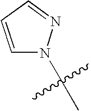 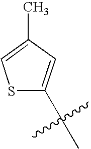 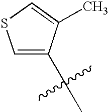 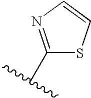 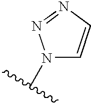 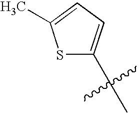 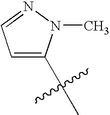 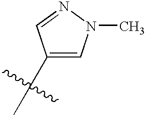 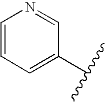 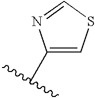 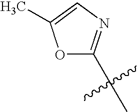
or a pharmaceutically acceptable salt or solvate thereof;
TABLE B 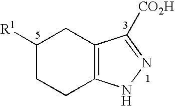
R¹ = 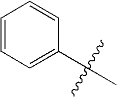 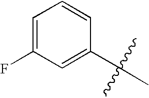 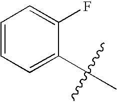 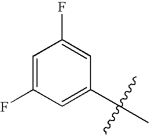 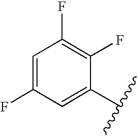 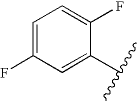 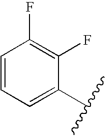 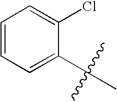 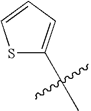 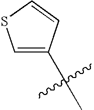 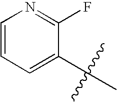 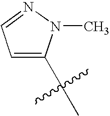 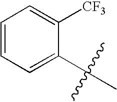 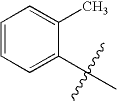
or a pharmaceutically acceptable salt or solvate thereof;
TABLE C 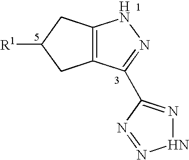
R¹ = 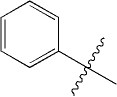 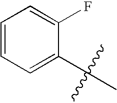 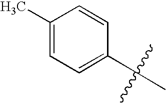 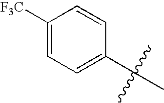 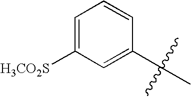 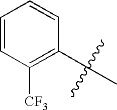 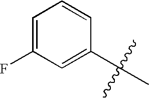 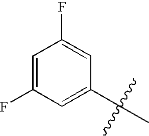 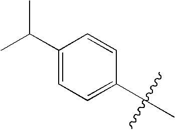 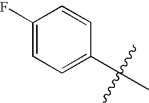 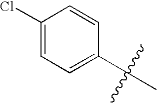 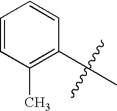 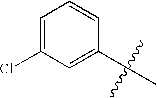 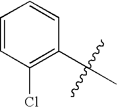 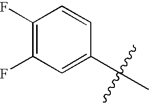 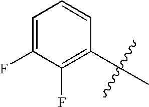 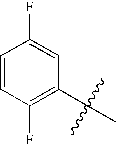 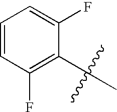 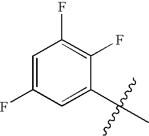 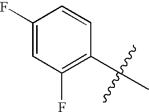 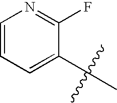 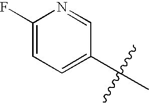 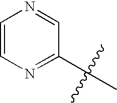 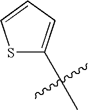 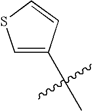 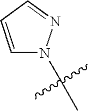 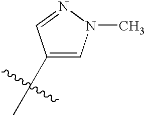
or a pharmaceutically acceptable salt or solvate thereof, and
TABLE D 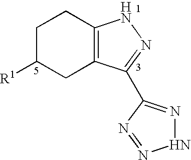
R¹ = 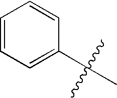 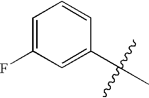 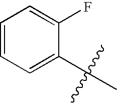 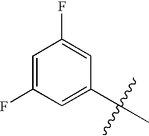 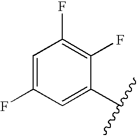 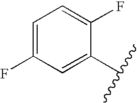 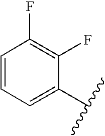 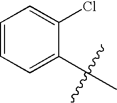 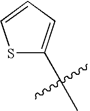 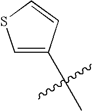 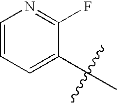 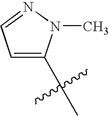 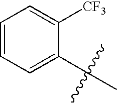 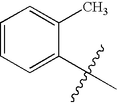
or a pharmaceutically acceptable salt or solvate thereof.

12. A pharmaceutical composition comprising a compound in accordance with claim 1 in combination with a pharmaceutically acceptable carrier.

13. A method of treating atherosclerosis in a human patient in need of such treatment comprising administering to the patient a compound of claim 1 in an amount that is effective for treating atherosclerosis.

14. A method of treating dyslipidemia in a human patient in need of such treatment comprising administering to the patient a compound of claim 1 in an amount that is effective for treating dyslipidemias.

15. A method of treating diabetes in a human patient in need of such treatment comprising administering to the patient a compound of claim 1 in an amount that is effective for treating diabetes.

16. A method of treating metabolic syndrome in a human patient in need of such treatment comprising administering to the patient a compound of claim 1 in an amount that is effective for treating metabolic syndrome.

17. A method of treating atherosclerosis, dyslipidemias, diabetes, metabolic syndrome or a related condition in a human patient in need of such treatment, comprising administering to the patient a compound of claim 1 and a DP receptor antagonist, said compounds being administered in an amount that is effective to treat atherosclerosis, dyslipidemia, diabetes, metabolic syndrome or a related condition in the absence of substantial flushing.

18. A method of treating atherosclerosis, dyslipidemias, diabetes or a related condition in a human patient in need of such treatment, comprising administering to the patient a compound of claim 1 and a DP receptor antagonist selected from the group consisting of compounds A through AJ:

| | |
|---|---|
| | Compound A |
| 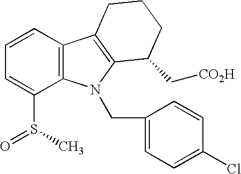 | |
| | Compound B |
| 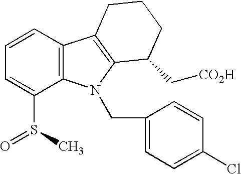 | |
| | Compound C |
| 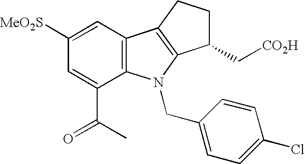 | |
| | Compound D |
| 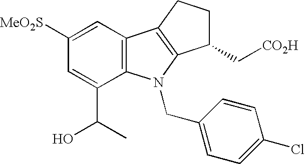 | |
| | Compound E |
| 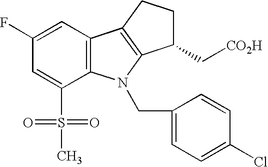 | |
| | Compound F |
| 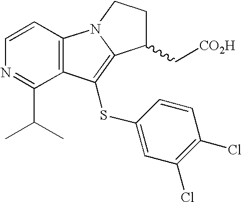 | |
| | Compound G |
| 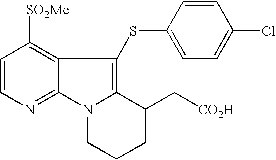 | |
| | Compound H |
| 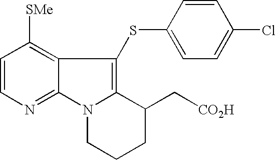 | |
| | Compound I |
| 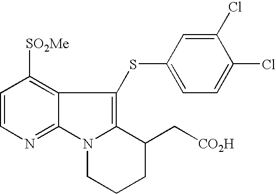 | |
| | Compound J |
| 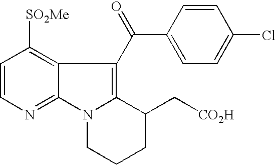 | |
| | Compound K |
| 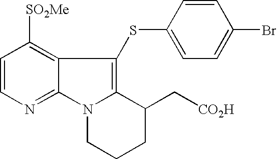 | |
| | Compound L |
| 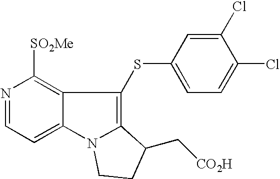 | |
| | Compound M |
| 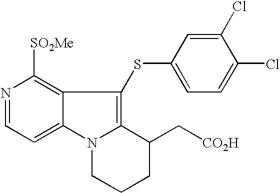 | |
| | Compound N |
| 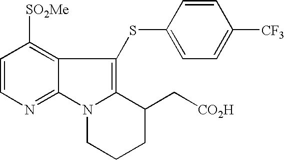 | |
| | Compound O |
| 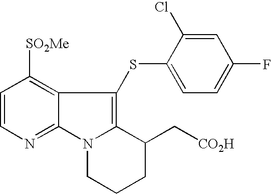 | |
| | Compound P |
| 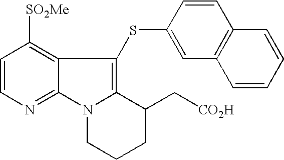 | |
| | Compound Q |
| 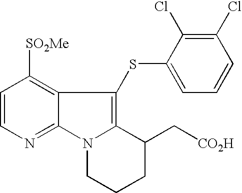 | |
| | Compound R |
| 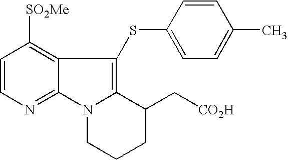 | |
| | Compound S |
| 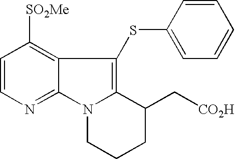 | |
| | Compound T |
| 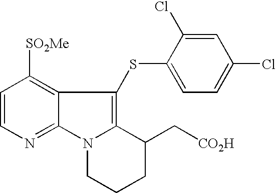 | |
| | Compound U |
| 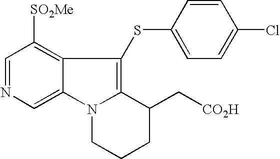 | |
| | Compound V |
| 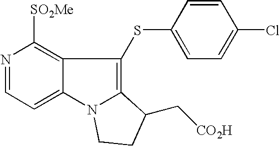 | |
| | Compound W |
| 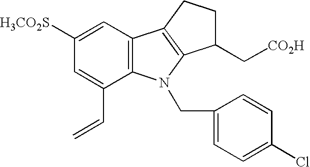 | |
| | Compound X |
| 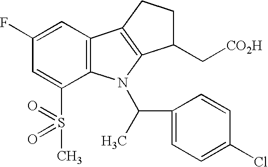 | |
| | Compound Y |
| 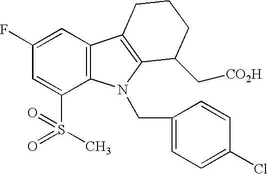 | |
| | Compound Z |
| 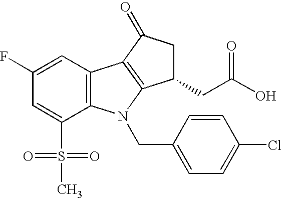 | |
| | Compound AA |
| 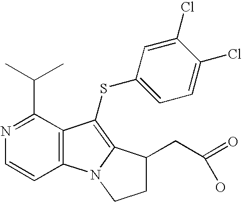 | |
| | Compound AB |
| 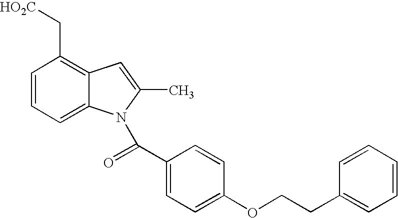 | |
| | Compound AC |
| 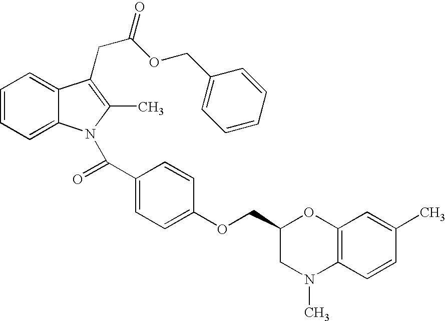 | |
| | Compound AD |
| 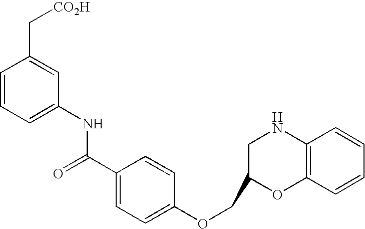 | |
| | Compound AE |
| 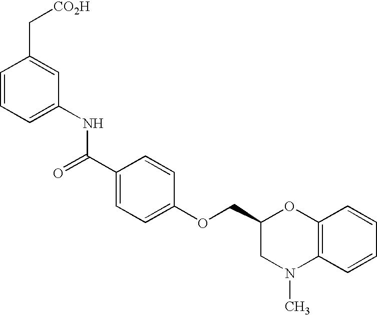 | |
| | Compound AF |
| 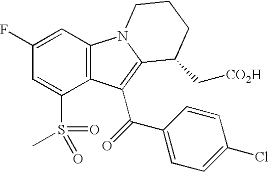 | |
| | Compound AG |
| 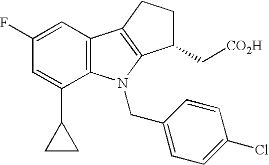 | |
| | Compound AH |
| 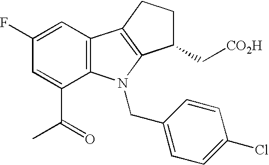 | |
| | Compound AI |
| 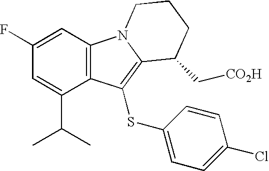 | |
| | Compound AJ |
| 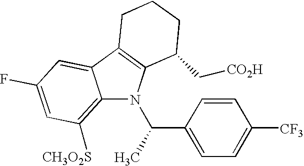 | |

or a pharmaceutically acceptable salt or solvate thereof.
